(19) United States
(12) Patent Application Publication
(10) Pub. No.: US 2016/0220170 A1
(43) Pub. Date: Aug. 4, 2016

(54) BIOLOGICAL INFORMATION MEASUREMENT SYSTEM

(71) Applicant: TOTO LTD., Kitakyushu-shi (JP)

(72) Inventors: Aya HASEGAWA, Kitakyushu-shi (JP); Tetsuhiro WASADA, Kitakyushu-shi (JP); Aya TAKAO, Kitakyushu-shi (JP); Satoko KIZUKA, Kitakyushu-shi (JP); Hidenori OKA, Kitakyushu-shi (JP); Akemi TAKESHITA, Kitakyushu-shi (JP); Masayuki NAGAISHI, Kitakyushu-shi (JP); Koji SONODA, Kitakyushu-shi (JP); Shingo YAMAYA, Kitakyushu-shi (JP); Hiroshi TSUBOI, Kitakyushu-shi (JP)

(21) Appl. No.: 15/010,974

(22) Filed: Jan. 29, 2016

(30) Foreign Application Priority Data
Jan. 30, 2015 (JP) 2015-017454
Nov. 27, 2015 (JP) 2015-232233

(51) Int. Cl. A61B 5/00; G06F 19/28; A61B 10/00

(52) U.S. Cl. CPC A61B 5/42; A61B 10/00; G06F 19/28; A61B 2010/0083

(57) ABSTRACT

There is provided a diagnosis system that is capable of allowing general consumers to readily purchase it, as well as capable of measuring defecation gas at home. The present invention is a system (1) including a device (10) on a test subject side, and a server (12), the test subject side device includes a suction device, a gas detector, a test subject identification device, a control device, and a communication device, the server has a database (12a) in which first detection data of odiferous gas, and test subject disease data are accumulated and recorded, and the server side data analyzer is provided with relating means that generates affected test subject defecation gas data, similarity determination means that compares a time-dependent change characteristic of the affected test subject defecation gas data, and a time-dependent change characteristic of test subject defecation gas data.

### RELATED APPLICATIONS

This application claims priority under 35 U.S.C. §119 to Japanese Patent Application Nos. 2015-017454 filed on Jan. 30, 2015 and 2015-232233 filed on Nov. 27, 2015, the entire contents of which are hereby incorporated by reference.

### BACKGROUND OF THE INVENTION

1. Field of the Invention

The present invention relates to a biological information measurement system, and more particularly to a biological information measurement system that measures physical condition of a test subject on the basis of defecation gas discharged in a bowl of a flush toilet.

2. Description of the Related Art

In recent years, a mortality rate caused by cancer extremely decreases due to evolution of a diagnosis technique for serious illness, such as cancer, and of a technique of cancer treatment, with evolution of medical technology. However, presenting to a hospital at regular intervals for diagnosis to prevent cancer burdens a patient. In contrast, many patients actually present to a hospital after realizing wrong physical condition, and thus unfortunately still many people have cancer. In addition, no practical device for preventing cancer has been developed yet, so that it cannot be said that cancer prevention is sufficiently achieved.

In light of the circumstances, the present inventors have studied for a long time with a strong desire for manufacturing a device that is really required in the market, such as a device capable of more simply and easily diagnosing serious illness, such as cancer, at home without presenting to a hospital, to achieve prevention or early treatment of serious illness.

The present applicants have developed devices, such as: a device that is mounted in a seat of a Western-style toilet to collect defecation gas discharged into a bowl when a test subject defecates to acquire the amount of stool discharged on the basis of a concentration of carbon dioxide contained in the defecation gas as a biological information index (refer to Patent Literature 1: Japanese Patent No. 5131646); and a device in which a deodorizing device assembled in a seat of a flush toilet sucks defecation gas that is discharged together when a test subject defecates so that a carbon dioxide gas sensor measures a concentration of carbon dioxide of the gas sucked to allow intestinal conditions of a test subject to be estimated on the basis of the measured concentration of carbon dioxide (refer to Patent Literature 2: Japanese Patent No. 5019267). Unfortunately, these devices estimate only current intestinal conditions, so that it is impossible to achieve a purpose of the present inventors to enable serious illness, such as cancer, to be simply and easily diagnosed, as well as to enable a risk state of the serious illness to be simply and easily acquired. In addition, there is also known a fart detector in which gas sensor is arranged so as to be brought into contact with air near an excretory organ of a human to detect a fart on the basis of a peak value of output of the gas sensor (refer to Patent Literature 3: Japanese Patent Laid-Open No. 2003-90812). In the fart detector, a tube inserted into an excretory organ of a patient staying in bed in a diaper or underwear worn by the patient is drawn, and air is sucked through the tube by a suction pump to collect a fart of the patient. In addition, the fart detector only distinguishes a fart and urination on the basis of a half-value width of a peak value of output of the gas sensor so that a doctor checks whether a fart is discharged after an appendix operation, or time to replace a diaper is detected, whereby it is impossible to achieve the purpose of the present inventors. Meanwhile, Japanese Patent Laid-Open No. 2014-160049 (Patent Literature 4) discloses a portable type apparatus for measuring a risk of colorectal cancer that includes a sensor for measuring methyl mercaptan gas from components of a fart discharged by a test subject, a calculation unit for calculating a concentration of the methyl mercaptan gas measured by the sensor, and a display, to estimate a risk of acquiring colorectal cancer.

Japanese Patent Laid-Open No. 9-43182 (Patent Literature 5) describes a biological monitoring device. In the biological monitoring device, a fabric T-bandage to which gas sensor is attached is provided so that the gas sensor is arranged near an anus to detect a fart discharged from the anus. A signal from the gas sensor is transmitted to a processor to be stored in a memory. It is also known that data stored in a memory is compared with previous data, and that a warning is displayed in a display device if there is abnormality, such as a large difference.

Japanese Patent No. 3525157 (Patent Literature 6) describes a method of measuring components of flatus. In the method of measuring components of flatus, a sampling tube is arranged at a portion in a seat of a toilet. When a person to be measured turns on a main switch of a device, a suction pump is operated to suck gas near an anus. An index gas detector always measures a concentration of carbonic acid gas in the gas sucked, and a control/arithmetic processing unit recognizes that a flatus has been diffused if the concentration measured steeply increases. If a flatus is diffused, another suction pump starts operating to allow a part of gas sucked to be inserted into a sample measuring tube. An inserted sample is fed into a column so that gas components are separated to be ionized. It is also known that the amount of ionization is converted into an electric signal so that a concentration of gas components of a detection object in the flatus is measured.

Japanese Patent Laid-Open No. 2014-206945 (Patent Literature 7) describes a health information utilization system. In the health information utilization system, personal health information on health management, inputted from a terminal device, is individually stored in a database of each of a plurality of data centers, and an analysis server device reads out the personal health information to analyze it. A big data creation server device searches the personal health information under a specific condition to create big data and store it. The health information utilization system allows health content based on knowledge in a special field to be browsed at a terminal device, and stores the personal health information in the plurality of data centers to manage it, as well as allows a health determination result acquired by applying automatic determination processing to the personal health information, and a health determination result acquired by determination processing applied by an expert, to be browsed at a terminal. The system described above is also known.

In order to develop a device capable of diagnosing serious illness, such as cancer, in recent years, it has been known that there is a correlation between disease of colorectal cancer and components of flatus contained in a fart and a stool. Specifically, colorectal cancer patients have more methyl mercaptan gas containing a sulfur component, in components of flatus, as compared with healthy people.

Components of flatus are discharged along with a stool, as a fart and defecation gas, during defecation. Thus, the present inventors, as published in Nihon Keizai Shimbun issued Jan. 5, 2015, have studied on the assumption that measuring a specific gas, such as methyl mercaptan gas, in a fart and defecation gas, discharged during defecation, enables colorectal cancer in the intestine to be found out, as with Patent Literature 4 above, and the like. However, a measuring device capable of accurately measuring only this specific gas, such as methyl mercaptan gas, is very expensive and large in size. In addition, methyl mercaptan gas is contained in minute amount in defecation gas, and is contained in less amount than the minute amount in a stage before getting cancer. As a result, it is very difficult to measure the methyl mercaptan gas, and thus the present inventors have been faced with a problem in which it is not realistic in cost and size that at least this kind of gas analyzer capable of accurate measurement is assembled in a household toilet device to be widely used as a consumer product.

Further, the object of the health information utilization system described in Patent Literature 7 is to create big data by analyzing a great deal of personal health information stored in a data center, and utilize the data in health care of each individual. However, the health information utilization system does not analyze a specific risk of a user suffering from a disease or notify the user of the specific risk, and cannot achieve the object of the present inventors.

The present inventors desire to reduce the number of people who have a serious illness, such as cancer, as far as possible. To achieve this object, the present inventors continue to study by having strong feeling for necessity of providing a device that is capable of allowing general consumers to readily purchase it, and capable of simply and easily performing diagnosis at home, and then finally find out a technical solution for realizing the device.

It is an object of the present invention to provide a diagnosis system that is capable of allowing general consumers to readily purchase it, as well as capable of measuring defecation gas at home to prevent people from having a serious disease, such as a cancer, or urging people to present to a hospital to receive treatment under a moderate condition, the diagnosis system being really required in the market, having high practicality.

### SUMMARY OF THE INVENTION

In order to solve the above described problem, the present invention is a biological information measurement system that measures physical condition of a test subject on the basis of defecation gas discharged into a bowl of a flush toilet, the biological information measurement system including: a test subject side device provided in a room where the flush toilet is installed; and a server communicable with the test subject side device, wherein the test subject side device includes a suction device that sucks gas in the bowl into which the defecation gas is discharged during a defecation act of the test subject, a gas detector that is sensitive to methyl mercaptan gas that is odiferous gas containing a sulfur component and odiferous gas other than methyl mercaptan gas, which are contained in the gas sucked by the suction device, and outputs first detection data, a test subject identification device that accepts input of test subject identification information, a control device that controls the suction device and the gas detector, and a communication device that transmits the first detection data of the odiferous gas detected by the gas detector to the server, the server has a database in which test subject defecation gas data including the first detection data of the odiferous gas transmitted from a plurality of the test subject side devices, the test subject identification information and dates and times of the defecation acts, and test subject disease data concerning diseases which affect a plurality of test subjects using the test subject side devices, which is acquired from a medical facility, are accumulated and recorded, and a server side data analyzer that analyzes the physical condition of a test subject on the basis of the test subject defecation gas data and the test subject disease data which are accumulated and recorded in the database, and the server side data analyzer includes relating means that generates affected test subject defecation gas data by relating the test subject disease data of a test subject affected by a predetermined disease, and the test subject defecation gas data of the test subject with each other, and accumulates the affected test subject defecation gas data in the database, similarity determination means that compares a time-dependent change characteristic of the affected test subject defecation gas data accumulated in the database, and a time-dependent change characteristic of the test subject defecation gas data of a specific test subject transmitted from the test subject side device, and determines whether or not the time-dependent change characteristic of the affected test subject defecation gas data and the time-dependent change characteristic of the test subject defecation gas data are similar to each other, and notification means that performs a predetermined notification to the test subject or a previously registered person who relates to said test subject, associated with the test subject defecation gas data which is determined as having similarity to the affected test subject defecation gas data by the similarity determination means.

Heretofore, there has been actually no effective device other than diagnosis at hospital for checking whether people have serious illness, such as cancer, or for checking people for prevention of serious illness. In contrast, according to the present invention, general consumers can simply and easily purchase the device to perform measurement at home. In addition, it is possible to allow a test subject to be prevented from having a serious disease, such as cancer, or to present to a hospital to receive treatment under a moderate condition, by only performing an excretory act as usual to measure defecation gas discharged during defecation without making an effort to perform additional measurement action. In this way, the present invention achieves an excellent effect of enabling a device that is really required in the market to be realized and a diagnosis system having high practicality to be provided.

Before advantageous effects of the present invention is specifically described, a technical idea of allowing a system to be widely used at standard home as a consumer product will be described. Key point of the idea are reverse thinking and effective simplified knowledge acquired by understanding characteristics of serious illness, such as cancer, and using the characteristics.

Specifically, one of key points of a system of the present invention is acquired by reverse thinking of a device installed at each home by which people are not diagnosed as having serious illness, such as cancer. That is, a test subject of general consumers who purchase the devices really wants to know whether the test subject is in a stage before having cancer (hereinafter this stage is referred to as ahead-disease), instead of whether the test subject has cancer, to recognize an increasing risk of cancer to improve a future life to preventing having cancer. Thus, it is thought that a device capable of allowing health people to accurately recognize a risk of cancer to improve physical condition for preventing having cancer is worth to a device required at standard home.

Another key point of the system of the present invention is acquired by a simplified idea that a device capable of diagnosing a specific kind of cancer, such as a rectal cancer, or diagnosing an increasing risk of a specific kind of cancer, is unnecessary. The idea is acquired from characteristics of a test subject who is anxious about any kind of cancer instead of about a specific kind of cancer, such as a rectal cancer. Thus, the inventors have simply thought that accuracy of measurement capable of identifying a kind of cancer is unnecessary, on the basis of an assumption that it is quite unnecessary to identify a kind of cancer instead of an assumption that device has a commercial value if diagnosing a specific kind of cancer.

Specific effects of a system in accordance with the present invention configured on the basis of the knowledge and the effective simplified idea described above will be described below.

In the present invention, since defecation gas discharged into a bowl of a toilet is measured to analyze physical condition of a test subject, it is possible to perform diagnosis by allowing a test subject to only defecate as usual without requiring an effort to perform measurement action. Requiring no effort allows the test subject to have no burden, so that it is possible to continue measurement for a long time to reliably acquire information on a change in health condition, and on a state where a risk of cancer is increasing.

In addition, in the present invention, no sensor for measuring methyl mercaptan gas at a pinpoint is used, and a sensor that is widely sensitive also to odiferous gas other than the methyl mercaptan gas, in defecation gas, is used. If the sensor for measuring methyl mercaptan gas at a pinpoint is used, it is possible to reliably detect a colorectal cancer because there is a correlation between the amount of methyl mercaptan gas and a colorectal cancer, and also to reliably find that a risk of cancer is increasing from the amount thereof. However, it is found that it is impossible to determine that a risk of cancer is increasing unless a risk of cancer increases to some extent to increase the amount of methyl mercaptan gas, whereby the sensor is unsuitable for the present invention having an object to prevent people from having cancer.

In contrast, the sensor that is widely sensitive to odiferous gas is capable of detecting not only a state where a risk of cancer is increasing, but also a risk of cancer from wrong physical condition. Specifically, first if a risk of cancer increases, a very strong odiferous gas containing a sulfur component, such as methyl mercaptan gas or hydrogen sulfide, increases in amount. Then, the sensor that is widely sensitive to odiferous gas is capable of detecting increase of this kind of gas. Thus, even if a sensor that is widely sensitive to odiferous gas other than methyl mercaptan gas in defecation gas is used, it is possible to determine that a risk of cancer increases. Accordingly, the sensor that is widely sensitive also to odiferous gas serves also as a sensor for measuring methyl mercaptan gas at a pinpoint in this point.

The present invention uses a gas detector that is sensitive not only to methyl mercaptan gas but also to odiferous gas other than methyl mercaptan gas, in defecation gas, so that only the amount of odiferous gas in the defecation gas can be detected, but the amount of methyl mercaptan gas cannot be measured, whereby it is impossible to accurately identify a state of cancer. However, the present inventors find out that using gas detector that is sensitive not only to methyl mercaptan gas, but also to odiferous gas other than methyl mercaptan gas, in defecation gas, allows a device to effectively serve as a device for preventing a state where a risk of cancer increases in healthy people, and a risk, such as having cancer. Specifically, healthy people have a small total amount of methyl mercaptan gas and odiferous gas other than the methyl mercaptan gas. In contrast, a total amount of methyl mercaptan gas and odiferous gas other than the methyl mercaptan gas temporarily increases due to deterioration of intestinal environment other than having cancer. The deterioration of intestinal environment is specifically caused by the following, such as excessive obstipation, a kind of meal, lack of sleep, crapulence, excessive drinking, and excessive stress. It can be said that each of these causes is a bad living habit. The bad living habit will result in cancer, however, there is no means of recognizing a risk of cancer state even if the risk of cancer increases, and thus many people continue the bad living habit on the basis of a convenient assumption that the many people themselves survive.

In this way, performing the bad living habit as described above increases all or any one of odiferous gases in defecation gas, such as methyl mercaptan, hydrogen sulfide, acetic acid, trimethylamine, or ammonia. In contrast, the present invention analyzes physical condition on the basis of detection data acquired by gas detector that detects not only methyl mercaptan gas, but also odiferous gases other than methyl mercaptan gas, such as hydrogen sulfide, acetic acid, trimethylamine, or ammonia, in defecation gas. Thus, an analysis result based on a total amount of the odiferous gas in the defecation gas reflects a result caused by a wrong physical condition and a bad living habit, of a test subject, so that the analysis result is usable as an index based on objective data for improving a physical condition and a living habit in which this kind of risk of cancer may increase, or is usable as an effective index for maintaining a health condition to reduce a risk of having cancer, whereby it is found that the analysis result acts on the object of improving a living habit and reducing a risk of cancer in an extremely effective manner to achieve an excellent effect.

In this way, the present invention measures methyl mercaptan gas and odiferous gas other than the methyl mercaptan gas to enable measurement capable of notifying a state where a risk of cancer may increase, and a suitable warning of having cancer if this kind of state continues for a long time, to a test subject. The so-called reverse thinking allows knowledge suitable for the object of reducing people having cancer to be found out.

In addition, since the present invention uses a sensor that is widely sensitive not only to methyl mercaptan gas but also to odiferous gas other than the methyl mercaptan gas, a device can be manufactured at low cost, thereby enabling the device to be provided as a consumer product. Accordingly, it is possible to sufficiently satisfy a request of test subjects that diagnosis can be simply and easily performed at home to prevent having a serious disease, such as cancer, or they can be urged to present to a hospital to receive treatment under a moderate condition.

According to the present invention configured as described above, the detection data of defecation gas is transmitted from the test subject side device to the server, by defecation performed every day, so that the test subject can perform health care without much effort. In addition, it becomes possible to notify a risk of a disease such as colorectal cancer at a stage of ahead-disease, by determination of similarity of the time-dependent change characteristic of the test subject defecation gas data transmitted from the test subject, and the time-dependent change characteristic of the affected test subject defecation gas data based on the information of a large number of test subjects accumulated in the database in the server. In the present invention, the risk is not evaluated by defecation gas data of one time, but similarity is evaluated on the basis of the time-dependent change characteristics, so that the precision of prediction of a risk can be made extremely high. It becomes possible to notify risks of affection of a large number of serious illnesses, and save test subjects at a stage of ahead-disease, by associating relationships between various gastrointestinal diseases and the time-dependent change characteristics of defecation gas data in the server side data analyzer.

In the present invention, it is preferable that the gas detector is configured to detect healthy-state gas composed of at least one of hydrogen gas, carbon dioxide gas, methane gas and acetic acid gas, contained in the defecation gas sucked by the suction device to output second detection data, the communication device is configured to transmit the second detection data of the healthy-state gas to the server with the first detection data, and the similarity determination means determines whether or not the affected test subject defecation gas data and the test subject defecation gas data are similar to each other on the basis of a time-dependent change characteristic of correlation of the odiferous gas and the healthy-state gas.

According to the present invention configured in this way, similarity is determined on the basis of the time-dependent change characteristic of the correlation of the odiferous gas and the healthy-state gas, so that an influence of noise or the like included at the time of measurement is hardly exerted, and an unnecessary mental burden can be prevented from being applied to a test subject by notifying the test subject of an erroneous analysis result.

In the present invention, it is preferable that for an analysis result of physical condition of a test subject by the server side data analyzer, a plurality of physical condition stages, from a healthy state to a state with concern for disease, are set, and the similarity determination means determines similarity at a time point at which a latest test subject defecation gas data of a specific test subject is deteriorated to a predetermined physical condition stage.

Even if a part of the test subject defecation gas data is similar to the affected test subject defecation gas data, in a state where physical condition of a test subject is not so bad, the physical condition of the test subject is often recovered thereafter, and the affection risk often disappears. If similarity determination is performed at a state in which the test subject is relatively healthy like this, and the affection risk is notified, an unnecessary mental burden is applied to the test subject. According to the present invention configured as described above, similarity is determined at the time point when the latest test subject defecation gas data of a test subject is deteriorated to a predetermined physical condition stage, so that proper notification can be given to the test subject when the risk is increased to such an extent that the affection risk should be notified.

In the present invention, it is preferable that a notification mode of notification by the notification means is varied in accordance with the physical condition stage based on the latest test subject defecation gas data of a test subject.

For example, in a state where the physical condition of a test subject is not so bad, information or the like useful for recovery of physical condition is necessary for the test subject, and in a state where an affection risk is increased to some degree, information or the like of medical facilities is useful for the test subject. According to the present invention configured as described above, the notification mode is changed in accordance with the physical condition stage of the latest test subject defecation gas data of the test subject, so that a proper person can be notified of proper information at a proper timing.

In the present invention, it is preferable that the notification means changes a person to be notified, in accordance with the physical condition stage based on the latest test subject defecation gas data of a test subject.

According to the present invention configured in this way, the person to be notified is changed in accordance with the physical condition stage of the latest test subject defecation gas data of the test subject, so that the test subject or a third party notified can take proper measures for recovery of health.

In the present invention, it is preferable that the notification means notifies an analysis result of physical condition in more detail, as the physical condition stage based on the latest test subject defecation gas data of a test subject is closer to the state with concern for a disease.

According to the present invention configured in this way, the analysis result of physical condition is notified in more detail, as the physical condition stage of the test subject is closer to the state with concern for a disease, so that the test subject can recognize the state of health of himself or herself in detail, when the test subject has physical condition requiring immediate medical treatment, and the test subject can be given a strong motivation for having consultation in a medical facility or the like.

In the present invention, it is preferable that the notification means notifies a disease which is feared to affect a test subject, and a risk of the test subject being affected after a predetermined period, when the physical condition stage based on the latest test subject defecation gas data of the test subject is on a side with more concern for the disease than a predetermined physical condition stage, and notifies whether or not a physical condition is improved when a latest physical condition stage is on a side of a healthy state.

According to the present invention configured in this way, proper information is notified in accordance with the physical condition stage of the test subject, so that the test subject performs proper health care, and can undergo diagnosis and medical treatment in accordance with necessity.

In the present invention, it is preferable that the relating means generates reference affected test subject defecation gas data to be a reference concerning a disease on the basis of the test subject defecation gas data of a plurality of test subjects affected by the same disease, and the similarity determination means compares time-dependent change characteristics of the reference affected test subject defecation gas data and the test subject defecation gas data, and determines whether or not the time-dependent change characteristics are similar to each other.

According to the present invention configured in this way, the reference affected test subject defecation gas data to be the reference concerning a disease is generated on the basis of the test subject defecation gas data of a plurality of test subjects affected by the same disease, so that an accurate reference can be set for a predetermined disease, and an affection risk can be estimated properly on the basis of similarity to the test subject defecation gas data.

In the present invention, it is preferable that the relating means is configured to classify test subjects using the test subject side devices into a plurality of groups, and generate the reference affected test subject defecation gas data for each of the groups, and the similarity determination means compares the test subject defecation gas data with the reference affected test subject defecation gas data generated in the group to which the test subject belongs, and determines whether or not the test subject defecation gas data is similar to the reference affected test subject defecation gas data.

According to the present invention configured in this way, the test subjects are classified into a plurality of groups, and the reference affected test subject defecation gas data is generated for each of the groups, so that precision of the reference affected test subject defecation gas data can be further increased. The test subject defecation gas data is compared with the reference affected test subject defecation gas data generated in the group to which the test subject belongs, so that precision of evaluation of an affection risk can be more increased.

In the present invention, it is preferable that the groups are classified on the basis of at least one of age, sex, district, occupation and a living environment.

According to the present invention configured in this way, the affection risk of the test subject is evaluated on the basis of the data of test subjects having similar physical constitutions to that of himself or herself, and precision of evaluation of the affection risk can be further increased.

In the present invention, it is preferable that the relating means is configured to evaluate accuracy of the affected test subject defecation gas data generated, and the notification means notifies the accuracy in addition.

According to the present invention configured in this way, the accuracy of the affected test subject defecation gas data is notified in addition, so that accuracy of the notified affection risk can be recognized more objectively, and an unnecessary mental burden can be prevented from being applied to a test subject by a reference with low accuracy.

In the present invention, it is preferable that the notification means changes a timing for performing notification, or a content of notification, in accordance with accuracy of the affected test subject defecation gas data to which the similarity is determined by the similarity determination means.

According to the present invention configured in this way, the timing for performing notification, or the content of notification is changed in accordance with accuracy of the affected test subject defecation gas data, so that an unnecessary mental burden can be prevented from being applied to a test subject by the test subject being notified of an affection risk on the basis of the affected test subject defecation gas data with low accuracy at a stage in which the affection risk is low.

In the present invention, the server side data analyzer is configured to allow the notification means to notify at least two kinds of information of health care information visualizing a change in the test subject defecation gas data in a time-dependent manner, and information based on determination of similarity to the affected test subject defecation gas data, so that a change in a state of health of the test subject can be recognized.

According to the present invention configured in this way, by notifying a relatively healthy test subject of the health care information, the test subject can recognize the change in the state of health of himself or herself in a time-dependent manner. Meanwhile, a test subject with an increasing affection risk is notified of similarity of the test subject defecation gas data and the affected test subject defecation gas data. Accordingly, the healthy test subject can be allowed to recognize the change in the stage of health without being given an unnecessary mental burden, and the test subject with an increasing affection risk can be notified of the risk properly.

In the present invention, it is preferable that the health care information is displayed so that the change in the state of health of the test subject can be recognized in a time-dependent manner, as a point in a physical condition display table provided with a first index based on the first detection data, and a second index based on the second detection data, and the server is configured to update the physical condition display table on the basis of inputted information on the test subject.

According to the present invention configured in this way, as the health care information, the state of health of the test subject is displayed in the physical condition table equipped with the first index and the second index, so that the test subject can understand the state of health of himself or herself from many aspects. The server updates the physical condition display table on the basis of the information on the test subject inputted, such as the test subject defecation gas data transmitted from each of test subjects, the test subject disease data, a result of the test subject having consultation in a medical facility or the like. Accordingly, the physical condition of the test subject is always displayed in the new physical condition table, so that the test subject can properly recognize the state of health of himself or herself.

In the present invention, it is preferable that the notification means is configured to perform notification so that a test subject can determine a timing at which a risk of the test subject being affected becomes high, with similarity of the test subject defecation gas data of the test subject and the affected test subject defecation gas data, which is determined by the similarity determination means.

According to the present invention configured in this way, notification is performed so that the test subject can determine the timing at which the risk of being affected becomes high, so that the test subject can recognize the affection risk of himself or herself more specifically, and the test subject can be given a clear motivation for having consultation in a medical facility and undergoing medical treatment.

In the present invention, it is preferable that the server side data analyzer further allows risk reduction information that is an attention for reducing a risk of a test subject being affected to be presented by the notification means.

According to the present invention configured in this way, the risk reduction information which is the measure for reducing the risk of the test subject being affected is notified, so that the test subject can immediately recognize the measure for recovering the physical condition of himself or herself, and can make an effort to recover the physical condition at an early stage.

In the present invention, it is preferable that in the database, test subject defecation gas data of respective test subjects, and the risk reduction information executed by the test subjects are recorded by being related with each other and accumulated, and the server side data analyzer allows the risk reduction information having a large risk reduction effect to be provided preferentially by the notification means.

According to the present invention configured in this way, the risk reduction information having a large risk reduction effect is preferentially notified, so that a test subject can easily recognize the measure with a high effect to recover physical condition, and can make use of the measure for recovery of the physical condition.

In the present invention, it is preferable that the gas detector is configured to also detect odiferous gas attached to a test subject which is detected before the test subject starts a defecation act, the communication device transmits detection data concerning the odiferous gas attached to the test subject to the server with the first detection data, and the server side data analyzer also uses the detection data concerning the odiferous gas attached to the test subject in analysis of physical condition of the test subject.

For example, it is known that in a liver disease, ammonia emitted as a body odor of a test subject increases. According to the present invention configured as described above, the odiferous gas attached to a test subject detected before a defecation act is started is also detected, and the data on the odiferous gas is also used in the analysis of the physical condition of the test subject, so that it becomes possible to use the biological information measurement system of the present invention in evaluation of affection risks of a larger number of diseases.

In the present invention, it is preferable that the test subject side device further includes diarrhea detection means capable of detecting diarrhea of a test subject, the communication device is configured to transmit information on diarrhea of the test subject to the server, the server side data analyzer analyzes a situation of prevalence of a disease on the basis of information on diarrhea collected from the respective test subject side devices, and when it is determined that the disease is prevalent, the server side data analyzer allows the notification means to notify that the disease is prevalent.

According to the present invention configured in this way, the server side data analyzer analyzes the situation of prevalence of the disease on the basis of the information on diarrhea collected from the respective test subject side devices, and when it is determined that the disease is prevalent, the server side data analyzer allows the notification means to notify that the diseases is prevalent, so that it becomes possible to notify facilities concerned of the occurrence of mass food poisoning or the like at an early stage to cope with the mass food poisoning quickly.

In the present invention, it is preferable that the server side data analyzer notifies a specific person or a facility as previously registered of information on a specific test subject as previously registered, or test subjects living in a specific district, which is acquired by the test subject side device, and is transmitted from the communication devices.

According to the present invention configured in this way, a specific person or a facility set in advance is notified of the information on a specific test subject set in advance, or a test subject living in a specific district, so that it becomes possible to monitor a state of health of a test subject who is suspected to be infected with an infectious disease or the like, and it becomes possible to take a measure to stop spread of the disease at an early stage.

The present invention is a server for biological information measurement that measures physical condition of a test subject on the basis of defecation gas discharged into a bowl of a flush toilet, having: a receiver that receives first detection data concerning methyl mercaptan gas which is odiferous gas containing a sulfur component in the defecation gas and odiferous gas other than methyl mercaptan gas, measured in a test subject side device; a database in which test subject defecation gas data including the first detection data of the odiferous gas, transmitted from a plurality of test subject side devices, test subject identification information for identifying test subjects, and dates and times when the first detection data are acquired, and test subject disease data concerning diseases which affect a plurality of test subjects who use the test subject side devices, acquired from a medical facility are accumulated and recorded; and a server side data analyzer that analyzes physical condition of a test subject on the basis of the test subject defecation gas data and the test subject disease data accumulated and recorded in the database, wherein the server side data analyzer includes relating means that generates affected test subject defecation gas data by relating the test subject disease data of a test subject affected by a predetermined disease, and the test subject defecation gas data of the test subject with each other, and accumulates the affected test subject defecation gas data in the database, similarity determination means that compares a time-dependent change characteristic of the affected test subject defecation gas data accumulated in the database, and a time-dependent change characteristic of the test subject defecation gas data of a specific test subject transmitted from the test subject side device, and determines whether or not the time-dependent change characteristic of the affected test subject defecation gas data and the time-dependent change characteristic of the test subject defecation gas data of the specific test subject are similar to each other, and notification means that performs a predetermined notification to the test subject or a previously registered person who relates to said test subject, associated with the test subject defecation gas data which is determined as having similarity to the affected test subject defecation gas data by the similarity determination means.

According to the biological information measurement system of the present invention, the highly practical biological information measurement system can be provided, which general consumers can readily purchase, prevents a user from being affected by a serious disease such as cancer by measurement of defecation gas at home, or can encourage a user in a less serious state to present to a hospital to undergo medical treatment, and is truly required by the market.

### BRIEF DESCRIPTION OF THE DRAWINGS

FIG. 1 shows a state in which a biological information measurement system in accordance with a first embodiment of the present invention is attached to a flush toilet installed in a toilet installation room;

FIG. 2 is a block diagram showing a configuration of the biological information measurement system of the first embodiment of the present invention;

FIG. 3 shows a configuration of a gas detector provided in the biological information measurement system of the first embodiment of the present invention;

FIG. 4 shows a flow of information sent to a server from test subject side devices, a hospital, companies and the like, in the biological information measurement system of the first embodiment of the present invention;

FIG. 5 shows a flow of information sent from the server to the test subject side devices, the hospital, the companies and the like, in the biological information measurement system of the first embodiment of the present invention;

FIG. 6 describes a flow of measurement of physical condition by the biological information measurement system of the first embodiment of the present invention;

FIG. 7 shows an example of a screen displayed in a display device of a remote control provided in the biological information measurement system of the first embodiment of the present invention;

FIG. 8 shows an example of a table of displaying physical condition displayed in the display device of the remote control provided in the biological information measurement system of the first embodiment of the present invention;

FIG. 9A shows an example of displacement of a plotted point of updated data by correction;

FIG. 9B shows limit processing with respect to the amount of displacement of a plotted point;

FIG. 10 shows an example of a diagnosis table displayed on a server of the biological information measurement system of the first embodiment of the present invention;

FIG. 11 is a graph schematically showing a detection signal of each of sensors provided in a biological information measurement system in one defecation act of a test subject;

FIG. 12A is a graph showing estimation of the amount of discharge of odiferous gas in a case where a reference value of residual gas is not fixed;

FIG. 12B is a graph showing an example of detection values acquired by a semiconductor gas sensor for measuring odiferous gas in a case where a test subject uses an alcoholic toilet seat disinfectant;

FIG. 13 shows an example of update of the diagnosis table;

FIG. 14 shows a configuration of a database provided in the server, in the biological information measurement system of the first embodiment of the present invention;

FIG. 15 is a flowchart showing a procedure of construction of the database, in the biological information measurement system of the first embodiment of the present invention;

FIG. 16 shows an example of detection data of defecation gas associated with information on a disease, in the biological information measurement system of the first embodiment of the present invention;

FIGS. 17A and 17B show examples of a reference affected test subject defecation gas data obtained by totalizing detection data of defecation gas associated with information on a disease, in the biological information measurement system of the first embodiment of the present invention;

FIG. 18 is a flowchart showing a procedure of similarity determination and notification by similarity determination means built in a server side data analyzer, in the biological information measurement system of the first embodiment of the present invention;

FIG. 19 shows an example of reference affected test subject defecation gas data and test subject defecation gas data having similarity, in the biological information measurement system of the first embodiment of the present invention;

FIG. 20 shows a physical condition state of a test subject in a diagnosis table, in the biological information measurement system of the first embodiment of the present invention;

FIG. 21 shows a mode of notification that is performed in accordance with the physical condition state of a test subject, in the biological information measurement system of the first embodiment of the present invention;

FIG. 22 shows an example of a risk display screen displayed in a display device of the test subject side device, in the biological information measurement system of the first embodiment of the present invention;

FIG. 23 shows an example of a disease determination screen displayed in a terminal of a medical facility, in the biological information measurement system of the first embodiment of the present invention;

FIG. 24 is a graph showing time-dependent change of gas attached to a test subject, such as ammonia, in the biological information measurement system of the first embodiment of the present invention;

FIG. 25 is a flowchart for detection of a disease which suddenly prevails, in the biological information measurement system of the first embodiment of the present invention;

FIG. 26 shows an example of a notification screen in a case where prevalence of a disease is detected, in the biological information measurement system of the first embodiment of the present invention;

FIG. 27 is a flowchart for prevention of epidemic of an infectious disease, in the biological information measurement system of the first embodiment of the present invention;

FIG. 28A shows a state in which a test subject side device of a biological information measurement system in accordance with another embodiment is attached to a flush toilet installed in a toilet installation room;

FIG. 28B is a perspective view showing a measuring device of the test subject side device shown in FIG. 28A;

FIG. 29 shows a configuration of a suction device of another embodiment of the present invention;

FIG. 30 shows a configuration of a gas detector in accordance with another embodiment of the present invention, the gas detector being configured to vary a reaching time of each of hydrogen gas and odiferous gas to the odiferous gas sensor to separate influence of the hydrogen gas;

FIG. 31 shows a detection waveform acquired by a semiconductor gas sensor of a gas detector, shown in FIG. 30;

FIG. 32 shows a result of measurement of the amount of healthy-state gas and odiferous gas contained in defecation gas acquired from each of healthy people less than sixties, healthy people in sixties to seventies, patients having early cancer, and patients having advanced cancer;

FIGS. 33A and 33B show the amount of hydrogen sulfide gas contained in defecation gas, compared between healthy people and patients having colorectal cancer;

FIGS. 34A and 34B show the amount of methyl mercaptan gas contained in defecation gas, compared between healthy people and patients having colorectal cancer;

FIGS. 35A and 35B show the amount of hydrogen gas contained in defecation gas, compared between healthy people and patients having colorectal cancer;

FIGS. 36A and 36B show the amount of carbon dioxide gas contained in defecation gas, compared between healthy people and patients having colorectal cancer;

FIGS. 37A and 37B show the amount of propionic acid gas contained in defecation gas, compared between healthy people and patients having colorectal cancer;

FIGS. 38A and 38B show the amount of acetic acid gas contained in defecation gas, compared between healthy people and patients having colorectal cancer; and

FIGS. 39A and 39B show the amount of butyric acid gas contained in defecation gas, compared between healthy people and patients having colorectal cancer.

### DETAILED DESCRIPTION OF THE PREFERRED EMBODIMENTS

One embodiment of a biological information measurement system of the present invention will be described in detail below with reference to drawings.

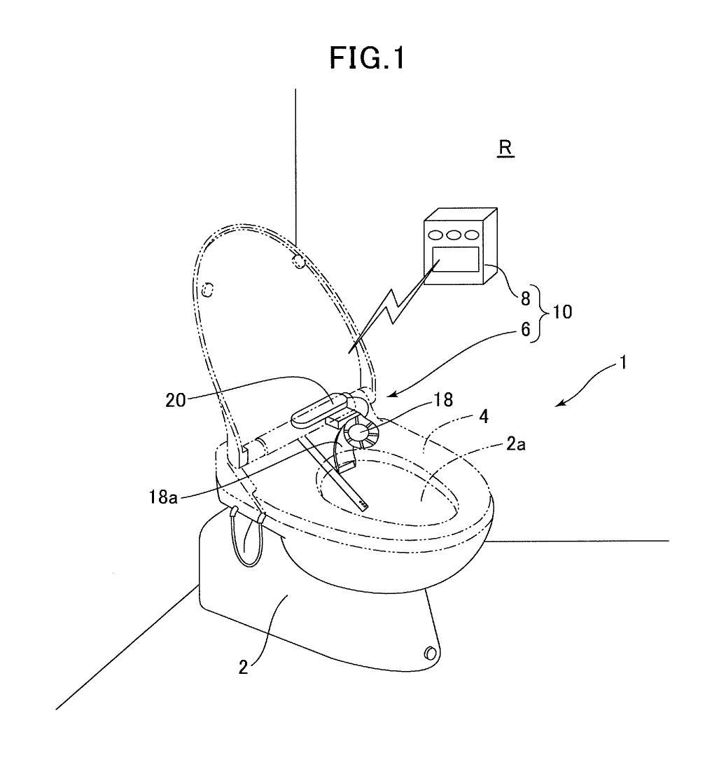

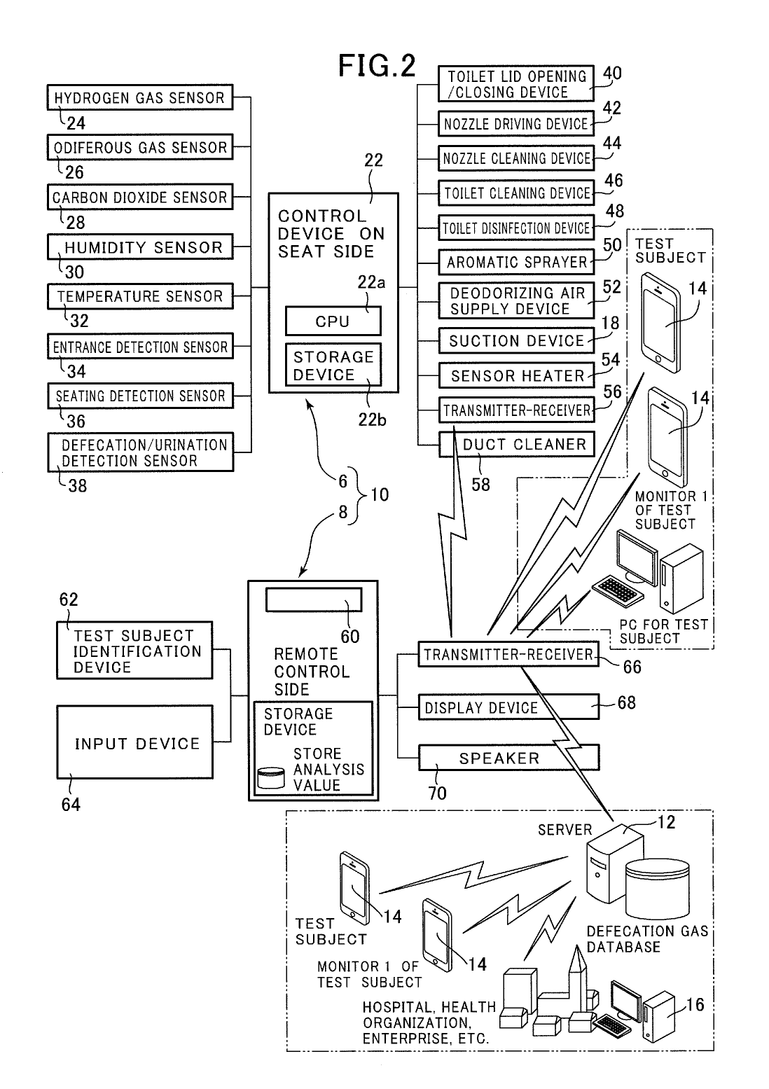

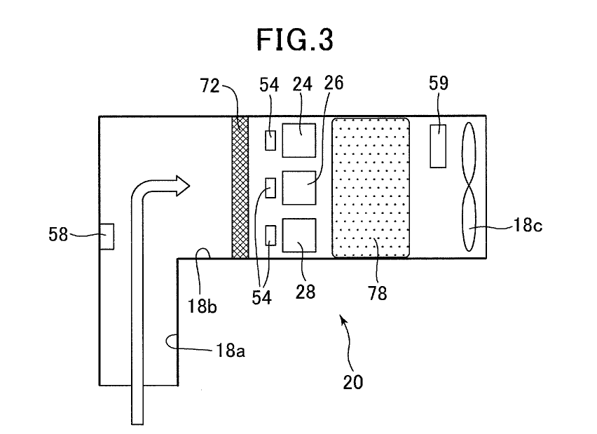

FIG. 1 shows a state in which a biological information measurement system in accordance with a first embodiment of the present invention is attached to a flush toilet installed in a toilet installation room. FIG. 2 is a block diagram showing a configuration of the biological information measurement system of the present embodiment. FIG. 3 shows a configuration of gas detector provided in the biological information measurement system of the present embodiment.

As shown in FIG. 1, the biological information measurement system 1 includes a measuring device 6 assembled inside a seat 4 mounted on a flush toilet 2 installed in a toilet installation room R, and a device 10 on a test subject side composed of a remote control 8 attached to a wall surface of the toilet installation room R. In addition, as shown in FIG. 2, the biological information measurement system 1 includes a server 12, a terminal 14 for a test subject, formed by installing dedicated software in a smartphone, and the like, and a medical facility terminal 16 installed in medical facilities, such as a hospital, to exchange data with the device 10 on a test subject side to serve as a part of the biological information measurement system 1. Further, measurement data transmitted from a large number of devices 10 on a test subject side is accumulated in the server 12 and the medical facility terminal 16, and then data analysis is performed.

The biological information measurement system 1 of the present embodiment analyzes physical condition including determination of cancer on the basis of odiferous gas containing a sulfur component, particularly a methyl mercaptan (CH₃SH) gas, in defecation gas discharged from a test subject during defecation. In addition, the biological information measurement system 1 of the present embodiment measures also healthy-state gas along with odiferous gas to improve analysis accuracy of physical condition on the basis of a correlation between the gases. The healthy-state gas originates from intestinal fermentation, and increases as an intestinal health degree increases. The healthy-state gas is specifically carbon dioxide, hydrogen, methane, short-chain fatty acid, and the like. In the present embodiment, a carbon dioxide gas and hydrogen gas, which are easy to be measured and are large in amount to enable reliability of measurement of a health index to be maintained at a high level, are measured as healthy-state gas. Each of the devices 10 on a test subject side is configured to display an analysis result during defecation of a test subject or immediately after the defecation. In contrast, the server 12 collects measurement results of a large number of test subjects to enable more detailed analysis by comparison with another test subject, and the like. In this way, in the biological information measurement system 1 of the present embodiment, the device 10 on a test subject side installed in the toilet installation room R performs a simple analysis, and the server 12 preforms a more detailed analysis.

Here, a measurement principle of physical condition in the biological information measurement system 1 of the present embodiment will be described.

Documents and the like report that if people have cancer of digestive system, particularly colorectal cancer, odiferous gas containing a sulfur component, such as methyl mercaptan or hydrogen sulfide, are discharged from an affected portion simultaneously with defecation. The digestive system includes the esophagus, stomach, duodenum, small intestine, large intestine, liver, the pancreas, and gallbladder. Although the large intestine also can be classified into the appendix, caecum, rectal, and colon, hereinafter the four portions are collectively called the large intestine. Cancer changes little on a daily basis, and gradually develops. If the cancer develops, the amount of odiferous gas containing a sulfur component, particularly methyl mercaptan, increases. That is, if the amount of odiferous gas containing a sulfur component increases, it can be determined that the cancer develops. In recent years, a concept of “ahead-disease” has spread, so that there is spread a concept of preventing a disease by improving physical condition at the time when the physical condition is deteriorated before falling sick. Thus, it is required to detect cancer, particularly progressive cancer, such as colorectal cancer, before having cancer, to improve physical condition.

Here, defecation gas discharged during defecation includes nitrogen, oxygen, argon, water vapor, carbon dioxide, hydrogen, methane, acetic acid, trimethylamine, ammonia, propionic acid, methyl disulfide, methyl trisulfide, and the like, along with hydrogen sulfide and methyl mercaptan. Among them, it is required to measure odiferous gas containing a sulfur-based component, particularly methyl mercaptan to determine disease of cancer. Each of the propionic acid, methyl disulfide, and methyl trisulfide, contained in defecation gas, is a very trace amount as compared with the methyl mercaptan, so that each of them does not matter to analysis of physical condition, such as determination of cancer, whereby it is possible to ignore them. However, it cannot be said that each of other gas components is a negligible trace amount. In order to accurately determine cancer, it is generally thought to use a sensor capable of detecting only odiferous gas containing a sulfur component. Unfortunately, the sensor for detecting only odiferous gas containing a sulfur component is large in size and very expensive, so that it is difficult to be configured as an apparatus for household use.

In contrast, the present inventors have diligently studied to reach an idea that a gas sensor that detects not only methyl mercaptan in defecation gas, but also odiferous gas including another odiferous gas, is used to enable an apparatus for household use to be configured at low cost. Specifically, the present inventors determine to use a general semiconductor gas sensor or a solid electrolyte sensor, sensitive not only to a sulfur-containing gas containing a sulfur component, but also to another odiferous gas, as a sensor for detecting gas.

If a risk of cancer increases, a very strong odiferous gas containing a sulfur component, such as methyl mercaptan gas, increases in amount. Then, a sensor, such as a semiconductor gas sensor, and a solid electrolyte sensor, widely sensitive to odiferous gas, is capable of always detecting increase of this kind of gas. Unfortunately, as described later, a sensor, such as a semiconductor gas sensor, and a solid electrolyte sensor, widely sensitive to an odiferous gas, detects also another odiferous gas, such as hydrogen sulfide, methyl mercaptan, acetic acid, trimethylamine, or ammonia, which increases when people have poor physical condition caused by a bad living habit. However, cancer is a disease developing for a long time, or a few years, so that a state of having an increased very strong odiferous gas containing a sulfur component, such as methyl mercaptan gas or hydrogen sulfide, continues for a long time if people have cancer. Thus, even if a general semiconductor gas sensor, or a solid electrolyte sensor, widely sensitive not only to sulfur-containing gas containing a sulfur component, but also to another odiferous gas, is used, it is possible to determine that there is a high possibility of disease of cancer to cause a risk of cancer to increase if the amount of gas is high for a long time.

In addition, a semiconductor sensor and a solid electrolyte sensor, using an oxidation-reduction reaction, detect not only methyl mercaptan gas, but also odiferous gas, such as acetic acid, trimethylamine, or ammonia, in defecation gas. However, the present inventors have discovered from experimental results that a mixed amount of odiferous gas, such as hydrogen sulfide, methyl mercaptan, acetic acid, trimethylamine, or ammonia, tends to increase if a bad living habit causes physical condition to be deteriorated, and tends to decrease if physical condition is good. Specifically, healthy people have a small total amount of methyl mercaptan gas and odiferous gas other than the methyl mercaptan gas. In contrast, a total amount of methyl mercaptan gas and odiferous gas other than the methyl mercaptan gas temporarily increases due to deterioration of intestinal environment caused by excessive obstipation, a kind of meal, lack of sleep, crapulence, excessive drinking, excessive stress, and the like.

Acetic acid in defecation gas tends to increase not only when physical condition is deteriorated due to diarrhea, and the like, but also when physical condition is good. That is, this tendency does not always agree with tendency of the amount of methyl mercaptan and another odiferous gas with change in physical condition described above. However, the amount of acetic acid contained in defecation gas is very small as compared with methyl mercaptan. Thus, even if the amount of acetic acid increases when physical condition is good, the amount of the increase is very small as compared with decrease in the amount of another odiferous gas. In addition, the amount of increase of acetic acid when physical condition is deteriorated due to diarrhea, and the like, is very large as compared with the amount of increase thereof when physical condition is good. Accordingly, the amount of odiferous gas contained in defecation gas tends to increase as a whole if physical condition is deteriorated due to a bad living habit, and tends to decrease if physical condition is good. Then, deterioration of intestinal environment due to this kind of bad living habit results in having cancer, so that the amount of odiferous gas contained in defecation gas is a suitable index to improve physical condition when people are still in a state before having cancer.

In the present embodiment, physical condition is analyzed on the basis of detection data acquired by a semiconductor sensor, or solid electrolyte sensor, sensitive not only to methyl mercaptan gas, but also to odiferous gas other than the methyl mercaptan gas, such as hydrogen sulfide, acetic acid, trimethylamine, ammonia, in defecation gas. Accordingly, it is possible to acquire an analysis result to which a result of a wrong physical condition and a bad living habit is reflected, and the analysis result is available as an index based on objective data for improving physical condition and a living habit that may increase a risk of cancer.

In addition, defecation gas contains not only odiferous gas, but also H₂ and methane, so that if a semiconductor gas sensor, or a solid electrolyte sensor, is used for a gas sensor, the gas sensor also reacts to H₂ and methane. Further, if a measuring device using a semiconductor gas sensor, or a solid electrolyte sensor, is set at each home, the sensor may react to an aromatic and a perfume.

In contrast, the present inventors, as described later in detail, achieve a method of removing influence of hydrogen and methane from detection data of a semiconductor gas sensor, or a solid electrolyte sensor, by using a hydrogen sensor, a methane sensor, and a column, and a method of removing influence of an aromatic and a perfume as noise by detecting defecation act. Accordingly, influence of hydrogen and methane, as well as influence of an aromatic and a perfume, is removed from data detected by the semiconductor gas sensor, or the solid electrolyte sensor, to enable the amount of only odiferous gas in defecation gas to be estimated.

The amount of methyl mercaptan and another odiferous gas contained in defecation gas is very small as compared with H₂ and methane. Accordingly, even if a semiconductor gas sensor, or a solid electrolyte sensor, is used, the amount of the mixed odiferous gas may not be accurately measured.

In contrast, the present inventors have paid attention to that healthy people have acidic intestinal environment, and that cancer patients have intestinal environment in which odiferous gas containing a sulfur component occurs to increase in amount, so that the intestinal environment becomes alkaline to reduce bifidobacteria, and the like, in amount, whereby the amount of healthy-state gas of ferment-base components, such as CO₂, H₂, or fatty acid, reliably and continuously decreases inversely with increase of the amount of odiferous gas.

Accordingly, the inventors have thought that even if measurement accuracy at each measurement is not always high, monitoring a correlation between the amount of odiferous gas, such as methyl mercaptan and the amount of healthy-state gas components, such as CO₂, or H₂ during defecation every day may enable occurrence of advanced cancer to be detected.

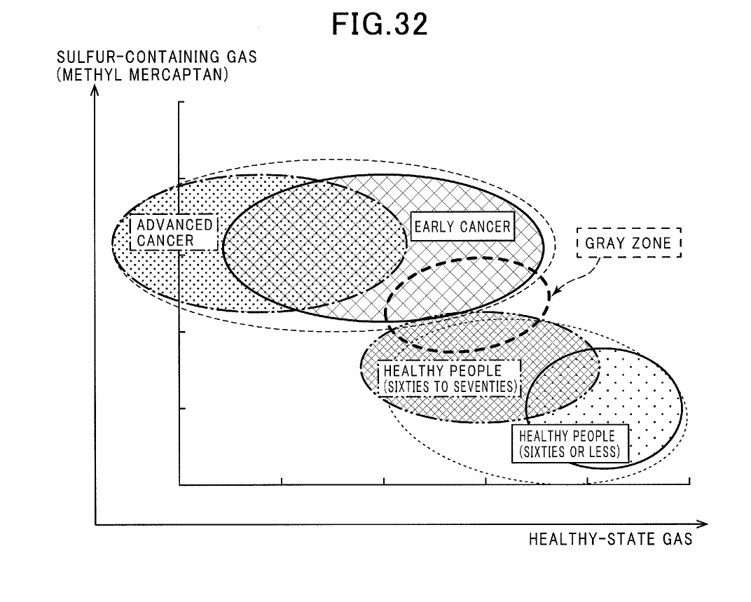

Then, the present inventors have measured the amount of healthy-state gas and odiferous gas contained in defecation gas acquired from each of healthy people less than sixties, healthy people in sixties to seventies, patients having early cancer, and patients having advanced cancer, and then a result shown in FIG. 32 has been acquired. That is, healthy people have defecation gas in which the amount of healthy-state gas is large, and the amount of odiferous gas is small. In contrast, cancer patients have defecation gas in which the amount of healthy-state gas is small, and the amount of odiferous gas is large. The amount of healthy-state gas contained in defecation gas in advanced cancer is less than that in early cancer. In addition, if the amount of healthy-state gas and the amount of odiferous gas is an intermediate amount between that of cancer patients and that of healthy people, the amount is within a gray zone, that is, it is thought that the gray zone is a state before having disease. Accordingly, the present inventors have thought on the basis of knowledge described above that if the amount of healthy-state gas of a test subject and the amount of odiferous gas, are measured, it is possible to improve determination accuracy of health condition on the basis of a correlation between the amounts.

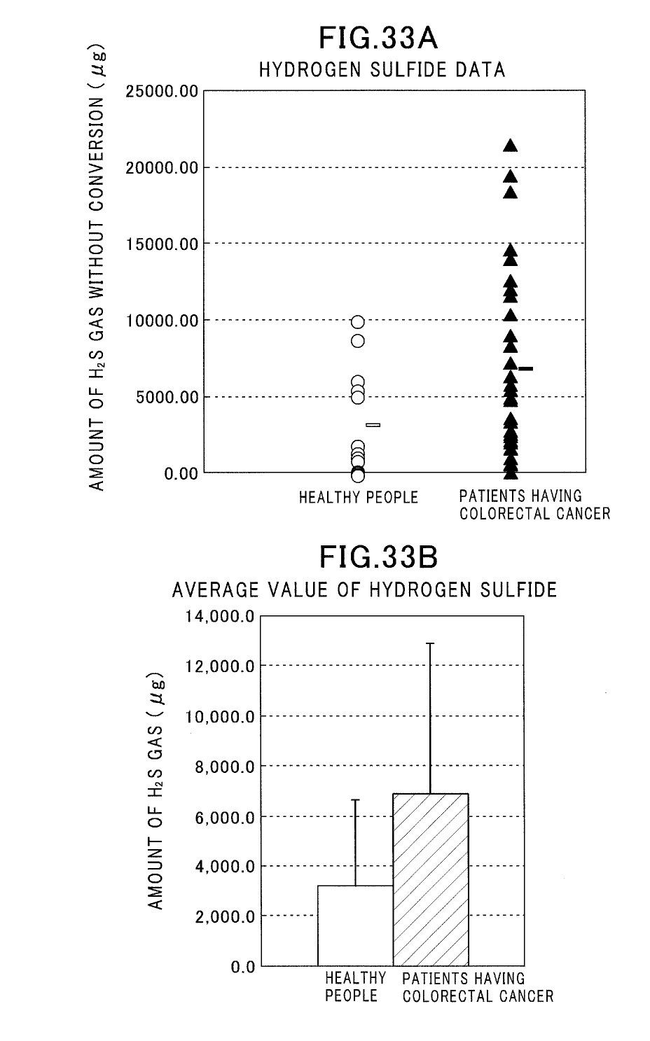

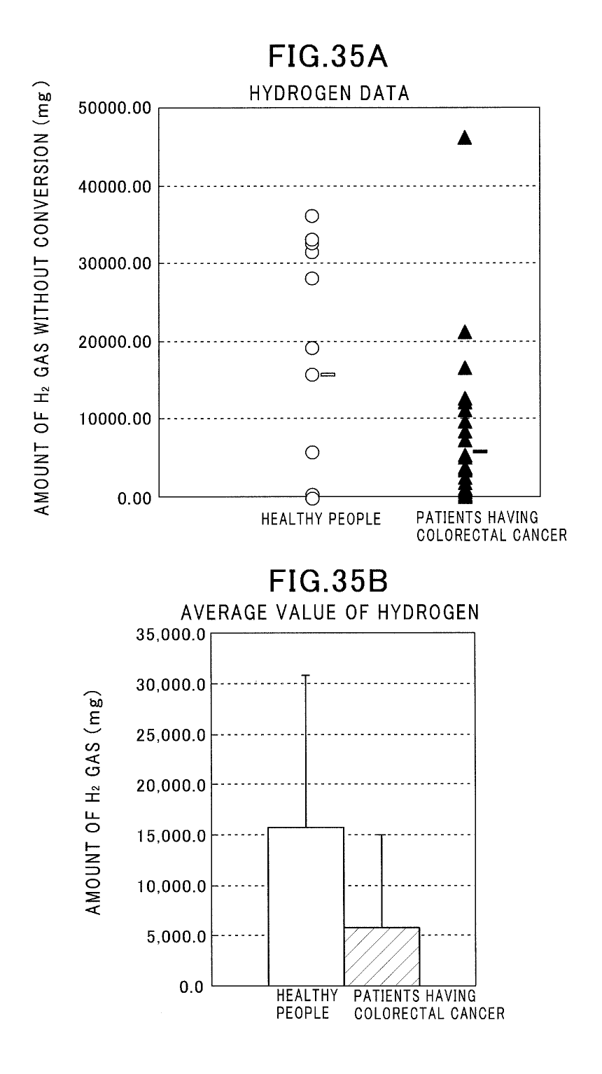

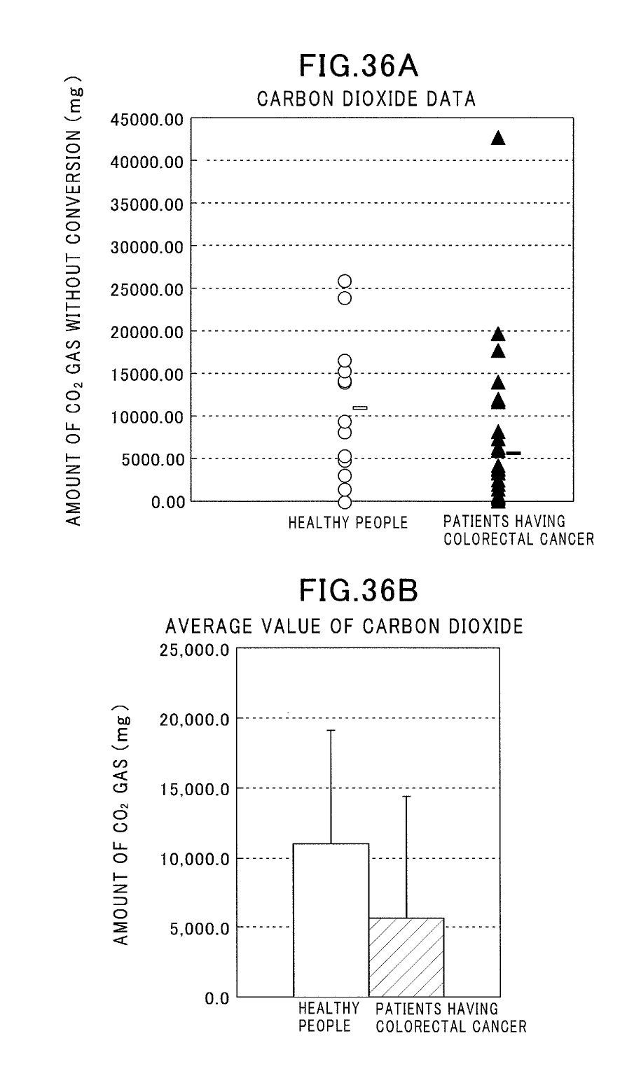

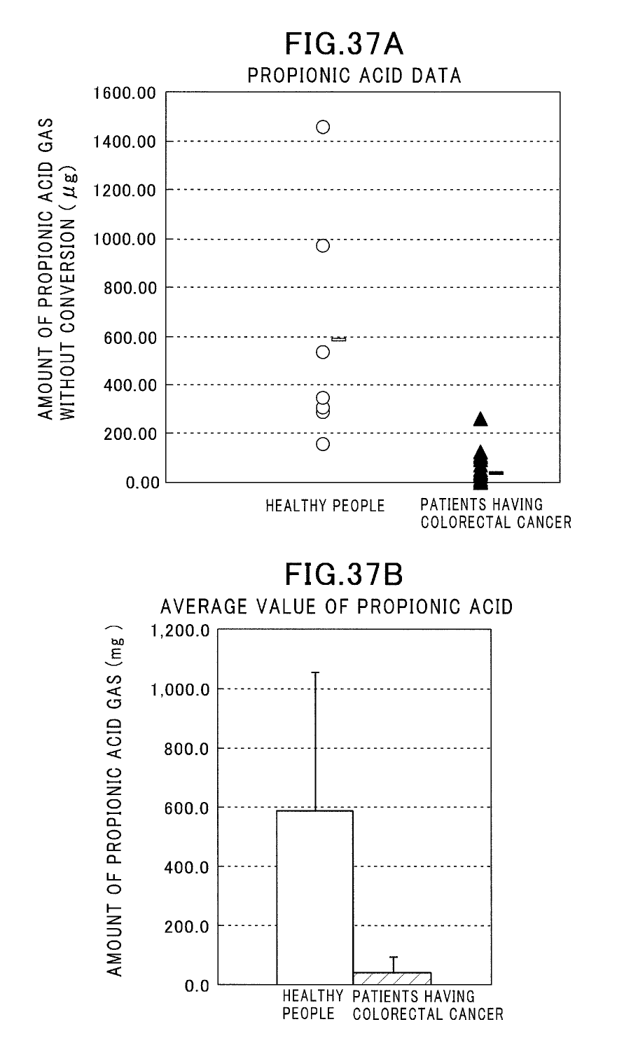

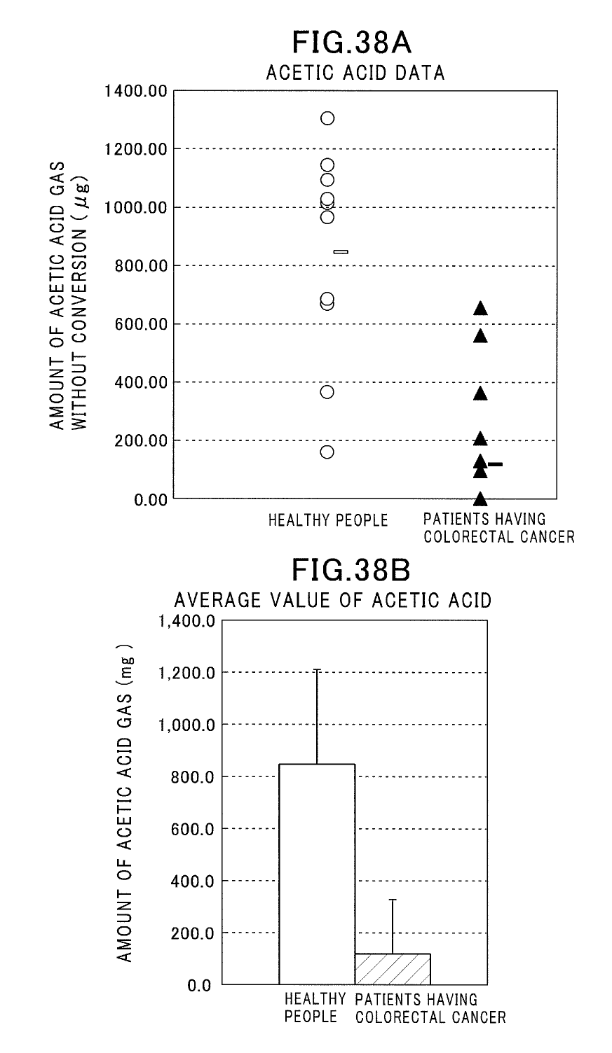

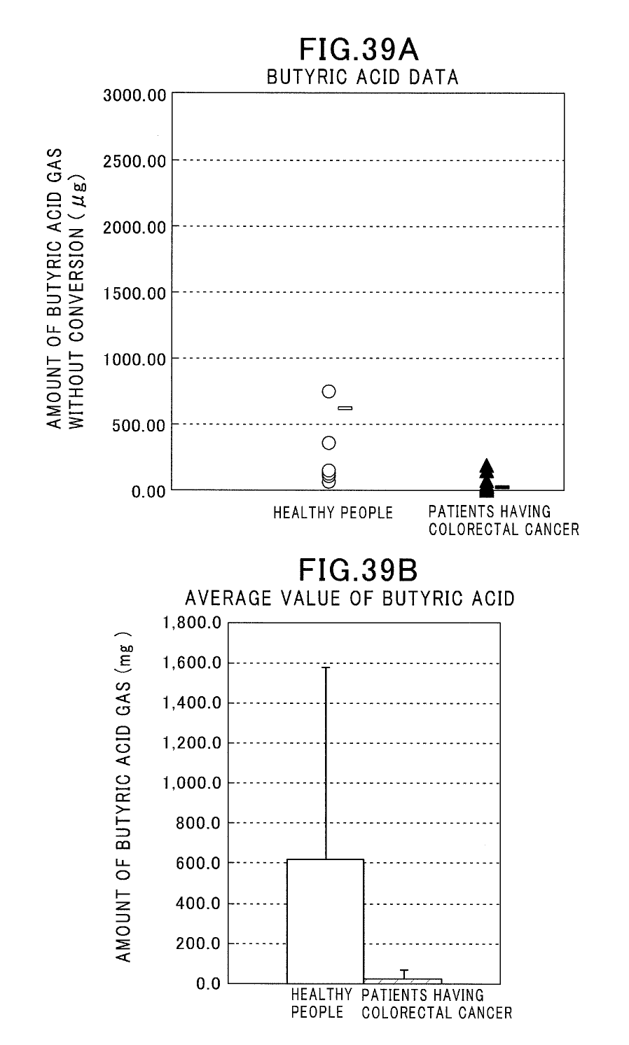

In addition, FIGS. 33 to 39 show measurement data on the amount of various kinds of gas contained in defecation gas, in which healthy people and colorectal cancer patients (including advanced cancer, and early cancer) are compared.

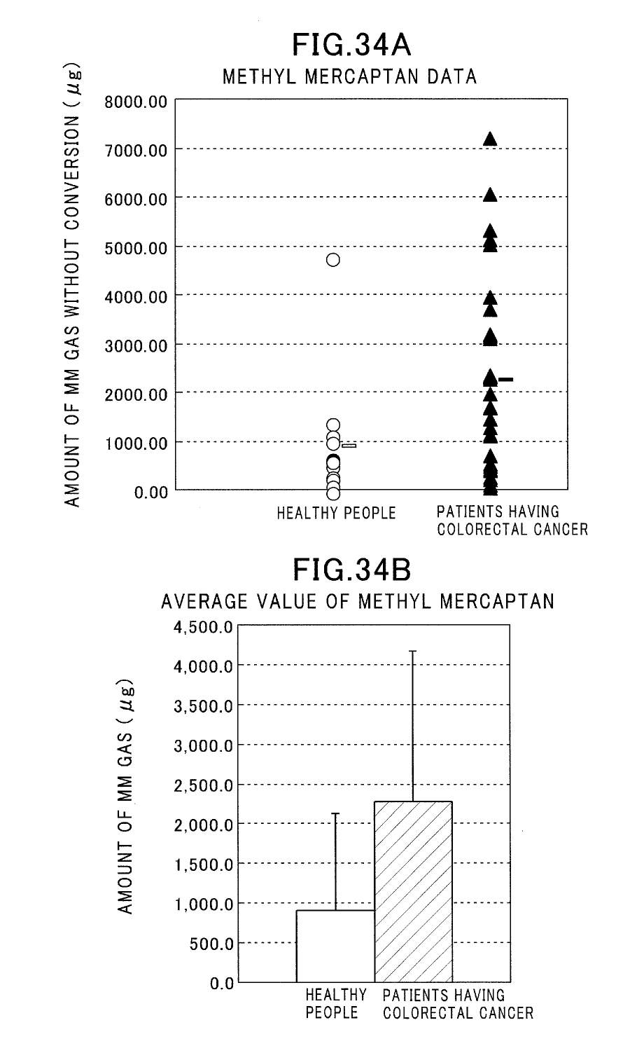

FIGS. 33A and 33B show the amount of hydrogen sulfide contained in defecation gas, in which healthy people and colorectal cancer patients are compared, and FIGS. 34 to 39 show the amount of methyl mercaptan gas, hydrogen gas, carbon dioxide gas, propionic acid gas, acetic acid gas, and butyric acid gas, respectively, in each of which healthy people and colorectal cancer patients are compared. In each of FIGS. 34 to 39, a portion (a) shows measurement data on the amount of each gas by plotting healthy people with a circular mark, and colorectal cancer patients with a triangular mark. In addition, each of portions (b) shows an average value of each measurement data with a bar graph, and standard deviation of each of the measurement data with a line segment.

As is evident from the measurement data shown in FIGS. 33 to 39, although the amount of various kinds of gas contained in defecation gas greatly varies in both healthy people and colorectal cancer patients, with respect to hydrogen sulfide gas and methyl mercaptan gas of odiferous gas, data indicating a large amount of gas is shown many times in the colorectal cancer patients, but there is little data indicating a large amount of gas in the healthy people. Meanwhile, with respect to hydrogen gas, and carbon dioxide gas, there is data indicating a large amount of gas in the healthy people, and there is little data indicating a large amount of gas in the colorectal cancer patients. In this way, while the amount of odiferous gas contained in defecation gas, indicating a risk of colorectal cancer, is large in the colorectal cancer patients, and small in the healthy people, the amount of hydrogen gas and carbon dioxide gas of healthy-state gas is large in the healthy people, and small in the colorectal cancer patient. Accordingly, magnitude relation between the amount of odiferous gas and the amount of healthy-state gas is reversed between the healthy people and the colorectal cancer patient. Although it is difficult to sufficiently measure physical condition of a test subject by using the measurement data acquired by one measurement of the amount of odiferous gas and healthy-state gas, the measurement data shows that if relation between odiferous gas and healthy-state gas is continuously measured multiple times for a predetermined period, it is possible to reliably measure physical condition of a test subject.

When measured defecation gas, the present inventors found that the amount of defecation gas discharged with the first excretory act was large, and a large amount of odiferous gas was also contained in a case where an excretory act was performed multiple times during one defecation (action of discharging a fart once or a stool once). Thus, in the present embodiment, health condition of a test subject is analyzed on the basis of defecation gas acquired first to accurately measure odiferous gas in trace amount. Accordingly, although measurement may be affected by a stool and a fart discharged by the first excretory act when the amount of gas discharged during the second excretory act or later is measured, this influence can be reduced.

The biological information measurement system 1 of the present embodiment is formed on the basis of the measurement principle described above. In the description below, odiferous gas includes methyl mercaptan gas of odiferous gas containing a sulfur component, and odiferous gas, such as hydrogen sulfide other than the methyl mercaptan, methyl mercaptan, acetic acid, trimethylamine, and ammonia.

Next, a specific configuration of the biological information measurement system 1 of the present embodiment will be described in detail.

As shown in FIG. 1, the device 10 on a test subject side in the biological information measurement system 1 is attached to the flush toilet 2 in the toilet installation room R, and a part thereof is assembled into a seat 4 with a function of cleaning anus. The seat 4 with a function of cleaning anus is provided with a suction device 18 that sucks gas in a bowl 2a of the flush toilet 2, as the measuring device 6, and a gas detector 20 that detects a specific component of the gas sucked. The suction device 18 shares a part of a function with a deodorizing device that is usually assembled in the seat 4 with a function of cleaning anus. Gas sucked by the suction device 18 is deodorized by the deodorizing device, and then is returned into the bowl 2a. Each of devices assembled in the seat 4, such as the suction device 18, and the gas detector 20, is controlled by a built-in control device 22 provided on a seat side (refer to FIG. 2).

As shown in FIG. 2, the device 10 on a test subject side is composed of the measuring device 6 assembled in the seat 4, and a data analyzer 60 built in the remote control 8.

The measuring device 6 includes a CPU 22a, and the control device 22 provided with a storage device 22b. The control device 22 is connected to a hydrogen gas sensor 24, an odiferous gas sensor 26, a carbon dioxide sensor 28, a humidity sensor 30, a temperature sensor 32, an entrance detection sensor 34, a seating detection sensor 36, a defecation/urination detection sensor 38, a toilet lid opening/closing device 40, a nozzle driving device 42, a nozzle cleaning device 44, a toilet cleaning device 46, a toilet disinfection device 48, an aromatic sprayer 50 of an aromatic injection device, a deodorizing air supply device 52, the suction device 18, a sensor heater 54, a transmitter-receiver 56, and a duct cleaner 58. As described later, the hydrogen gas sensor and the odiferous gas sensor may be formed into an integrated sensor.

The temperature sensor 32 measures temperature of a detecting portion of the odiferous gas sensor 26, and the like. The humidity sensor 30 measures humidity of gas sucked from the inside of the bowl 2a. Sensitivity of these sensors slightly varies depending on temperature of the detecting portion. Likewise, humidity change due to urination, and the like, affects sensitivity of the sensors. In the present embodiment, the amount of odiferous gas is very small in amount, so that the CPU 22a on a toilet side controls the sensor heater 54 described later, and a humidity adjuster 59 (refer to FIG. 3) to allow sensor temperature and suction humidity of the sensors 30 and 32 to be accurately maintained within a predetermined range, depending on temperature and humidity measured by the sensors 30 and 32, respectively. As a result, the sensor temperature and the suction humidity are adjusted to a predetermined temperature and humidity environment to enable gas in trace amount to be accurately and steady measured. These sensors and devices are not always required, and it is desirable to provide them to improve accuracy.

The entrance detection sensor 34 is an infrared ray sensor, for example, and detects entrance and leaving of a test subject into and from the toilet installation room R.

The seating detection sensor 36 is an infrared ray sensor, a pressure sensor, or the like, for example, and detects whether a test subject sits on the seat 4 or not.

In the present embodiment, the defecation/urination detection sensor 38 is composed of a microwave sensor, and is configured to detect a state of defecation, such as whether a test subject has discharged urine or a stool, whether a stool floats or sinks in seal water, and whether a stool is a diarrhea state or not. Alternatively, the defecation/urination detection sensor 38 may be composed of a CCD, and a water level sensor that measures transition of seal water.

The toilet lid opening/closing device 40 is provided to open and close a toilet lid on the basis of a detection signal of the entrance detection sensor 34, and the like, and according to a situation.

The nozzle driving device 42 is used to clean anus, and cleans anus of a test subject after defecation. The nozzle driving device 42 is configured to drive a nozzle to clean the flush toilet 2.

The nozzle cleaning device 44 cleans a nozzle of the nozzle driving device 42, and in the present embodiment, is configured to create hypochlorous acid from tap water to clean the nozzle with the hypochlorous acid created.

The toilet cleaning device 46 discharges water or tap water stored in a cleaning water tank (not shown) into a toilet to clean the inside of the bowl 2a of the flush toilet 2. Although the toilet cleaning device 46 is usually operated by a test subject while operating the remote control 8 to clean the inside of the bowl 2a, as described later, it is automatically operated by the control device 22 according to a situation.

The toilet disinfection device 48, for example, creates disinfecting water, such as hypochlorous acid water, from tap water, and sprays the disinfecting water created onto the bowl 2a of the flush toilet 2 to disinfect the bowl 2a.

The aromatic sprayer 50 sprays a predetermined aromatic into the toilet installation room R to prevent a test subject from spraying an arbitrary aromatic into the toilet installation room R to prevent an odor component that may be a disturbance with respect to measurement from being sprayed. Providing the aromatic sprayer 50 enables the predetermined aromatic in predetermined amount that does not affect measurement to be sprayed in a predetermined period according to a situation, and then the biological information measurement system 1 is able to recognize that the aromatic is sprayed. Accordingly, a disturbance with respect to measurement of physical condition is reduced to stabilize analysis results, so that the aromatic sprayer 50 serves as output result stabilizing means.

The suction device 18 is provided with a fan for sucking gas in the bowl 2a of the flush toilet 2, and the sucked gas is deodorized by a deodorant filter after flowing through a detecting portion of the odiferous gas sensor 26, and the like. Details of a configuration of the suction device 18 will be described later.

The deodorizing air supply device 52 discharges air that is deodorized after being sucked by suction device 18 into the bowl 2a.

The sensor heater 54 is provided to apply thermal activation to a detecting portion of the odiferous gas sensor 26, and the like. Maintaining a detecting portion at a predetermined temperature enables each sensor to accurately detect a predetermined gas component.

The duct cleaner 58 is provided to clean the inside of a duct 18a attached to the suction device 18 with hypochlorous acid acquired by electrolysis of tap water, or the like, for example.

In the present embodiment shown in FIG. 1, the suction device 18, the deodorizing air supply device 52, and the duct cleaner 58, are integrally formed into the deodorizing device. That is, the suction device 18 sucks gas in the bowl 2a into the duct 18a so that a deodorant filter 78 (refer to FIG. 3) applies deodorizing processing to the sucked gas, and then the gas to which the deodorizing processing is applied is discharged into the bowl 2a again. As a result, it is prevented that gas, to which the odiferous gas sensor 26 is sensitive, flows into the bowl 2a from the outside to change gas components in the bowl 2a during defecation of a test subject by a factor other than defecation gas discharged by the test subject. Thus, the deodorizing device provided with the deodorant filter 78, and the deodorizing air supply device 52, serve as output result stabilizing means. Alternatively, as a variation, the present invention may be configured to provide a gas supply device for measurement (not shown) that allows gas that is insensitive to each gas sensor to flow into the bowl 2a so as to allow gas for measurement with the same amount of gas sucked by the suction device 18 to flow into the bowl 2a. In this case, the gas supply device for measurement (not shown) serves as output result stabilizing means for stabilizing analysis results.

Next, as shown in FIG. 2, the remote control 8 is provided with the built-in data analyzer 60 to which a test subject identification device 62, an input device 64, a transmitter-receiver 66 which is a communication device, a display device 68, and a speaker 70, are connected. In the present embodiment, the transmitter-receiver 66, the display device 68, and the speaker 70, serve as an output device that outputs analysis results by the data analyzer 60. The data analyzer 60 is composed of a CPU, a storage device, a program for operating the CPU and the storage device, and the like, and the storage device is provided with a database.

In the present embodiment, the input device 64 and the display device 68 are configured as a touch panel to accept various kinds of input, such as identification information on a test subject, including a name of the test subject, and the like, as well as to display a variety of information items, such as measurement results of physical condition.

The speaker 70 is configured to output various kinds of alarm, message, and the like, issued by the biological information measurement system 1.

In the test subject identification device 62, identification information on a test subject, including a name of the test subject, and the like, is previously registered. When a test subject uses the biological information measurement system 1, names of registered test subjects are displayed in the touch panel, and then the test subject selects his or her own name.

The transmitter-receiver 66 on a remote control 8 side is communicatively connected to the server 12 through a network. The terminal 14 for a test subject is composed of a device capable of displaying data received by a smartphone, a tablet PC, a PC, or the like, for example.

The server 12 includes a defecation gas database. The defecation gas database records measurement data including the amount of odiferous gas and healthy-state gas in each excretory act, and reliability data, along with a measurement date and time, by being associated with identification information on each test subject using the biological information measurement system 1. The server 12 also stores a diagnosis table, and includes a data analysis circuit.

In addition, the server 12 is connected to the medical facility terminal 16 installed in a hospital, a health organization, and the like, through a network. The medical facility terminal 16 is composed of a PC, for example, to enable data recorded in the database of the server 12 to be browsed. Input and output of information to and from the server 12 will be described later.

Subsequently, with reference to FIG. 3, a configuration of the gas detector 20 built in the seat 4 will be described.

First, in the biological information measurement system 1 of the present embodiment, a semiconductor gas sensor is used in the gas detector 20 as a gas sensor to detect odiferous gas and hydrogen gas. In addition, a solid electrolyte type sensor is used in the gas detector 20 to detect carbon dioxide.

The semiconductor gas sensor includes a detecting portion composed of a metal oxide film containing tin oxide, and the like. If the detecting portion is exposed to reducing gas while being heated at a few hundreds degrees, oxidation-reduction reaction occurs between oxygen adsorbed on a surface of the detecting portion and the reducing gas. The semiconductor gas sensor electrically detects change in resistance of the detecting portion by the oxidation-reduction reaction to enable reducing gas to be detected. Reducing gas that a semiconductor gas sensor can detect includes hydrogen gas, and odiferous gas. In the present embodiment, although a semiconductor gas sensor is used in both a sensor for detecting odiferous gas, and a sensor for detecting hydrogen gas, material components of each of detecting portions of the respective sensors is adjusted so that a detecting portion used in the odiferous gas sensor reacts strongly to odiferous gas, and a detecting portion used in the hydrogen gas sensor reacts strongly to hydrogen gas.

In this way, although the present embodiment uses a “semiconductor gas sensor” as an “odiferous gas sensor”, as described above, the “semiconductor gas sensor” is a general type that is sensitive not only to methyl mercaptan gas of a detection object, but also widely to odiferous gas other than that. In addition, as described later, although a solid electrolyte sensor is available for an “odiferous gas sensor”, as with a semiconductor gas sensor, a general type of a solid electrolyte sensor, sensitive to methyl mercaptan gas as well as widely to another odiferous gas other than the methyl mercaptan, may be used. That is, it is very difficult to manufacture a gas sensor that is sensitive only to methyl mercaptan gas, and even if the gas sensor can be manufactured, the gas sensor becomes very large in size and expensive. If this kind of large and expensive gas sensor is used, the gas sensor is feasible for a medical device used in advanced clinical examination, but it is impossible to manufacture a biological information measurement system at a cost enabling the system to be sold as a consumer product. The biological information measurement system of the present embodiment uses a simple and general gas sensor that is sensitive also to another odiferous gas other than methyl mercaptan gas of a detection object, as the “odiferous gas sensor”, to be feasible as a consumer product. As described above, although the gas sensor used in the present embodiment is sensitive to methyl mercaptan gas, as well as to odiferous gas other than the methyl mercaptan gas, the gas sensor is referred to as an “odiferous gas sensor” in the present specification, for convenience. The “odiferous gas sensor” used in the present embodiment is sensitive to odiferous gas that representatively includes methyl mercaptan gas, hydrogen sulfide gas, ammonia gas, and alcoholic gas.

Although the “odiferous gas sensor” used in the biological information measurement system 1 of the present embodiment is sensitive to methyl mercaptan gas of an object, as well as to odiferous gas other than that, a variety of devices enable even this kind of gas sensor to be used for measurement with necessary and sufficient accuracy as a consumer product. Specifically, the devices include a device to improve a measurement environment in a space of a toilet installation room where a variety of odiferous gases exist, a device for data processing of extracting data on defecation gas by assuming defecation act of a test subject from a detection signal provided by a gas sensor, a device to prevent an excessive mental burden from being applied to a test subject even if detection data with a large error is acquired, and the like. Each of the devices will be described later in detail.

Although the present embodiment describes a case where a semiconductor gas sensor is used for a sensor for detecting odiferous gas and hydrogen gas, a solid electrolyte sensor is also available instead of the semiconductor gas sensor. The solid electrolyte sensor, for example, detects gas on the basis of the amount of ions that penetrates its solid electrolyte, such as stabilized zirconia, while the solid electrolyte is heated. Gas which can be detected by the solid electrolyte sensor includes hydrogen gas, and odiferous gas. In the present embodiment, a solid electrolyte sensor is used as a sensor for detecting carbon dioxide. A carbon dioxide sensor is not limited to the above sensors, and an infrared sensor or the like may be available. The sensor for detecting carbon dioxide may be eliminated.

As shown in FIG. 3, in the present embodiment, the gas detector 20 is arranged inside the suction device 18.

The suction device 18 includes the duct 18a directed downward, an air intake passage 18b directed substantially in a horizontal direction, and a suction fan 18c arranged downstream of the air intake passage 18b. In the duct 18a, the duct cleaner 58, and the humidity adjuster 59, are provided.

The gas detector 20 includes a filter 72 arranged inside the air intake passage 18b, the odiferous gas sensor 26, the hydrogen gas sensor 24, and the carbon dioxide sensor 28. As shown in FIG. 3, the filter 72 is arranged so as to traverse the air intake passage 18b, and the odiferous gas sensor 26, the hydrogen gas sensor 24, and the carbon dioxide sensor 28, are juxtaposed downstream of the filter 72.

In addition, the deodorant filter 78 is provided downstream of the odiferous gas sensor 26, so that the suction device 18 also serves as a deodorizing device by allowing the deodorant filter 78 to deodorize sucked gas.

Further, the humidity adjuster 59 is provided downstream of the deodorant filter 78. The humidity adjuster 59 is filled with a desiccant, and if it is required to reduce humidity in the bowl 2a, moisture is removed from air circulating in the bowl 2a by switching a flow channel so that the air passing through the deodorant filter 78 passes through the filled desiccant. Accordingly, the humidity in the bowl 2a is maintained at a proper value to maintain detection sensitivity of each gas sensor at an almost constant level.

The suction fan 18c sucks stink gas containing odiferous gas, and the like, in the bowl 2a of the flush toilet 2, at a constant speed to deodorize the stink gas, and then returns the gas into the bowl 2a. The duct 18a for deodorization opens in the bowl 2a while its suction port is directed downward to prevent a splash of urine or the like from entering the inside of the duct 18a. Molecular weight of odiferous gas, such as methyl mercaptan, and of hydrogen gas, is small enough to allow the gases to rise immediately after defecation. In contrast, in the present embodiment, odiferous gas and hydrogen gas discharged is sucked by suction fan 18c through an inlet of the duct 18a, opening in the bowl 2a, so that it is possible to reliably guide the gases into the gas detector 20. In this way, the suction device 18 is operated before a test subject starts defecation, and brings gas at a constant flow velocity into contact with each gas sensor during defecation of the test subject. Accordingly, it is possible to acquire a steady measurement value.

The filter 72 does not have a deodorizing function, and is configured so as to allow odiferous gas, hydrogen, and carbon dioxide to pass therethrough, as well as to prevent foreign material, such as urine, and a cleaner from passing therethrough. For this kind of filter 72, a member for mechanically collecting the foreign material without using chemical reaction, such as a fine net-like member, is available. Accordingly, it is possible to prevent the odiferous gas sensor 26, the hydrogen gas sensor 24, and the carbon dioxide sensor 28, from being contaminated by a urinary calculus, or the like.

The sensor heater 54 is provided upstream of each gas sensor, and downstream of the filter 72. As described above, the odiferous gas sensor 26 and the hydrogen gas sensor 24, each of which is a semiconductor gas sensor, are capable of detecting hydrogen and odiferous gases while each of their detecting portions is heated to a predetermined temperature. The sensor heater 54 is provided to heat the detecting portions of the odiferous gas sensor 26 and the hydrogen gas sensor 24. The carbon dioxide sensor 28 is also required to heat its solid electrolyte to a predetermined temperature, so that the sensor heater 54 is provided. The sensor heater 54 also serves as a stink removing device for thermally removing stink gas components attached to each of the sensors. Even if a solid electrolyte sensor is used as the odiferous gas sensor, and the hydrogen gas sensor, it is required to provide a sensor heater for heating a detecting portion.

The sensor heater 54 also serves as means for removing a deposit attached to each sensor. Although foreign material is removed from gas passing through the filter 72, the sucked gas contains various stink gas components. Such stink gas components are attached to each gas sensor, and may cause noise when odiferous gas in trace amount is measured. In contrast, the sensor heater 54 heats a detecting portion of a sensor to enable stink gas attached to the sensor to be thermally removed without providing an additional device. The control device 22 controls the sensor heater 54 before a test subject starts defecation act so as to allow temperature of each gas sensor to be constant. That is, the control device 22 controls the sensor heater 54 so as to prevent temperature of each gas sensor from decreasing due to contact of an air flow. Accordingly, it is possible to maintain sensitivity of each gas sensor at a predetermined value during defecation of a test subject to enable a measurement error of each gas sensor to be reduced.

The deodorant filter 78 is a catalytic filter that adsorbs stink gas, such as odiferous gas. The deodorant filter 78 removes gas, such as odiferous gas, from air, and the air is returned to the bowl 2a. Then, if odiferous gas or the like is contained in the gas returned into the bowl 2a, the odiferous gas or the like flows into the bowl 2a may be sucked through the duct 18a again to be detected by the odiferous gas sensor 26 again. Thus, in the present embodiment, the deodorant filter 78 is arranged downstream of the odiferous gas sensor 26 to reliably remove odor components, such as odiferous gas, from gas returned into the bowl 2a.

If a test subject sits on the seat 4, a portion above the bowl 2a is closed by his or her underwear, or the like. If the inside of the bowl 2a is placed under negative pressure, stink gas components attached to a body, clothes, and the like, of the test subject, may be sucked into the bowl 2a. In the biological information measurement system 1 of the present embodiment, sensitivity of the odiferous gas sensor 26 is set very high to detect only a trace amount of odiferous gas contained in defecation gas, so that even stink gas components attached to a body, clothes, and the like, of a test subject, may be a disturbance with respect to measurement. In contrast, in the present embodiment, gas after deodorized is returned into the bowl 2a, so that the inside of the bowl 2a is not placed under negative pressure to enable gas components attached to a body, clothes, and the like, of a test subject, to be prevented from being sucked into the bowl 2a.

Here, the semiconductor gas sensor used as the odiferous gas sensor 26 detects not only odiferous gas but also hydrogen. Thus, it is required to separate influence of hydrogen gas from detection data acquired by the semiconductor gas sensor. In the present embodiment, as a hydrogen separation mechanism for separating this kind of influence of hydrogen gas, in the gas detector 20, a detection value of hydrogen gas detected by the hydrogen gas sensor 24 is subtracted from a detection value of odiferous gas detected by the semiconductor gas sensor to separate influence of hydrogen gas so that the calculated value is outputted as a detection value of the odiferous gas sensor 26. A configuration that is composed of this kind of hydrogen separation mechanism, the semiconductor gas sensor, and the hydrogen gas sensor 24, to output a detection value corresponding to the amount of odiferous gas and hydrogen gas, is referred to as a detection value output mechanism. Calculation processing of subtracting a detection value of hydrogen gas detected by the hydrogen gas sensor 24 from a detection value of odiferous gas detected by the semiconductor gas sensor described above may be performed in the data analyzer 60, or the like. Although the present embodiment describes the hydrogen separation mechanism for separating influence of hydrogen gas from detection data acquired by the semiconductor gas sensor, it is also possible to separate influence of methane from detection data acquired by the semiconductor gas sensor by providing a methane sensor for detecting methane. A semiconductor gas sensor with a detecting portion composed of material components adjusted so as to strongly react to methane may be used as the methane gas sensor.

Many people have no methane producer that produces methane in their intestine, or have very low amount thereof if existing, so that many people have a very low amount of methane contained in defecation gas. Thus, in the present embodiment, the hydrogen sensor 24 and the carbon dioxide sensor 26 are provided as a healthy-state gas sensor. However, a few people have a very large amount of methane producer in their intestines. Defecation gas of people having a very large amount of intestinal methane producer as described above contains a large amount of produced methane, but contains a low amount of produced hydrogen. Thus, if only the hydrogen sensor 24 and the carbon dioxide sensor 26 are provided, defecation gas of people having a very large amount of intestinal methane producer is unfavorably determined that there is a small amount of discharged healthy-state gas. In the present embodiment, although the hydrogen sensor 24 and the carbon dioxide sensor 26 are provided as a healthy-state gas sensor to fit with many people, a methane gas sensor instead of the hydrogen sensor 24 may be provided to fit with people having a large amount of methane gas. In addition, it is more preferable to provide the methane gas sensor in addition to the hydrogen sensor 24 and the carbon dioxide sensor 26 in advance to be able to correspond to any test subject.

As described above, defecation gas contains a large amount of hydrogen, and the semiconductor gas sensor detects not only odiferous gas but also hydrogen. For that, influence of hydrogen can be separated by subtracting the amount of hydrogen gas detected by the hydrogen gas sensor 24 from the amount of gas detected by the odiferous gas sensor 26 of a semiconductor gas sensor, so that it is possible to accurately measure the amount of odiferous gas.

In addition, hydrogen gas contained in defecation gas has very small molecular weight as compared with air to be easily released from the bowl 2a. For that, in the present embodiment, defecation gas is sucked by the fan 18c of the suction device 18 to enable defecation gas containing hydrogen gas to be reliably collected.

If sucked defecation gas is returned into the bowl 2a as it is, measurement accuracy by the odiferous gas sensor 26 decreases. In contrast, in the present embodiment, sucked defecation gas is deodorized by the deodorant filter 78 to be returned into the bowl to enable the amount of odiferous gas and hydrogen to be accurately measured. In addition, although the deodorant filter 78 as above is required to be arranged downstream of each sensor, if the deodorant filter 78 as above is provided downstream of each sensor, the sensor may be directly contaminated by foreign material. In contrast, in the present embodiment, the filter 72 without a deodorizing function is provided upstream of a sensor to enable contamination of the sensor by foreign material to be reduced without affecting measurement of odor components.

If gas is sucked into the bowl 2a, pressure in the bowl 2a decreases, and thus stink gas components attached to a body and clothes of a test subject may flow into the bowl 2a. In contrast, in the present embodiment, air after odor components have been deodorized is returned into the bowl 2a, so that stink gas components attached to a body and clothes of a test subject are prevented from flowing into the bowl 2a to enable accurate measurement. A configuration in which air after being deodorized to remove odor components is returned into the bowl 2a is not essential.

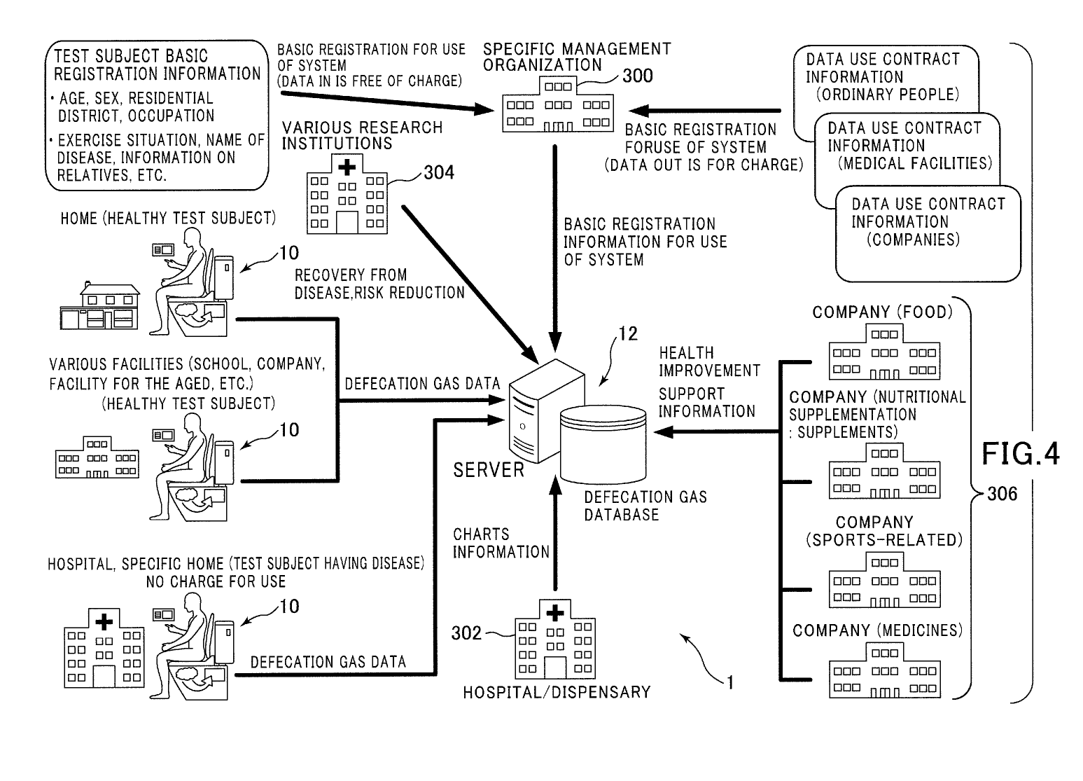

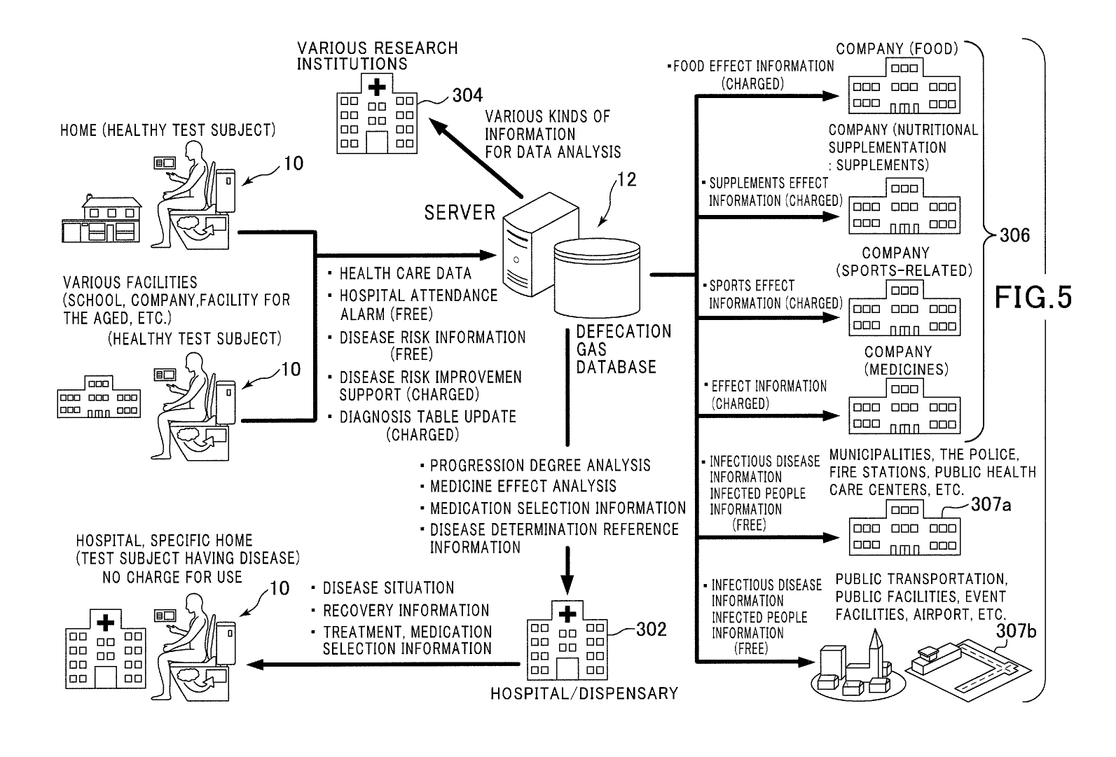

Next, with reference to FIGS. 4 and 5, a flow of information between test subject side devices and the server, and a flow of information between the server and a hospital, companies and the like in the biological information measurement system 1 according to the first embodiment of the present invention will be described.

FIG. 4 shows a flow of information sent to the server from the test subject side devices, a hospital, companies and the like. FIG. 5 shows a flow of information sent from the server to the test subject side devices, the hospital, the companies and the like.

As shown in FIG. 4, the devices 10 on a test subject side which are purchased by respective test subjects, and are respectively installed in residences and the like are configured to be capable of performing data communication with the server 12 bidirectionally, and each time the test subjects perform defecation every day, detection data of the defecation gas and the like are transmitted to the server 12, and accumulated. Further, the device 10 on a test subject can be also installed in a medical facility (a hospital) 302, and various facilities such as schools, homes for the aged and nursing facilities. Meanwhile, the server 12 is managed by a specific management organization 300 that manages the biological information measurement system 1 according to the present embodiment. A user (a test subject) who has purchased the device 10 on a test subject side registers age, sex, residential district, occupation, living environment, and living habits such as exercise, medical history of himself or herself and medical histories of relatives in the server 12 via the specific management organization 300. When the user installs the device 10 on a test subject side in his or her residence, the information on family members who live in the residence and use the device 10 on a test subject side is also registered. Further, when the devices 10 on a test subject side are installed in a medical facility and a nursing facility, information on residents and inpatients using the devices 10 on a test subject side is registered in the server 12. When an inpatient and a resident who use the devices 10 on a test subject side in a medical facility and a nursing facility have also used the devices 10 on a test subject side at home, it is preferable that the test subjects are registered so that detection data of defecation gas acquired at home can be associated with detection data acquired in the medical facility and the like.

Meanwhile, various research institutions 304, the medial facility 302, various companies 306 and the like which desire to use information on physical conditions of a large number of test subjects accumulated in the server 12 enter into a data use agreement with the specific management organization 300, and register the information in the server 12.

The various research institutions 304 can make use of the information on defecation gas of a large number of test subjects accumulated in the server 12, and the information on diseases the test subjects have been affected and the like for prevention of and treatment for the diseases. The various companies 306 can transmit information on services provided by themselves to corresponding test subjects in accordance with physical conditions of a large number of test subjects and the diseases affecting the test subjects, and the like accumulated in the server 12.

Further, when a test subject using the device 10 on a test subject side at home or the like has undergone diagnosis in the medical facility 302, the medical facility 302 transmits information on a condition of the disease of the test subject and the like to the server 12. At this time, the information of the condition of the disease and the like transmitted to the server 12 is transmitted in such a format that can associate the patient (test subject) who has undergone diagnosis in the medical facility 302 with the detection data of the test subject accumulated in the server 12.

Next, with reference to FIG. 5, information that is provided to each place from the server 12 will be described.

First, to the respective devices 10 on a test subject side, physical condition states of test subjects analyzed in the server 12 on the basis of the detection data transmitted from the respective devices 10 on a test subject side are transmitted. The information transmitted to the test subjects from the server 12 can be transmitted to the remote controls 8 (FIG. 1) of the devices 10 on a test subject side, and terminals 14 for test subjects such as smartphones used by the test subjects. Information on time-dependent changes in the stages of health of test subjects and risks of the test subjects being affected by specific diseases in a future are provided to the test subjects. Further, information on medicines suited to the test subjects, supplements, health food, and sports clubs, useful for improvement of the states of health of test subjects, which are provided by the various companies 306 registered in the server 12, is also provided to the test subjects. Details of the information provided to the test subjects will be described later.

To the medical facility 302, information on time-dependent changes in the symptoms of the test subjects undergoing diagnosis in the medical facility 302, the effect of medication to the test subject, the effect of a specific medicine to the other test subjects affected by the same disease and the like is provided. Accordingly, doctors of the medical facility 302 can grasp the symptoms of the test subjects more accurately, perform more suitable medication, and set treatment plans.

Further, to the various research institutions 304, information on transition of the symptoms of many test subjects suffering from a specific disease, an effect of medication and the like is provided, with information on detection data of defecation gas of the test subjects. Accordingly, researchers in the various research institutions 304 can utilize the information of a relationship between the amounts of respective components in defecation gas and a specific disease, an effect of a medicine to a specific disease, and the like in research.

To the various companies 306, information on subsequent changes in the physical condition, of the test subjects utilizing commodities and services provided by the various companies is provided. Accordingly, each of the companies can make use of the provided information for improvement of qualities of the commodities and the services provided by each of the companies.

Further, part of predetermined information accumulated in the server 12 is also provided to public institutions 307a such as municipalities, the police, fire departments, and public health care centers, and public facilities 307b such as airports, railway stations, harbor, and event facilities. Utilization of the information in these institutions and facilities will be described later.

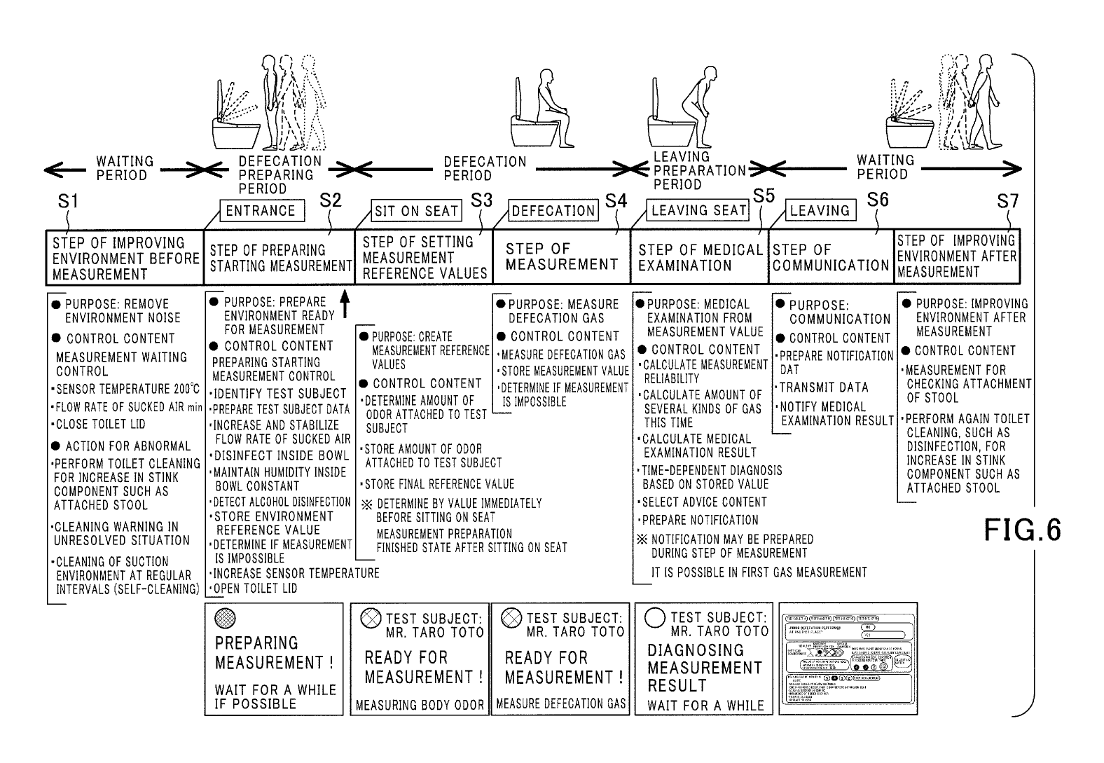

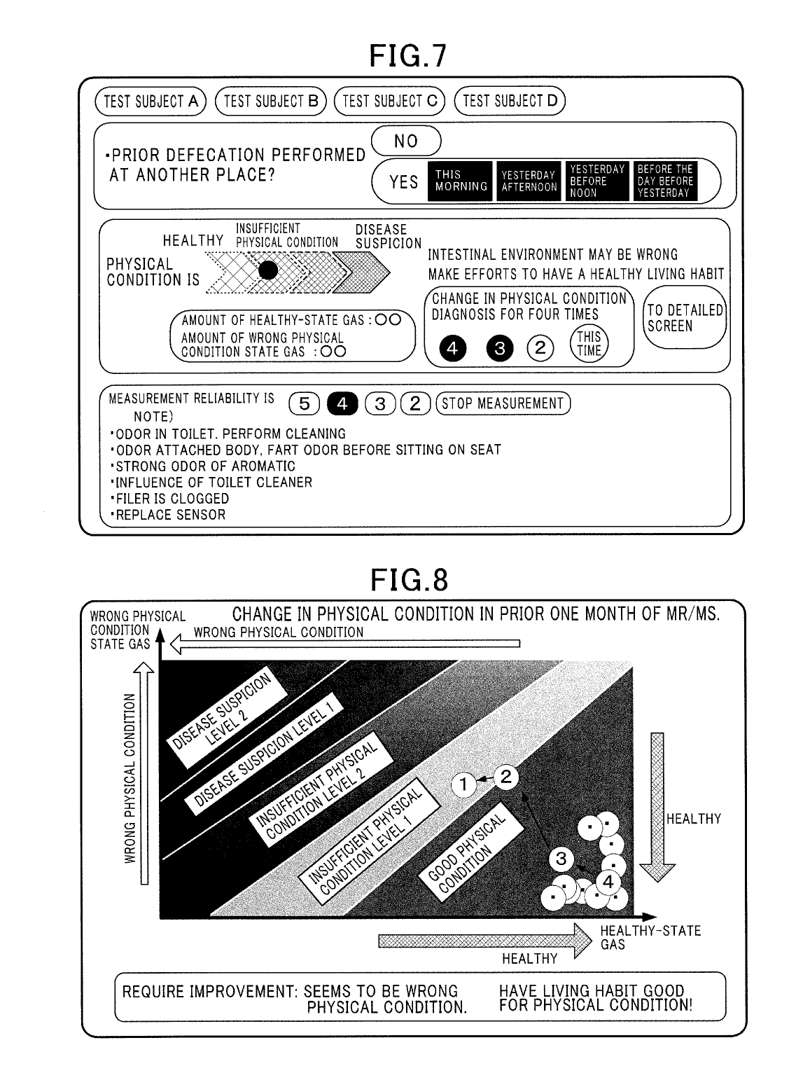

Next, with reference to FIGS. 6 and 7, a flow of measurement of physical condition by the biological information measurement system 1 in accordance with the first embodiment of the present invention will be described.

FIG. 6 describes a flow of measurement of physical condition, and an upper section shows each step of the measurement of physical condition, as well as a lower section shows an example of screens to be displayed in a display device of a remote control in each step. FIG. 7 shows an example of the screens to be displayed in the display device of the remote control.

The biological information measurement system 1 of the present embodiment analyzes physical condition including determination of cancer on the basis of a correlation between odiferous gas and healthy-state gas, in defecation gas discharged by a test subject during defecation. In each test subject side device, it is preferable that an analysis result is displayed during defecation, or in a short time until leaving a toilet installation room after one defecation period has been finished. However, if analysis is performed in a short time, analysis accuracy may decrease. It is difficult that the suction device 18 sucks the whole of defecation gas discharged by a test subject, and a condition where the inside of a toilet or a toilet installation room is very unsanitary, or a measurement environment with a strong aromatic, becomes a disturbance that affects measurement accuracy so that it may decrease. Thus, when physical condition including whether there is a disease or not is notified to a test subject in each test subject side device, in consideration of a mental burden of the test subject, it is devised that not only an absolute amount of odiferous gas having a strong relationship with cancer, but also change in physical condition of a test subject, or change in intestinal conditions, is strongly notified to the test subject, on the basis of time-dependent results acquired by measurement performed during defecation act performed many times for a long time. In addition, also in consideration of a measurement error during each defecation act, in the present embodiment, it is devised that physical condition is notified to a test subject on the basis of measurement results during one defecation act so that the physical condition to be notified to the test subject does not largely changes. The device is based on using characteristics of disease of cancer that develops for a long time, because if the amount of odiferous gas having a strong relationship with cancer is largely changed for a short time, it is not caused by a strong relationship with cancer, but largely caused by a result of a bad living habit or influence of noise, whereby a large change in physical condition may apply unnecessary mental anxiety to the test subject.

In the light of the above matter, in the present embodiment, the device 10 on a test subject side simply analyzes health condition on the basis of measurement results of defecation gas discharged first in one defecation act, or defecation gas discharged during the first excretory act to display an analysis result of the health condition. In contrast, the server 12 is capable of a detailed analysis on the basis of a total amount of gas discharged during one defecation act by comparing it with that of other test subjects, and the like. Then, in the biological information measurement system 1 of the present embodiment, the device 10 on a test subject side installed in the toilet installation room R performs a simple analysis, and the server 12 performs a more detailed analysis.

As shown in FIG. 6, in measurement during one defecation act by the biological information measurement system 1 of the present embodiment, the following steps is performed: step S1 of improving environment before measurement; step S2 of preparing starting measurement; step S3 of setting measurement reference values; step S4 of measurement; step S5 of medical examination; step S6 of communication; and step S7 of improving environment after measurement.

Step S1 of improving environment before measurement is performed before a test subject enters the toilet installation room R. The entrance detection sensor 34 (refer to FIG. 2) detects whether a test subject enters the toilet installation room R, or not.

In step S1 of improving environment before measurement, the control device 22 on a seat side allows the sensor heater 54, the suction device 18, and the toilet lid opening/closing device 40, to switch to a measurement waiting mode to control them. The sensor heater 54 is controlled in the measurement waiting mode on the basis of temperature measured by the temperature sensor 32 so that temperature of a detecting portion of the odiferous gas sensor 26 becomes waiting temperature (such as 200° C.) lower than temperature when measurement is performed. The suction device 18 is controlled in the measurement waiting mode so that a flow rate of sucked air becomes minimum. The toilet lid opening/closing device 40 is controlled in the measurement waiting mode so that a toilet lid is closed.

In step S1 of improving environment before measurement, although the detecting portion of the odiferous gas sensor 26 is at a temperature lower than an optimum temperature because the sensor heater 54 is in the measurement waiting mode, it is possible to measure concentration of odiferous gas. If there is an occurrence source of stink gas in the bowl 2a, such as a case where there is a stool attached to the flush toilet 2, or the like, concentration of gas measured by the odiferous gas sensor 26 becomes a predetermined value or more. The control device 22 allows toilet cleaning to be performed if the concentration of gas measured by the odiferous gas sensor 26 exceeds a predetermined value in step S1 of improving environment before measurement. Specifically, the control device 22 performs as follows: allows the nozzle driving device 42 to discharge cleaning water through a nozzle to clean the bowl 2a; allows the toilet cleaning device 46 to discharge water stored in a cleaning water tank into the bowl 2a to clean the inside of the bowl 2a; or allows the toilet disinfection device 48 to create disinfecting water, such as hypochlorous acid water, from tap water, or the like to spray disinfecting water created onto the bowl 2a to disinfect the bowl 2a.

If the concentration of gas measured by the odiferous gas sensor 26 is a predetermined value or more, the control device 22 also enables the suction device 18 to discharge gas in the bowl 2a to reduce concentration of gas. Gas sucked by the suction device 18 is deodorized by the deodorant filter 78, so that the suction device 18 and the deodorant filter 78 serve as a deodorizing device. The suction device 18 sucks gas while the toilet lid is opened to enable not only the inside of the bowl 2a but also the inside of the toilet installation room R to be deodorized, so that the suction device 18 and the deodorant filter 78 can also serve as a toilet installation room deodorizing device. Preferably, if the suction device 18 and the deodorant filter 78 serve as a deodorizing device, the amount of gas to be sucked by the suction device 18 is increased as compared with when measurement of physical condition is performed during defecation of a test subject.

Alternatively, the control device 22 may be configured so as to be able to control a ventilator (not shown) provided in the toilet installation room R to allow the ventilator to operate to reduce concentration of gas. In this way, concentration of odiferous gas remaining in the bowl 2a is reduced to reduce influence of residual gas noise caused by the gas remaining.

In step S1 of improving environment before measurement, if the amount of gas measured by the odiferous gas sensor 26 is not less than a predetermined value even if the toilet cleaning described above is performed, the control device 22 allows the transmitter-receiver 56 to transmit a cleaning warning command signal. When the transmitter-receiver 66 on the remote control 8 side receives the cleaning warning command signal, the display device 68 or the speaker 70 notifies a test subject that toilet cleaning should be performed.

In addition, in step S1 of improving environment before measurement, the control device 22 allows cleaning of suction environment to be performed at regular intervals. Specifically, the control device 22 allows the duct cleaner 58 to operate to spray cleaning water into the duct 18a of the suction device 18 to clean the duct 18a, and the like. Further, the sensor heater 54 heats each of the hydrogen gas sensor 24, the odiferous gas sensor 26, and the carbon dioxide sensor 28, to a high temperature to burn stink gas components attached to a surface of each of the gas sensors 24, 26, and 28.

Next, when the entrance detection sensor 34 detects entrance of a test subject, the control device 22 transmits a signal of starting step S2 of preparing starting measurement to the transmitter-receiver 66 on the remote control 8 side through the transmitter-receiver 56, and then step S2 of preparing starting measurement is performed in synchronization with the remote control side.

In step S2 of preparing starting measurement, first, the test subject identification device 62 built in the remote control 8 identifies a test subject. Specifically, in the biological information measurement system 1, a resident of a house in which the system is installed is registered, and a registered resident is displayed as a candidate of the test subject. That is, as shown in FIG. 7, buttons of respective candidates, such as a “test subject A”, a “test subject B”, and a “test subject C”, are displayed in an upper portion of the display device 68 of the remote control 8, and then a test subject entering the toilet installation room R presses a button corresponding to oneself to identify the test subject. In addition, the data analyzer 60 built in the remote control 8, with reference to data in a storage device, acquires previous measurement data on personal identification information received by the test subject identification device 62, and a physical condition display table as reference data to be a basis of analysis.

In addition, in step S2 of preparing starting measurement, the data analyzer 60, as shown in FIG. 7, allows a display device to display a message in a second section of its screen, such as: a question about whether previous defecation was performed in the toilet installation room in which this device is installed, such as “Was previous defecation performed in another place?”; and options of answers to the question, such as “Yes (This morning)”, “Yes (Yesterday afternoon)”, “Yes (Yesterday before noon)”, “Before the day before yesterday”, and “No”. Once a test subject answers these questions, the input device 64 of the data analyzer 60 receives defecation history information on the test subject. This kind of defecation history information on elapsed time from previous defecation act of a test subject is stored in a storage device (test subject information storage device) built in the remote control 8, and the test subject information storage device also stores information on a test subject previously registered, such as weight, age, and sex. The defecation history information is transmitted to the server 12 to be recorded in a database of the server 12.

In step S2 of preparing starting measurement, the control device 22 on a toilet side allows the sensor heater 54, the suction device 18, and the toilet lid opening/closing device 40 to switch to a measurement mode. The sensor heater 54 is controlled in the measurement mode on the basis of temperature measured by the temperature sensor 32 so that temperature of a detecting portion of the odiferous gas sensor 26 becomes a first temperature (for example, 400° C.) suitable for measurement. That is, the temperature of the detecting portion of the odiferous gas sensor 26 is kept at a second temperature (for example, 200° C.) lower than the first temperature in the waiting state before the test subject enters the toilet installation room, and when it is detected that the test subject enters the toilet installation room, the control device 22 increases the temperature of the detecting portion to the first temperature before the test subject sits on the seat 4. The suction device 18 is controlled in the measurement mode so that a flow rate of sucked air is increased to the extent that defecation gas does not leak to the outside of the bowl 2a to be constantly maintained at the extent so as not to vary. The toilet lid opening/closing device 40 is controlled in the measurement mode so that a toilet lid is opened.

If concentration of odiferous gas detected by the odiferous gas sensor 26 is high in step S2 of preparing starting measurement, the control device 22 allows the toilet disinfection device 48 to disinfect the inside of the bowl 2a.

In step S2 of preparing starting measurement, if humidity measured by the humidity sensor 30 is unsuitable for measurement of defecation gas by the odiferous gas sensor 26, the control device 22 transmits a signal to the humidity adjuster 59 to control it so that humidity in the bowl becomes a proper value.

In the step of preparing starting measurement, when the seat 4 is cleaned with a sheet or spraying, by using alcoholic disinfectant, the odiferous gas sensor 26 reacts to alcohol to suddenly increase concentration of gas. In this way, if concentration of gas measured by the odiferous gas sensor 26 suddenly increases, the data analyzer 60 allows the display device 68 to display a warning.

The data analyzer 60 stores a measurement value measured by the odiferous gas sensor 26, as an environment reference value of a noise level to be a basis of measurement of defecation gas. The data analyzer 60 then determines whether the measurement of defecation gas is possible or not on the basis of the environment reference value. If the data analyzer 60 determines that measurement of a noise level being performed, or the measurement of defecation gas is impossible, the display device 68 is allowed to display a message, such as “During measurement preparation. Wait for a while if possible”, as shown in a lower section of FIG. 6, to urge a test subject to wait for defecation.

Next, when the seating detection sensor 36 detects that a test subject sits on a seat, the control device 22 transmits a signal of starting step S3 of setting measurement reference values to the data analyzer 60 through the transmitter-receiver 56, and then step S3 of setting measurement reference values is performed in synchronization with the data analyzer 60. If the seating detection sensor 36 repeats detection and non-detection predetermined times, this state is caused by influence of cleaning of the seat by the test subject, whereby it is desirable to return to S1 in this kind of state.

In step S3 of setting measurement reference values, the data analyzer 60 determines noise of stink gas attached to a test subject, which is noise to measurement of defecation gas of a test subject, on the basis of a measurement value measured by the odiferous gas sensor 26. Detection data of odiferous gas other than defecation gas, cause by a test subject can be utilized in diagnosis of a specific disease different from a colorectal cancer. Further, if a measurement value measured by the odiferous gas sensor 26 is abnormally large and is unstable, it is determined that there is a possibility that disinfection is performed by using alcoholic disinfectant or the like to continue the display, “During measurement preparation. Wait for a while if possible”, shown in the lower section of FIG. 6. Alternatively, if a level of noise caused by a test subject is a predetermined value or more, the data analyzer 60 transmits a signal to the nozzle driving device 42 of a local cleaning device to allow the nozzle driving device 42 to operate to clean the anus of a test subject, or the data analyzer 60 allows the display device 68 to notify a test subject that anus cleaning should be performed. On the other hand, if a measurement value measured by the odiferous gas sensor 26 is sufficiently reduced, this display is erased. In addition, if a measurement value measured by the odiferous gas sensor 26 is insufficiently reduced even if a predetermined time has elapsed, the data analyzer 60 stops measurement of physical condition and allows the display device 68 to display the stop to notify a test subject.

In addition, in step S3 of setting measurement reference values, the data analyzer 60, as described later, sets a reference value for estimating the amount of gas, on the basis of concentration of gas measured by the odiferous gas sensor 26.

Next, the data analyzer 60, as described in detail later, determines that a test subject performs an excretory act if detection data by the odiferous gas sensor 26 rises with a positive rate of change of a predetermined value or more from the reference value of the odiferous gas noise, and proceeds to step S4 of measurement. The data analyzer 60 performs step S4 of measurement from when determining that the test subject performs an excretory act until when the seating detection sensor 36 detects that the test subject leaves the seat.

That is, in a period after the entrance detection sensor 34 detects that a test subject enters the toilet installation room until the seating detection sensor 36 detects that the test subject sits on a seat, it is estimated that the test subject shuts the door of the toilet installation room, approaches the flush toilet 2 to open the lid, turns back in such a manner as to face his or her back to the flush toilet 2, and thereafter, performs a preparatory act of defecation such as undressing. Consequently, in the present embodiment, the period after a test subject enters the toilet installation room until the test subject sits on the seat 4 is set as “a defecation preparation period”, and a period after the test subject sits on the seat until the test subject leaves the seat is set as “a defecation period”.

Further, in the present embodiment, start of the “defecation preparation period” is determined on the basis of the entrance detection sensor 34. However, when the present invention is applied to a portable flush toilet which is installed in a bedroom or the like, a test subject is present in the space where the flush toilet is installed before the test subject starts preparation of defecation, and start of the “defecation preparation period” cannot be determined by entrance to the room. In such a case, start of the “defecation preparation period” can be determined by approach of the test subject to the flush toilet 2, opening of the lid of the flush toilet 2, input to the test subject identification device 62, an exclusive switch for inputting start of preparation for defecation or the like.

In step S4 of measurement, the control device 22 allows a storage device to store detection data for each test subject identified by test subject identification device 62, the detection data being measured by the hydrogen gas sensor 24, the odiferous gas sensor 26, the carbon dioxide sensor 28, the humidity sensor 30, the temperature sensor 32, the entrance detection sensor 34, the seating detection sensor 36, and the defecation/urination detection sensor 38. The control device 22 transmits these measurement values stored in the storage device to the data analyzer 60 through the transmitter-receiver 56, after step S4 of measurement is finished. In the present embodiment, although the measurement values are transmitted to the data analyzer 60 from the control device 22 after step S4 of measurement is finished, besides this, the measurement values may be transmitted in real time in parallel with measurement.

The control device 22 starts measurement of defecation gas even if a test subject inputs no information identifying the test subject into the test subject identification device 62. After then, if the test subject inputs information on the test subject during one defecation, detection data detected before the information is inputted is stored in the storage device in association with the inputted information on the test subject. This is a practical device corresponding to characteristics of defecation, in which a test subject is first allowed to perform no various kinds of input in an urgent situation of defecation, and to perform the input after calming down. In addition, if the test subject inputs no information on the test subject even if a predetermined time has elapsed after measurement has been started, the display device 68 and the speaker 70 output a message for urging the test subject to perform the input to notify the test subject. Accordingly, it is possible to prevent a test subject from omitting input.

At the same time, as with step S3 of setting measurement reference values, the data analyzer 60 determines whether measurement is possible or not. If the data analyzer 60 determines that the measurement is possible, the data analyzer 60 allows the display device 68 to display a message that the measurement being performed to the test subject, such as “Subject: Mr. Taro Toto (identification information on a test subject)”, and “Measurement is ready. Measurement being performed”, as shown in the lower section of FIG. 6.

Next, when the seating detection sensor 36 detects that a test subject leaves the seat, the control device 22 transmits a signal of starting step S5 of medical examination to the data analyzer 60 through the transmitter-receiver 56. When receiving the signal, the data analyzer 60 starts step S5 of medical examination.

The data analyzer 60 first calculates reliability of measurement that is described later, on the basis of a measurement value measured by each sensor.

On the other hand, if no information identifying a test subject is inputted after the test subject has left the seat, the control device 22 prohibits cleaning of the flush toilet 2. That is, if no information for identifying a test subject is inputted, the control device 22 does not allow the flush toilet 2 to discharge cleaning water and allows a message urging the test subject to perform input to be displayed even if the test subject operates a cleaning button (not shown) of the remote control 8. Accordingly, it is possible to strongly urge a test subject to input information for identifying a test subject.

The data analyzer 60 also estimates the amounts of odiferous gas and hydrogen gas (healthy-state gas).

In step S5 of medical examination, the data analyzer 60 performs calculation of results of a medical examination to analyze physical condition of a test subject on the basis of time-dependent change in a plurality of detection data items that is detected in defecation performed multiple times in a predetermined period and that is stored in a storage device, as well as performs time-dependent diagnosis based on stored values, and then selects advice contents based on the time-dependent diagnosis. The data analyzer 60, as shown in a third section from the top of FIG. 7, allows the display device 68 to display advice contents selected as a message related to health management. In an example shown in FIG. 7, present physical condition of a test subject that corresponds to “insufficient physical condition” is displayed as a result of a medical examination is displayed, as well as “Intestinal environment may be wrong. Make efforts to have a healthy living habit” is displayed as an advice.

In a portion below that of the result of a medical examination, there is displayed the amount of healthy-state gas, such as hydrogen gas, and carbon dioxide gas, as well as the amount of wrong physical condition state gas, such as odiferous gas, in the measurement in this time. In a portion below that of the advice, measurement results of previous four times measurements are displayed together. If a test subject presses a button of “detailed screen” in a display screen, there is displayed a table showing change in physical condition of a test subject for the last one month. This display will be described later. In this way, analysis results displayed in the display device 68 of the remote control 8 include only a state of physical condition, an advice, and change in physical condition (history of measurement data), and include no notification related to a determination result of disease of cancer, such as displayed in the medical facility terminal 16. These analysis results may be notified in the terminal 14 for a test subject.

As shown in a lowermost section of FIG. 7, reliability of measurement data in this time is displayed in a lower portion of a screen of the display device 68. In the example shown in FIG. 7, the reliability is displayed as “4” that is relatively high. If the reliability is low, a cause of decrease in reliability as well as an advice for improving the decrease is displayed in a portion below that of display of the reliability. For example, if residual gas noise caused by gas remaining in a bowl, or test subject noise caused by a test subject, is large, a test subject is notified that the noise reduces the reliability to affect measurement results.

Next, when the entrance detection sensor 34 detects that a test subject leaves the toilet installation room R, the control device 22 transmits a signal of transmitting data to the data analyzer 60 through the transmitter-receiver 56. When receiving the signal, the data analyzer 60 performs step S6 of communication.

In step S6 of communication, the data analyzer 60 transmits the following to the server 12 through a network: information for distinguishing a test subject identified by the test subject identification device 62; data measured by various sensors; calculated reliability; information on a measurement date and time; stool condition information on at least one of the amount of stool and a state of the stool acquired by the defecation/urination detection sensor 38; and notifying data including defecation history information. The server 12 records the information received in a database.

The control device 22 also performs step S7 of improving environment after measurement after the entrance detection sensor 34 has detected that a test subject has left the toilet installation room R.

The control device 22 allows the odiferous gas sensor 26 to measure concentration of gas in step S7 of improving environment after measurement. If concentration of gas measured by the odiferous gas sensor 26 is larger than a predetermined value even if a predetermined time has elapsed after a defecation period has been finished, the control device 22 determines that there is a stool attached to the bowl 2a of the flush toilet 2 to allow the toilet cleaning device 46 to discharge cleaning water stored in a cleaning water tank into the bowl 2a to clean the inside of the bowl 2a, or to allow the toilet disinfection device 48 to create disinfecting water, such as hypochlorous acid water, from tap water, or the like to spray disinfecting water created onto the bowl 2a to disinfect the bowl 2a.

Toilet cleaning which is performed automatically as the additional toilet cleaning by the toilet cleaning device 46 is set so that its cleaning capability is higher than that of usual toilet cleaning performed by allowing a test subject to operate a cleaning switch (not shown) of the remote control 8. Specifically, it is preferable that the toilet cleaning performed automatically is set to have a high frequency of discharge of cleaning water into the bowl 2a, or flow velocity of the cleaning water is set high. The disinfection of the bowl 2a performed automatically is set so that its disinfection capability is higher than that of usual disinfection of the bowl performed by allowing a test subject to operate a disinfection switch (not shown) of the remote control 8. Specifically, the disinfection of the bowl performed automatically is set so that water for disinfection of higher concentration as compared with usual disinfection is sprayed, or a large amount of water for disinfection is sprayed.

If concentration of gas measured by the odiferous gas sensor 26 is more than a predetermined value even if a predetermined time has elapsed after a defecation period has been finished, the control device 22 determines that there is a contamination in the duct 18a to allow the duct cleaner 58 to operate. The duct cleaner 58 cleans the inside of a duct 18a attached to the suction device 18 with hypochlorous acid acquired by electrolysis of tap water, or the like.

If concentration of gas measured by the odiferous gas sensor 26 does not decrease sufficiently and is still more than the predetermined value even if the cleaning and the disinfection processing, described above, are performed, the control device 22 allows the display device 68 to display a message of encouraging cleaning of the flush toilet 2.

Then, in step S7 of improving environment after measurement, the control device 22 allows the sensor heater 54, the suction device 18, and the toilet lid opening/closing device 40 to switch to the measurement waiting mode to finish one measurement.

Next, with reference to FIG. 8, the physical condition display table will be described. The physical condition display table is to be displayed by pressing the button of “detailed screen” in the display screen shown in FIG. 7. A storage device on the remote control 8 side stores the physical condition display table, defecation dates and times of a test subject in association with identification information on the test subject, and previous measurement data, for each test subject. Although the previous measurement data stored in the storage device on the remote control 8 side may be data throughout a defecation period, measurement data on defecation gas discharged by the first excretory act in the defecation period (the first measurement data during the excretory act) is preferable due to capacity of the storage device.

As shown in FIG. 8, the physical condition display table is determined on the basis of an experiment performed by the present inventors, described above, and is a graph in which the vertical axis represents an index related to the amount of odiferous gas (referred to as wrong physical condition state gas in the display), referred to as a first index, and the horizontal axis represents an index related to the amount of healthy-state gas, referred to as a second index. The first index relates to the amount of odiferous gas based on first detection data detected by the gas detector 20, and the second index relates to the amount of hydrogen gas of healthy-state gas based on second detection data detected by the gas detector 20. The display device 68 of the remote control 8 displays the physical condition display table with the vertical axis and the horizontal axis as above, in which a measurement result of defecation gas of a test subject is plotted in a time-dependent manner. That is, as shown in FIG. 8, a plotted point representing the latest measurement result of the same test subject is referred to as “1”, that representing the last result is referred to as “2”, that representing the last but one result is referred to as “3”, and the like, and then each of plotted points of the last thirty times is displayed with a numeral. Accordingly, a test subject can recognize time-dependent change in his or her own physical condition. Although the present embodiment displays plotted points of thirty times, those of a few weeks and a few months may be available, or those in units of year may be also available because cancer develops in years. It is more desirable to enable a test subject to change a display range according to a situation. Further, it is needless to say that if a display range is wide, it is more preferable to change a display method in consideration of viewability so that monthly averages of plotted points for one year, or two years, are used.

The physical condition display table sets regions of a plurality of stages corresponding to whether physical condition is good or wrong, in accordance with a relationship between the index related to healthy-state gas and the index related to odiferous gas, such as: a “disease suspicion level 2”, a “disease suspicion level 1”, an “insufficient physical condition level 2”, an “insufficient physical condition level 1”, and a “good physical condition”. As shown in FIG. 8, the “disease suspicion level 2” corresponding to the worst state of physical condition is set in a upper-left region in the physical condition display table, where the amount of odiferous gas is maximum and the amount of healthy-state gas is minimum. On the other hand, the “good physical condition” corresponding to the best state of physical condition is a lower-right region in the physical condition display table, where the amount of odiferous gas is minimum and the amount of healthy-state gas is maximum. The “disease suspicion level 1”, “insufficient physical condition level 2”, and “insufficient physical condition level 1”, showing physical condition levels between the worst and best conditions, are set in the order from the upper-left in the physical condition display table as belt-like regions rising diagonally up and to the right. This kind of physical condition display table is preset in accordance with weight, age, sex, and the like of a test subject, and displaying plotted points based on the first and second indexes in the table enables analysis based on detection data and test subject information to be performed.

As above, in the present embodiment, two indexes of the index related to the amount of odiferous gas and the index related to the amount of healthy-state gas are used, so that it is possible to evaluate physical condition of a test subject and change in physical condition thereof in more detail. For example, even in a case where the amount of healthy-state gas showing a good physical condition is large, if the amount of odiferous gas is also large, evaluation is not the level of the best physical condition (the upper-right region in the physical condition display table). Conversely, even in a case where the amount of healthy-state gas showing a good physical condition is very low, if the amount of odiferous gas is low, evaluation is not the level of the worst physical condition (the lower-left region in the physical condition display table).

For example, a boundary line between the “insufficient physical condition level 1” and the “insufficient physical condition level 2” showing a worse state than that of the level 1 is drawn rising diagonally up and to the right so that as the amount of the index related to healthy-state gas in the horizontal axis increases, the index related to the amount of odiferous gas in the vertical axis also increases, and the “insufficient physical condition level 2” showing a state where physical condition is wrong is distributed on a side of the boundary line where the index related to the amount of odiferous gas is large. The boundary line is set in this way, so that in the present embodiment, even if the amount of the index related to healthy-state gas in the horizontal axis is the same value, evaluation of physical condition varies depending on a value of the index related to the amount of odiferous gas in the vertical axis. In order to acquire the same evaluation, it is required that as a value of the amount of odiferous gas in the vertical axis increases, a value of the amount of healthy-state gas in the horizontal axis also increases.

The storage device on the remote control 8 side stores advices corresponding to the states of physical condition. Specifically, there are stored advices, such as: “Present to a hospital” corresponding to a state of physical condition, the “disease suspicion level 2”; “Recommend presenting to a hospital” corresponding to a state of physical condition, the “disease suspicion level 1”; “Concern for disease increases. Reduce stress and improve a living habit immediately” corresponding to a state of physical condition, the “insufficient physical condition level 2”; “Intestinal environment is wrong. Make an effort to have a healthy living” corresponding to a state of physical condition, the “insufficient physical condition level 1”; and “Physical condition is good” corresponding to a state of physical condition, the “good physical condition”. In the physical condition display table, plotted points showing physical condition of a test subject, as well as an advice corresponding to a region where the latest plotted point is positioned is displayed.

However, the display device 68 of the remote control 8 does not plot each of analysis results acquired by the data analyzer 60 as it is in the physical condition display table, and plots each of the analysis results at a position to which each of them is displaced after predetermined correction has been applied to each of them. It is assumed that the biological information measurement system 1 of the present embodiment detects disease, such as colorectal cancer, and this kind of disease does not steeply develop in a few days. Meanwhile, the biological information measurement system 1 of the present embodiment sucks defecation gas from the bowl 2a of the flush toilet 2 installed in the toilet installation room R to analyze the sucked gas, and it is impossible to collect all of the defecation gas. In addition, there is a possibility that various factors, such as that a test subject wears perfume, and that gas to which the odiferous gas sensor 26 is sensitive, such as odiferous gas, remains in the toilet installation room R, may cause an error in measurement results of physical condition.

Thus, if physical condition displayed on the basis of one measurement result of a test subject greatly inclines toward wrong physical condition, an unnecessary mental burden is applied to a test subject. In addition, if a measurement result of physical condition greatly varies for each measurement, it results in losing confidence of a test subject in a measurement result of physical condition. Thus, the biological information measurement system 1 of the present embodiment allows the data analyzer 60 to apply correction to an analysis result to prevent a measurement result to be displayed from greatly varying for each measurement. However, detection data stored in the storage device of the remote control 8 and detection data transmitted to the server 12 to be stored, to which no correction is applied, are stored along with reliability of the detection data. It is preferable that the storage device of the remote control 8 stores a coordinate of a display after correction in consideration of a next display. All of detection data acquired by the biological information measurement system 1 of the present embodiment in this way does not have high reliability. However, if data on daily defecation act is continuously acquired for a long period to be accumulated in the storage device of the remote control 8 and the server 12, it is possible to detect change in physical condition of a test subject for a long period. As a result, it is possible to call attention to a test subject before physical condition of the test subject is greatly deteriorated, to prevent the test subject from having a serious disease, such as colorectal cancer.

In the present embodiment, it is not always required to apply correction to detection data to be stored in the storage device of the remote control 8, and also detection data after the correction may be stored.

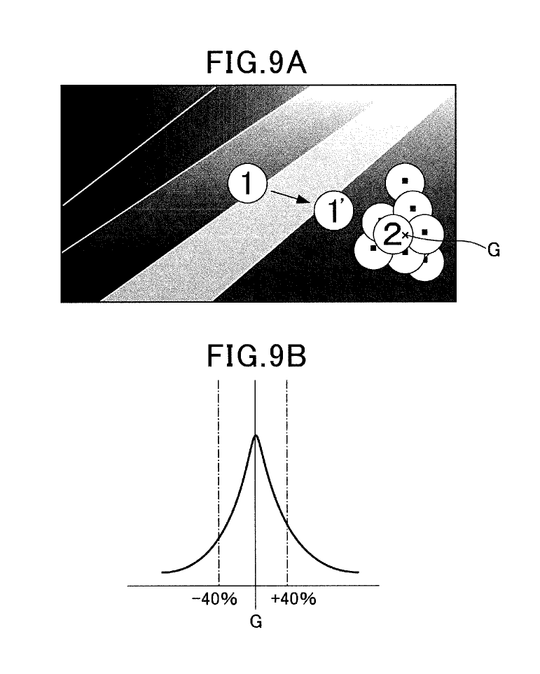

Next, with reference to FIG. 9, correction of plotted points will be described.

FIG. 9A shows an example of displacement of a plotted point of updated data by correction, and FIG. 9B shows limit processing with respect to the amount of displacement of a plotted point.

First, as shown in FIG. 9A, a plotted point calculated by the data analyzer 60 on the basis of the latest measurement is represented as “1”, and the point is greatly displaced from the center G of an area of plotted points of measurement data of the last thirty times. In this way, if the plotted point “1” that is greatly displaced from distribution of measurement data up to the previous measurement is displayed, an excessive mental burden may be applied to a test subject. Since a risk of cancer does not increase in a day, it is highly possible that this kind of large change in measurement data does not show an increase in a risk of cancer, but a result of a bad living habit in the previous day, or influence of noise. In the present embodiment, correction is performed in a manner that gives due consideration for applying no excessive mental burden to a test subject. Thus, if the latest analysis result varies toward a wrong physical condition side (in an upper-left direction), the data analyzer 60 displaces a position at which the plotted point “1” is displayed in the physical condition display table toward the center G of an area by a predetermined distance on the basis of reliability of measurement data in this time to allow the plotted point “1” to be displayed. That is, in an example shown in FIG. 9A, the latest measurement data is displayed at a position of a plotted point “1′” acquired by correcting the plotted point “1” so that the plotted point “1” is displaced toward the center G of an area (on a good physical condition side), and the plotted point “1” is not actually displayed. A displacement distance of the plotted point “1” toward the center G of an area direction increases, as reliability of the latest measurement data decreases. In this way, displacing the latest plotted point on a side showing good physical condition enables a mental burden to a test subject to be reduced. However, if displacement of the latest plotted point toward the wrong physical condition side continues predetermined times or more, the data analyzer 60 reduce the amount of correction (the amount of correction of displacement). Accordingly, a test subject can recognize that his or her own physical condition is deteriorated, and can be encouraged to make an effort to improve the physical condition.

If a very large noise is applied to the latest measurement of physical condition to very greatly shift the latest plotted point, it is thought that physical condition displayed may be greatly displaced toward the wrong physical condition side even if the correction described in FIG. 9A is applied. Thus, as shown in FIG. 9B, there is a predetermined limit of a displacement distance of the latest data from the center G of an area. That is, displacement of the latest data from the center G of an area is limited to a range of ±40% of a coordinate value of the center G, and even if the latest data is displaced by 40% or more from the coordinate of the center G of an area, the latest data is plotted at a position displaced by 40%. For example, in a case where a coordinate value of the center G of an area is represented as (x, y), a range of coordinate values at which the latest data can be plotted is represented as (0.6x to 1.4x, 0.6y to 1.4y), and the latest data is not plotted at a position out of the range.

In addition, if displacement of the latest data exceeding this kind of 40% continues twice, a range in which the latest data can be displaced is eased to 60%. Accordingly, for example, if the coordinate value of the center G of an area is represented as (x, y), a range of coordinate values at which the latest data can be plotted is changed to that represented as (0.4x to 1.6x, 0.4y to 1.6y). Because it is thought that if a large displacement of the latest data as above occurs at high frequency, it is not a mere measurement error, but a reflection of some sort of change in physical condition of a test subject.

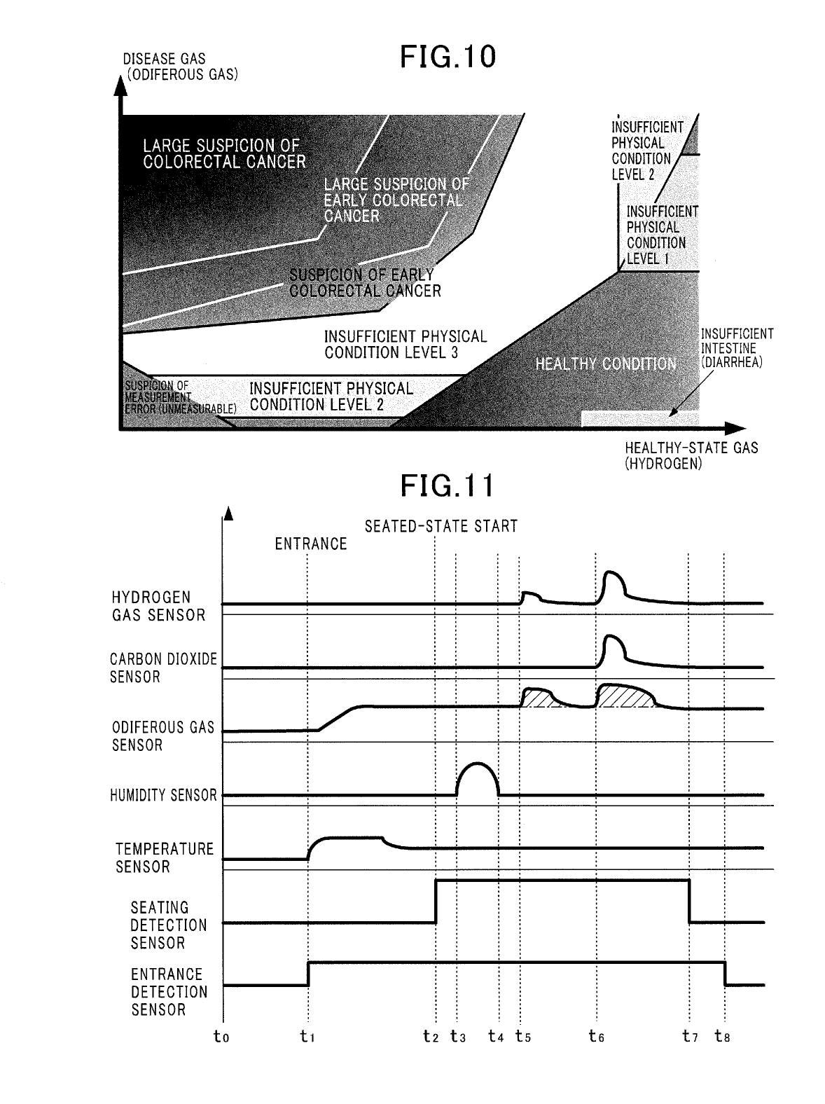

Next, with reference to FIG. 10, a diagnosis table on a server side will be described. Processing in the server below is performed by a server side data analyzer, provided in the server 12.

FIG. 10 shows an example of a diagnosis table displayed on the server side. As described above, in the biological information measurement system 1 of the present embodiment, measurement data for all defecation periods analyzed by the data analyzer 60 is sequentially transmitted to the server 12 through the Internet to be stored in a database on the server side. This accumulated measurement data can be displayed in the medical facility terminal 16 installed in a medical facility 302 registered by a test subject. For example, when a test subject has a medical examination in the medical facility after receiving the message, “Recommend presenting to a hospital” displayed in the display device 68 of the remote control 8, the medical facility terminal 16 enables a diagnosis table for a server to be displayed. In the diagnosis table, its vertical axis and horizontal axis represent the same indexes as those of the physical condition display table to be displayed in the display device 68 of the remote control 8, and a state of physical condition assigned to each region is more specific. A doctor refers to measurement data on a test subject stored in a database on a server 12 side in the medical facility terminal 16 to be able to refer to time-dependent physical condition of the test subject, and thus the data can be useful for inspection and treatment in the medical facility. Alternatively, it is also possible to configure the present invention so that if measurement data transmitted to the server 12 shows excessive wrong physical condition, a medical facilities registered by a test subject notifies the terminal 14 for a test subject, corresponding the test subject, of encouraging the test subject to have a medical examination.

The diagnosis table displayed in the medical facility terminal 16 is different from the physical condition display table displayed in the display device 68 of a test subject as described above. As shown in FIG. 10, the diagnosis table on the server 12 side is determined on the basis of an experiment performed by the present inventors, and in the diagnosis table, a disease state is associated corresponding to a relationship between the amount of healthy-state gas and the amount of odiferous gas. Specifically, in the diagnosis table, the following regions are set corresponding to a relationship between the amount of healthy-state gas and the amount of odiferous gas: “Large suspicion of colorectal cancer”, “Large suspicion of early colorectal cancer”, “Suspicion of early colorectal cancer”, “Insufficient physical condition level 3”, “Insufficient physical condition level 2”, “Insufficient physical condition level 1”, “Healthy condition”, “Insufficient intestine (diarrhea)”, and “Suspicion of measurement error”.

In a diagnosis table on the server side, set in this way, previous measurement data on a test subject is plotted in a time-dependent manner on the basis of a position of a plotted point to perform determination of disease of cancer, such as: “Large suspicion of colorectal cancer”, “Large suspicion of early colorectal cancer”, and “Suspicion of early colorectal cancer”. No correction as well as no limit is applied to a plotted point displayed in the diagnosis table on the server side, so that a doctor checks data displayed for diagnosis along with its reliability in a comprehensive manner. Since a diagnosis table and a determination result displayed in the medical facility terminal 16 are set based on the premise that a doctor refers to them, a name of disease, development thereof, and the like, are more specifically displayed. If plotted points are positioned, for example, in regions related disease of cancer, such as the “Large suspicion of colorectal cancer”, “Large suspicion of early colorectal cancer”, and “Suspicion of early colorectal cancer”, for a long time, a message of a high possibility of disease is displayed. A doctor is able to check plotted points shown, reliability of measurement, and the like, for diagnosis in a comprehensive manner to notify a test subject of a state of the physical condition. The medical facility terminal 16 is configured to be capable of also displaying reliability calculated by referring to a database, data measured by various sensors, information on stool condition related to at least one of the amount of stool and condition of stool, and defecation history information, along with a diagnosis table in which previous measurement data is plotted in a time-dependent manner.

A large number of devices 10 on a test subject side are connected to the server 12, a large number of measurement data items of test subjects are accumulated in the server 12. In addition, a database on the server 12 side also accumulates data on disease condition acquired from a result of detailed examination of a test subject, performed in a medical facility, after the test subject has had a medical examination in the medical facility on the basis of certain measurement data. Thus, it is possible to accumulate data acquired by associating data measured by the biological information measurement system 1 of the present embodiment with actual disease condition, on the server 12 side. The diagnosis table on the server side is sequentially updated on the basis of measurement data on a large number of test subjects accumulated in this way, so that it is possible to perform diagnosis with higher accuracy on the basis of the updated diagnosis table.

It is also possible to update the physical condition display table on the basis of the data accumulated on the server side. The physical condition display table updated on the basis of the data on the server side is downloaded into each of the devices 10 on a test subject side through the Internet to be displayed in the display device 68 of the remote control 8. Even if the physical condition display table is updated, a message to be shown to a test subject is corrected to an appropriate content in the physical condition display table that is to be directly presented to the test subject. The present invention can be also configured to update the physical condition display table in the device 10 on a test subject side of the test subject so that an examination result is reflected, when the test subject consults a medical facility after the message “Recommend presenting to a hospital” is displayed in the display device 68, and undergoes a thorough examination, which shows that the test subject is in such a state of health that the test subject should not have concern about a disease. Accordingly, the message encouraging to present to a hospital can be prevented from being repeatedly displayed to a test subject who is determined as healthy, and applying an unnecessary mental burden to the test subject.

Next, with reference to FIG. 11, data detected by each of sensors provided in the biological information measurement system 1 of the present embodiment, and estimation of the amount of gas based on the data, will be described.

FIG. 11 is a graph schematically showing a detection signal of each of the sensors provided in the biological information measurement system 1 in one excretory act of a test subject. FIG. 11 shows a waveform of a detection signal of each of the sensors, such as the hydrogen gas sensor 24, the carbon dioxide sensor 28, the odiferous gas sensor 26, the humidity sensor 30, the temperature sensor 32, the seating detection sensor 36, and the entrance detection sensor 34, in the order from an upper section.

Estimation of the amount of gas based on a detection signal of each of the sensors is performed by the data analyzer 60 serving as physical condition state discrimination means for discriminating a physical condition state, that is, by a CPU built in the remote control 8 and a storage device, or by a CPU of the server 12 and a storage device. In the data analyzer 60, there are preset a starting threshold value of a rate of change in the amount of gas for determining starting time of an excretory act, read out from storage means of the remote control 8, and a stability threshold value with respect to the amount of gas, capable of allowing stable measurement to be performed. The term, an excretory act, here includes a fart.

First, at time t1 of FIG. 11, the entrance detection sensor 34 detects entrance of the test subject. The data analyzer 60 allows the odiferous gas sensor 26 to measure the amount of odiferous gas even in a state before the entrance detection sensor 34 detects entrance of the test subject into the toilet installation room R (time to to t₁). Even in this case, the odiferous gas sensor 26 reacts due to influence of aromatic, and remaining stool attached to the bowl 2a of the flush toilet 2 to output a certain level of a detection signal. In this way, a measurement value of the odiferous gas sensor 26 before entrance of the test subject is set as an environment reference value of the amount of gas that is residual gas noise. In a state before the entrance detection sensor 34 detects entrance of the test subject, the odiferous gas sensor 26 and the suction device 18 are in a power saving state. Accordingly, temperature of the sensor heater 54 for heating a detecting portion of the odiferous gas sensor 26 is set lower, and a rotation speed of the suction fan 18c is also reduced to reduce a flow rate of passing air.

When the entrance detection sensor 34 detects entrance of the test subject at the time t₁, the odiferous gas sensor 26 and the suction device 18 are in a startup state. Accordingly, temperature of the sensor heater 54 of the odiferous gas sensor 26 increases, as well as a rotation speed of the fan of the suction device 18 increases to suck gas at a predetermined flow rate. As a result, a detection value by the temperature sensor 32 temporarily greatly increases, and then converges to a proper temperature (after the time t₁ of FIG. 11). That is, if the entrance detection sensor 34 determines entrance of the test subject, the control device 22 determines that the “defecation preparation period” by the test subject is started, and allows the temperature of the detecting portion of the odiferous gas sensor 26 to rise to the first temperature which is a proper temperature for measurement from the second temperature for waiting. In the present specification, a period in which the entrance detection sensor 34 detects entrance of the test subject into the toilet installation room R (time t₁ to t₈ of FIG. 11) is referred to as one “defecation act”. When the test subject enters the toilet installation room R, a detection signal detected by the odiferous gas sensor 26 increases, because the odiferous gas sensor 26 reacts to a body odor of the test subject, perfume and hair liquid used by the test subject, and the like. That is, an increment from residual gas noise before the test subject enters the toilet installation room R is test subject noise caused by the test subject. A noise measurement circuit built in the data analyzer detects residual gas noise caused by gas remaining in the bowl 2a, and test subject noise caused by the test subject. The odiferous gas sensor 26 is set at a very high sensitivity to detect a very trace amount of odiferous gas contained in the order of ppb in defecation gas discharged into a toilet to react even to the order of odor to which a human's sense of smell is insensitive.

As above, when the test subject enters the toilet installation room, the detection signal by the odiferous gas sensor 26 rises. However, the data analyzer 60 does not adopt the rise in the detection signal as the first detection data for use in analysis of the physical condition of the test subject, during the “defecation preparation period”. That is, it is highly possible that the rise in the detection signal of the odiferous gas sensor 26 before the test subject sits on the seat 4 is caused by a body odor or a perfume of the test subject, or disinfection by alcohol for the seat 4.

Further, it is conceivable that the detection signal by the odiferous gas sensor 26 in the defecation preparation period is the result of reacting to odiferous noise by odiferous gas remaining in the toilet installation room and odiferous gas attached to the test subject. In the present embodiment, the data analyzer 60 sets a noise level (a detection signal at a time t₂ in FIG. 11) of odiferous noise in a final stage of the “defecation preparation period” as a reference value of odiferous noise. The final stage of the “defecation preparation period” is preferable for setting the reference value of odiferous noise, because in the final stage, the test subject has been sufficiently close to the flush toilet 2, and has finished undressing, and accordingly the noise level is stable. The period after the test subject sits on the seat 4 until the test subject starts an excretion is preferable for setting the reference value of odiferous noise, because an opening portion of the flush toilet 2 is covered with a body of the test subject, and accordingly a state of the gas in the bowl 2a is stable. In this way, it is preferable that the reference value of the odiferous noise is set by the noise levels before and after transition from the “defecation preparation period” to the “defecation period”, that is, before and after the test subject sits on the seat.

Next, when the seating detection sensor 36 detects that the test subject sits on the seat 4 at time t₂ of FIG. 11, this time point is set as a starting point of one defecation period of the test subject. In the present specification, a period in which the seating detection sensor 36 detects whether the test subject sits on the seat 4 (time t₂ to t₇ of FIG. 11) is referred to as one “defecation period”.

In an example shown in FIG. 11, a detection value of the humidity sensor 30 increases in a period between the time t₃ and the time t₄ after the test subject has sat on the seat 4 at the time t₂, because urination of the test subject is detected. Then, since there is little change in a detection value of odiferous gas sensor 26, the data analyzer 60 determines that an excretory act is not performed. In this way, urination by a test subject hardly influences the detection value of the odiferous gas sensor 26, because discharged urea immediately flows into standing water in the bowl 2a. Subsequently, a detection value of each of the hydrogen gas sensor 24 and the odiferous gas sensor 26 steeply rises at the time t₅. In this way, if the detection value of the odiferous gas sensor 26 steeply rises with a positive rate of change of a predetermined value or more from the reference value of the odiferous noise in a defecation period after the test subject has sat on the seat 4, the data analyzer 60 determines that an excretory act is performed.

If a steep rise of the detection data like this is detected, the data analyzer 60 starts to acquire the detection data for measuring physical condition. That is, since it is highly possible that a steep rise of the detection data by the odiferous gas sensor 26 in the “defecation period” is caused by an excretory act by a test subject, if a rise like this is detected, the data analyzer 60 adopts the detection data after the rise in analysis of physical condition of the test subject as first detection data.

When the excretory act is performed, the data analyzer 60 estimates the amount of odiferous gas discharged from the test subject on the basis of a fluctuation range of an increment of a detection value of the odiferous gas sensor 26 from the reference value of residual gas (a hatched area in a graph of detection values of the odiferous gas sensor 26). That is, the data analyzer 60 sets a value of detection data at the starting point of the defecation period of the test subject as the reference value of odiferous noise which is a noise level caused by the test subject to estimate the amount of odiferous gas by the first excretory act on the basis of a difference between the detection value detected by the odiferous gas sensor and the reference value. In this way, since the data analyzer 60 estimates the amount of odiferous gas on the basis of a difference from a reference value, it is possible to reduce influence of noise caused by a test subject. If a noise level caused by the test subject is a predetermined value or more, the data analyzer 60 allows the display device 68 to notify the fact. Likewise, the data analyzer 60 estimates the amount of hydrogen gas discharged from the test subject on the basis of an increment of a detection value of the hydrogen gas sensor 24 from a reference value of residual gas. After an excretory act of the test subject has been performed (after the time t₅ of FIG. 11), a detection value of each of the odiferous gas sensor 26 and the hydrogen gas sensor 24 returns to the reference value of residual gas. Subsequently, when the second excretory act of the test subject is performed at the time t₆, a detection value of each of the odiferous gas sensor 26, the carbon dioxide sensor 28 and the hydrogen gas sensor 24 steeply rises again. For the second excretory act, as with the first excretory act, the amount of odiferous gas and the amount of hydrogen gas, discharged from the test subject, are also estimated on the basis of an increment from the reference value of residual gas. When the amount of odiferous gas and the amount of hydrogen gas of the second excretory act or later are estimated, the reference value may be changed for each excretory act in consideration of influence of floating stool in seal water in the bowl, and the like.

Subsequently, the seating detection sensor 36 detects that the test subject leaves the seat at the time t₇ of FIG. 11 to finish the one defecation period, and then the entrance detection sensor 34 detects that the test subject leaves the toilet installation room at the time t₈ to finish the one defecation act. The data analyzer 60 estimates the amount of defecation gas by excretory act of each time until the entrance detection sensor 34 detects that the test subject leaves the toilet installation room.

Each of the remote control 8 and the server 12 determines physical condition of the test subject on the basis of the amount of defecation gas measured in this way. In this case, it is desirable to enable measurements of physical condition to be displayed on the remote control 8 side during a defecation period, or immediately after the defecation period has been finished. Then, if excretory acts are performed multiple times, stools accumulate in the bowl 2a to reduce accuracy of measurement of the amount of defecation gas, based on odiferous gas. Meanwhile, in the first excretory act, defecation gas reaching the most downstream portion of the large intestine is discharged, so that it is possible to acquire most useful information for measurement of physical condition to increase reliability of the measurement. Based on the fact, on the remote control 8 side, when the amount of defecation gas (the amount of odiferous gas and hydrogen gas) by the first excretory act is estimated, physical condition of a test subject is measured on the basis of only the amount of defecation gas by the first excretory act to be displayed in the display device 68 of the remote control 8. Alternatively, it is also possible to measure a state of physical condition by allowing a weighting of a measurement value based on detection data on an initial excretory act in one defecation act to be higher than a weighting for a later excretory act.

In the physical condition display table displayed in the display device 68, a vertical axis represents a concentration of odiferous gas based on the first detection data, a horizontal axis represents a concentration of hydrogen gas based on the second detection data, and a physical condition state of a test subject is displayed as a plotted point in the table. Here, as described by FIG. 11, the first and second detection data are acquired for each excretory act during one defecation period (for example, after the time t₅, after the time t₆ in FIG. 11). The physical condition of the test subject is measured by a correlation between the odiferous gas and healthy-state gas contained in defecation gas, and therefore a plotted point displayed in the physical condition display table needs to be based on the first and second detection data concerning the same excretory act. It is preferable that the detection data adopted in analysis of physical condition relates to an excretory act at an early stage in one defecation period.

In contrast, on the server 12 side, it is desirable to accurately perform determination by using a total amount of defecation gas by excretory acts of multiple times. Thus, on the server 12 side, a state of physical condition of a test subject is determined on the basis of a total amount of defecation gas by excretory acts of multiple times (a total amount of odiferous gas and hydrogen gas), or more preferably, on the basis of a total amount of defecation gas by every excretory act included in one defecation period from sitting on a seat to leaving the seat. Although determination of a state of physical condition of a test subject on the server 12 side does not always require a total amount of defecation gas by every excretory act included in one defecation period, it is preferable that the determination is based on a total amount of defecation gas by every excretory act included in defecation periods of multiple times.

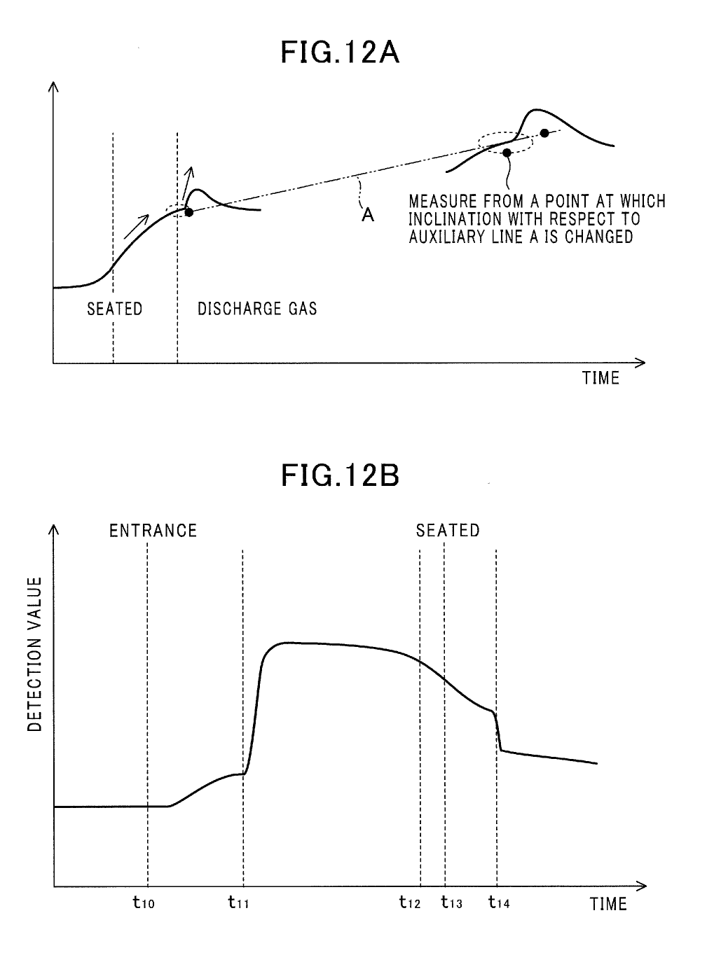

In the example shown in FIG. 11, although the reference value of residual gas is constant, it is possible to estimate the amount of discharge of odiferous gas even if the reference value is not constant. For example, if a detection value detected by the odiferous gas sensor 26 tends to increase, as shown in FIG. 12A, a reference value is indicated as an auxiliary line A that is drawn on the assumption that a rate of change in an increase of a detection value detected by the odiferous gas sensor 26 before an excretory act is started continues before and after the excretory act. Accordingly, it is possible to estimate the amount of odiferous gas by determining that one excretory act is started at the time when an inclination of detection values of the odiferous gas sensor 26 from the auxiliary line A greatly varies.

The amount of odiferous gas is estimated on the basis of a difference from a reference value that is set by using the amount of residual gas before an excretory act, so that it is desirable that there is no large change in the reference value. Thus, if a rate of change of detection values detected by the odiferous gas sensor 26 before a starting point of an excretory act (or a rate of change of a reference value of an inclination of the auxiliary line A) is a first stability threshold value or less, the data analyzer 60 allows notification means (circuit) composed of the display device 68 of the remote control 8 or the speaker 70 to notify the fact that estimation of the amount of defecation gas has high accuracy.

Meanwhile, if a spray aromatic is sprayed immediately before an excretory act, or a disinfecting sheet of an alcoholic toilet seat disinfectant or a disinfect spray is used, a detection value detected by the odiferous gas sensor 26 before the excretory act greatly varies. If a value in this kind of state is set as a reference value, it is impossible to estimate an accurate amount of odiferous gas. Thus, if a reference value of a noise level caused by a test subject is a predetermined value or more, or a rate of change of the reference value is a predetermined rate of change or more, the data analyzer 60 allows the notification means composed of the display device 68 of the remote control 8 or the speaker 70 to notify the fact that estimation of the amount of defecation gas has low accuracy. If an excretory act is performed even if this kind of notification is performed, no measurement for analysis of physical condition is performed, or reliability of measurement is reduced.

Next, with reference to FIG. 12B, detection of use of an alcoholic toilet seat disinfectant will be described. FIG. 12B is a graph showing an example of detection values of the odiferous gas sensor 26 in a case where a test subject uses an alcoholic toilet seat disinfectant.

First, after the entrance detection sensor 34 has detected entrance of a test subject at time t₁₀ of FIG. 12B, a detection value of the odiferous gas sensor 26 gradually rises because the odiferous gas sensor 26 reacts to a body odor and the like of the test subject. Next, when the test subject takes out a seat disinfecting sheet using alcoholic disinfectant at time t₁₁, the odiferous gas sensor 26 reacts to a smell of alcohol so that its detection value steeply rises. When the test subject finishes disinfecting the seat 4 at time t₁₂, and throws away the disinfecting sheet into the bowl 2a, a detection value of the odiferous gas sensor 26 immediately starts to decrease because alcoholic has high volatility. The present inventors find out that the detection value steeply increased due to the alcoholic disinfectant decreases by waiting for a while to enable measurement because characteristics of the alcoholic disinfectant described above is different from those of remaining stink gas components. However, in a case of disinfect with an alcoholic disinfecting sheet, the sheet may float in seal water when thrown away. In this case, the alcohol continues to vaporize so that the decrease of the detection value steeply increased tends to be delayed. Thus, it is desirable to discharge the sheet as described below.

Subsequently, after the seating detection sensor 36 has detected that a test subject has sat on the seat at time t₁₃, if the test subject operates the cleaning switch (not shown) of the remote control 8 to perform cleaning of the flush toilet 2, a disinfecting sheet floating in seal water in the bowl 2a is discharged to allow a detection value of the odiferous gas sensor 26 to steeply decrease. If an alcoholic disinfectant is used, the odiferous gas sensor 26 generally operates as above.

If a detection value of the odiferous gas sensor 26 steeply increases to a predetermined value or more, in a period after the entrance detection sensor 34 has detected entrance of a test subject, and before the seating detection sensor 36 detects that the test subject sits on the seat, the data analyzer 60 determines that the test subject disinfects the seat 4, or the like, by using an alcoholic disinfectant. The present inventors find out that it is possible to detect an act of disinfecting the seat 4 of a specific act performed by a test subject in the toilet installation room R from a detection signal of each of the entrance detection sensor 34, the seating detection sensor 36, and the odiferous gas sensor 26.

If no cleaning of the flush toilet 2 is performed for a predetermined time after use of an alcoholic disinfectant has been detected and a test subject has sat on the seat, the data analyzer 60 transmits a signal to the toilet cleaning device 46 to automatically perform toilet cleaning. In addition, if use of an alcoholic disinfectant has been detected, the disinfect noise reduction circuit allows the suction fan 18c to increase its rotation speed. Accordingly, the amount of gas sucked by the suction device 18 increases to allow alcohol components volatilized while the seat is disinfected to be actively deodorized by the deodorant filter 78, thereby enabling a detection value of the odiferous gas sensor 26 to be reduced.

In a state where use of an alcoholic disinfectant is detected, and a detection value of the odiferous gas sensor 26 increases, measurement of physical condition is stopped, and the display device 68 is allowed to display a message of waiting for defecation to notify a test subject of the message. The display device 68 is allowed to display a message of waiting for defecation until the measurement of physical condition becomes possible, to notify the test subject of the message. Accordingly, influence of noise caused by the alcoholic disinfectant is reduced. Meanwhile, a detection value of the odiferous gas sensor 26, which steeply increases by use of the alcoholic disinfectant, starts decreasing when the test subject finishes disinfection.

If a noise level detected by the odiferous gas sensor 26 is reversed to a downward tendency, the message of waiting for defecation displayed in the display device 68 is deleted to notify the fact that the measurement becomes possible. That is, in a state where a noise level caused by an alcoholic disinfectant is in a downward tendency, it is possible to detect a rising edge of a detection value of the odiferous gas sensor 26, in the downward tendency. The data analyzer 60 detects a time point when a detection value of the odiferous gas sensor 26 in the downward tendency rises, as discharge of defecation gas by a test subject. In a state where the noise level detected by the odiferous gas sensor 26 decreases at a predetermined rate of change or more, the measurement of physical condition is stopped and display of the message of waiting for defecation is continued. This is because in a state where the noise level steeply decreases, a rise of a detection value by discharge of defecation gas is masked so that it is impossible to accurately detect discharge of defecation gas. In addition, it is desirable to stop the measurement in a state where a reference value greatly decreases, because a calculation error also may increase.

If a noise level is a predetermined value or more due to use of an alcoholic disinfectant, measurement of physical condition is stopped, or reliability of measurement is reduced. As described above, if the reliability of measurement is reduced, a plotted point in the physical condition display table described in FIG. 9A is corrected to be more greatly displaced toward a region showing good physical condition. That is, if disinfection for the seat is detected, the disinfect noise-responding circuit corrects determination of physical condition to be outputted by the display device 68 toward the region showing good physical condition.

Meanwhile, if many stools are attached to the flush toilet 2, or a large amount of aromatics are used, an absolute value of the amount of gas detected by the odiferous gas sensor 26 increases, so that a detection value of the sensor may be saturated in some cases, or measurement accuracy may be out of a high measurement accuracy band. In this kind of state, it is difficult to accurately estimate a trace amount of odiferous gas. Thus, the data analyzer 60 performs no measurement of physical condition, or reduces reliability of measurement also in a case where an absolute amount of a reference value is a third stability threshold value or more.

In the database of the server 12, as described above, measurement data on the amount of odiferous gas and the amount of healthy-state gas of an additional test subject is sequentially accumulated. In addition, in the database of the server 12, a medical examination result for cancer acquired when a test subject has a medical examination at a medical facility is stored from the medical facility terminal 16 by being associated with identification information on the test subject. The server 12 updates a stored diagnosis table on the basis of this kind of medical examination result for cancer, and change in history of change in the amount of odiferous gas and healthy-state gas.

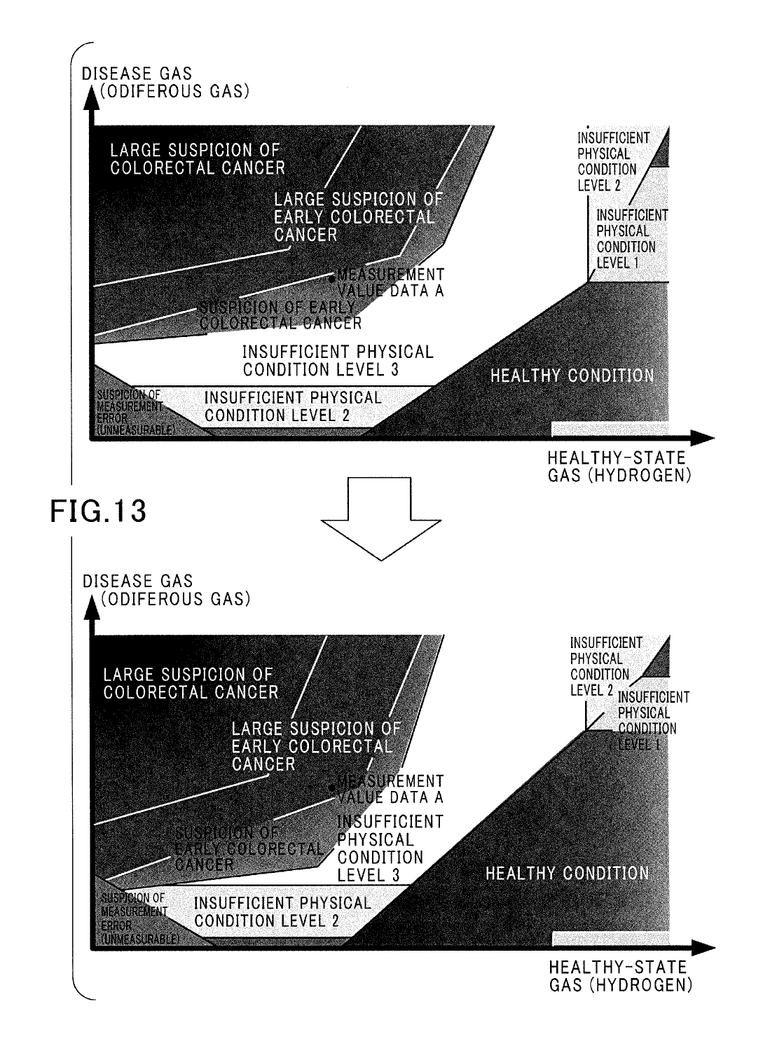

FIG. 13 shows an example of update of the diagnosis table. For example, it is assumed that analysis performed by plotting measurement data A on odiferous gas and healthy-state gas of a test subject in an old diagnosis table results in determination of the “suspicion of early colorectal cancer” is determined, and the test subject is diagnosed as early colorectal cancer by medical examination. In this kind of case, as shown in FIG. 13, the respective regions, “large suspicion of colorectal cancer”, “large suspicion of early colorectal cancer”, and “suspicion of early colorectal cancer”, are enlarged so as to include a portion corresponding to the measurement data A on the test subject diagnosed as early colorectal cancer, and the region, “insufficient physical condition level” is narrowed. Conversely, for example, in a case where there are many test subjects diagnosed as no suspicion of cancer by results of medical examination even if it is determined that the test subjects are in the region, “suspicion of early colorectal cancer” in an old diagnosis table from a correlation between the amount of odiferous gas and that of healthy-state gas, the region, “insufficient physical condition level” is enlarged, and the respective regions, “large suspicion of colorectal cancer”, “large suspicion of early colorectal cancer”, and “suspicion of early colorectal cancer” are narrowed. If the diagnosis table is updated, each of the regions in the display table is also changed.

The server 12 also stores attribute information on a test subject, such as weight, age, and sex, and a plurality of physical condition display tables classified according to a tendency of history of change in measurement data on odiferous gas and healthy-state gas.

If more detailed analysis of physical condition is requested in the device 10 on a test subject side, identification information on a test subject as well as attribute information on the test subject, such as weight, age, and sex, is registered in the server 12. When measurement data on a test subject requesting such detailed analysis is accumulated in the server 12, the server 12 selects a physical condition display table of conditions close to attribute information on the test subject, and history of change in measurement data. The server 12 then transmits the selected physical condition display table to the device 10 on a test subject side through a network. When receiving an additional physical condition display table from the server 12, the device 10 on a test subject side changes a physical condition display table that is already stored to the received physical condition display table. Accordingly, it is possible to perform accurate analysis of physical condition in accordance with the attribute of the test subject and the history of measurement data in the device 10 on a test subject side.

Although the embodiment described above is configured to store history of measurement data also in the device 10 on a test subject side, besides this, the measurement data may be stored in only the database of the server 12 so that the device 10 on a test subject side reads out history of previous measurement data from the database of the server 12 to perform calculation of results of medical examination and time-dependent diagnosis in step S5 of medical examination.

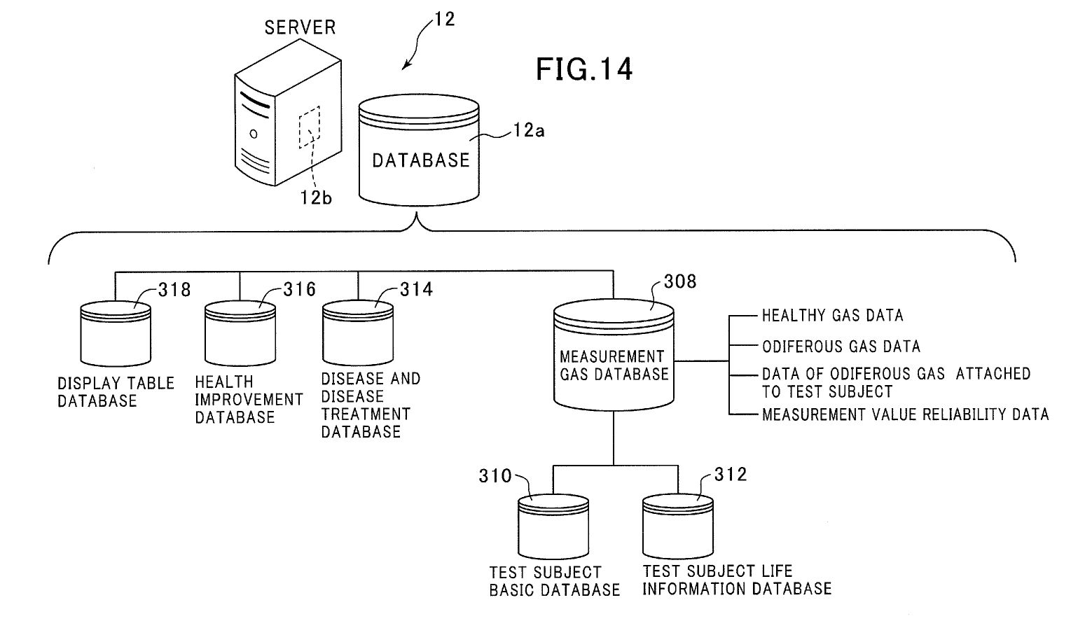

Next, with reference to FIG. 14 to FIG. 17, details of a configuration of the server 12 and data processing in the server 12 will be described.

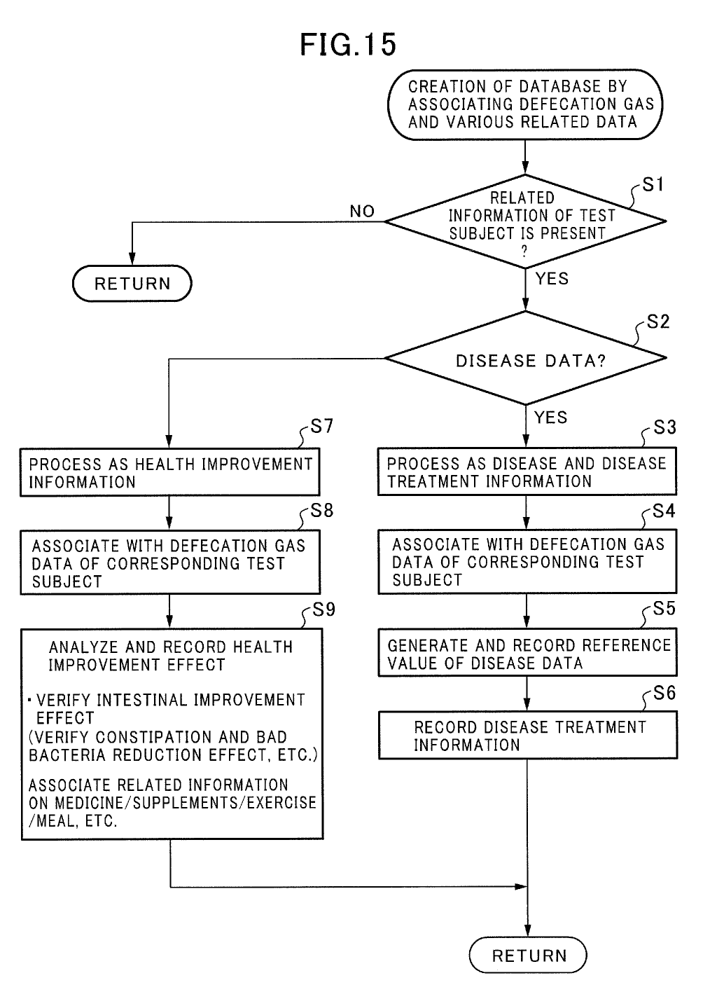

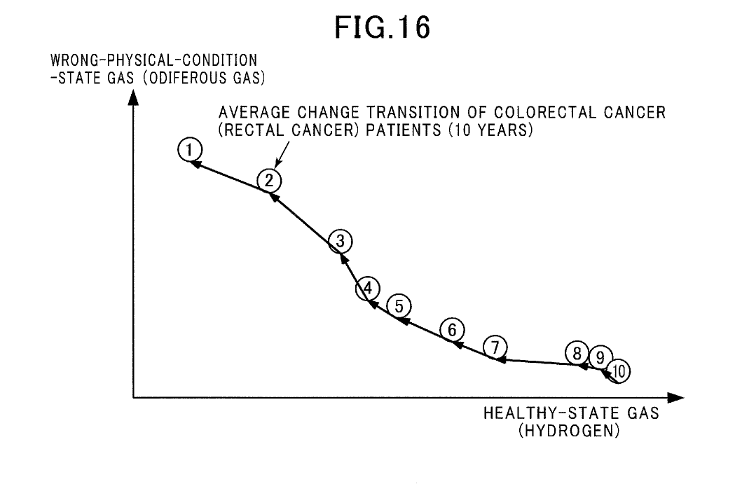

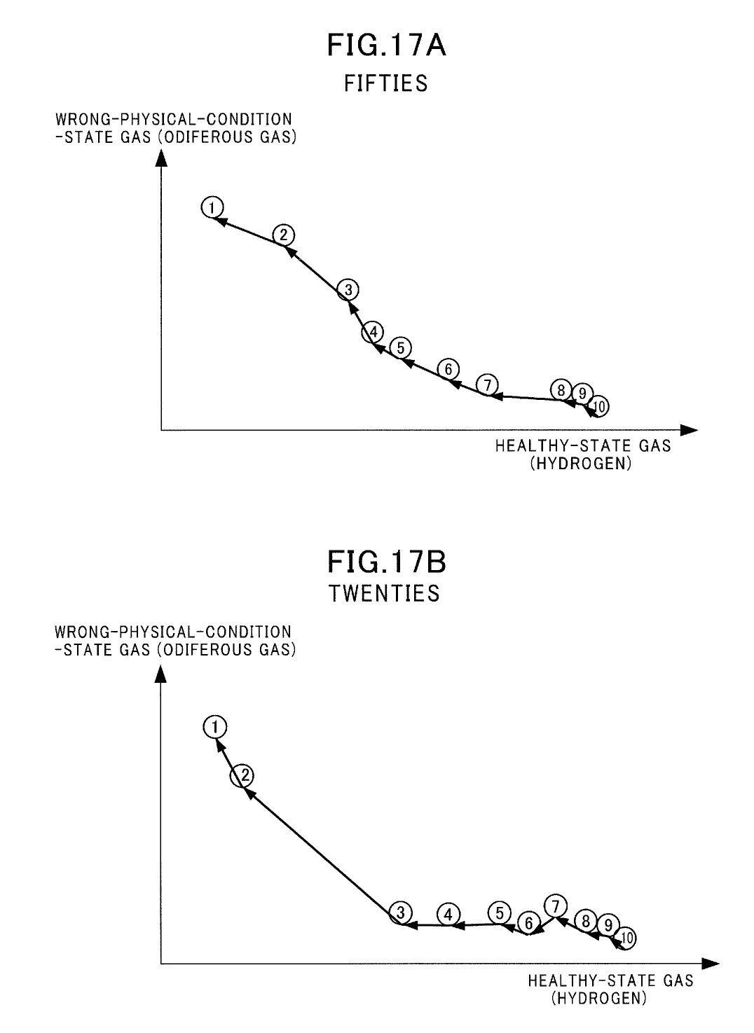

FIG. 14 shows a configuration of a database provided in the server. FIG. 15 is a flowchart showing a procedure of construction of the database. FIG. 16 shows an example of detection data of defecation gas associated with information on a disease. FIG. 17 is an example of a reference affected test subject defecation gas data obtained by totalizing detection data of defecation gas associated with information on a disease.

As shown in FIG. 14, a database 12a is provided in the server 12. The database 12a includes a measurement gas database 308, a test subject basic database 310, a test subject life information database 312, a disease and disease treatment database 314, a health improvement database 316, and a display table database 318.

Detection data of defecation gas which are acquired in the respective devices 10 on a test subject side, which perform transmission and reception with the server 12, and are transmitted are recorded in the measurement gas database 308. More specifically, in the measurement gas database 308, test subject identification information inputted in the device 10 on a test subject side, detection data of odiferous gas, detection data of healthy-state gas (hydrogen gas), and detection data of odiferous gas attached to a test subject, which are acquired, and information such as times and dates when these data were measured, reliability of the measurement data and the like are recorded. In this way, “test subject defecation gas data” including the detection data of odiferous gas, the test subject identification information, and the times and dates of excretory acts is recorded in the measurement gas database 308. The “odiferous gas attached to a test subject” refers to detection data of odiferous gas which is measured until the test subject sits on the seat after the test subject enters the toilet installation room. The detection data of odiferous gas is a noise from the viewpoint of measurement of defecation gas, but includes information on a body odor of a test subject, and is found to be useful in finding and diagnosis of other diseases different from colorectal cancer by the present inventor, as will be described later.

Meanwhile, in the test subject basic database 310, information such as age, sex, occupation and address of test subjects registered as users of the devices 10 on a test subject side is recorded.

In the test subject life information database 312, information such as clinical histories, exercise habits, and clinical histories of relatives of the registered test subjects is recorded.

The detection data recorded in the measurement gas database 308 is mutually related with the information on the test subjects recorded in the test subject basic database 310 and the test subject life information database 312 on the basis of the test subject identification information transmitted with the detection data from the devices 10 on a test subject side, and is recorded.

In the disease and disease treatment database 314, information on the diseases which affected test subjects, transmitted from the medical facility 302 when the test subjects using the devices 10 on a test subject side underwent diagnosis in the medical facility 302 (FIG. 4) is recorded. Information such as treatment and medication applied to the test subjects, and subsequent transitions of the diseases is also recorded in the disease and disease treatment database 314 in succession. The information recorded in the disease and disease treatment database 314 is recorded by being related with the detection data of defecation gas of the respective test subjects in the measurement gas database 308. In this way, in the disease and disease treatment database 314, “test subject disease data” on the diseases by which a plurality of test subjects using the test subject side devices are affected, acquired from the medical facility, is accumulated, and recorded.

Information on measures which have been taken by the test subjects using the devices 10 on a test subject side to improve states of health is recorded in the health improvement database 316. For example, when a test subject purchased and started to take supplements and healthy food sold by the companies 306 (FIG. 4) recorded in the server 12, or joined sports clubs operated by the companies 306 and started exercise, information on them is transmitted to the server 12 from the respective companies 306, and is recorded in the health improvement database 316. Alternatively, the present invention can be also configured so that the test subject declares the information on the above via the device 10 on a test subject side or the like. The information recorded in the health improvement database 316 is recorded by being related with the detection data of defecation gas of the respective test subjects in the measurement gas database 308.

In the display table database 318, the physical condition display tables (FIG. 8) obtained by analyzing the information recorded in the above described respective databases, and the diagnosis tables (FIG. 10) are recorded. It is preferable that the physical condition display table and the diagnosis table are created for each age and sex of test subjects, and are updated in succession to be suited to each piece of information inputted to the server 12. The server 12 selects the latest physical condition display table which is suited to the test subject who performed measurement of physical condition, from the physical condition display tables recorded in the display table database 318, and allows the display device 68 of the device 10 on a test subject side to display the latest physical condition display table.

Next, with reference to FIG. 15, a procedure of construction of the database 12a will be described.

The flowchart shown in FIG. 15 is executed each time information other than the detection data of defecation gas is inputted to the server 12.

Processing by the flowchart shown in FIG. 15 is executed by a “data analyzer 12b on a server side”, and the data analyzer 12b on a server side is realized by executing a program in the server 12. First, in step S1, it is determined whether or not information inputted to the server 12 relates to the test subject registered in the measurement gas database 308. If the information is not the information on the registered test subject, processing of the flow chart shown in FIG. 15 of one time is ended, because the information cannot be associated with the detection data of defecation gas which is recorded.

If the information is the information on the registered test subject, processing proceeds to step S2, and it is determined whether or not the information inputted to the server 12 is data concerning the disease by which the test subject has been affected. If the information inputted to the server 12 is the data concerning the disease, in step S3 and the following steps, the information is processed as the disease and disease treatment information, and is recorded in the disease and disease treatment database 314.

In step S4, the information which is inputted to the server 12 and recorded in the disease and disease treatment database 314, and the detection data of defecation gas recorded in the measurement gas database 308 are associated with each other by “relating means (circuit)”. The “relating means” is realized by a circuit built in the data analyzer 12b on a server side. For example, when a certain test subject is diagnosed as having colorectal cancer in the medical facility 302, “test subject defecation gas data” which has been previously acquired for the test subject, and recorded in the measurement gas database 308, and information of “test subject disease data” which has been transmitted from the medical facility 302 and recorded in the disease and disease treatment database 314 are associated with each other, and “affected test subject defecation gas data” is generated.

FIG. 16 is an example of the “affected test subject defecation gas data” that is associated with the data of disease in this way. The “affected test subject defecation gas data” shown in FIG. 16 is displayed as plotted points in a table where a vertical axis represents a first index based on wrong physical condition gas “odiferous gas”, and a horizontal axis represents a second index based on a healthy-state gas (hydrogen), similarly to the physical condition display table and the diagnosis table. In this manner, the “affected test subject defecation gas data” is recorded as a time-dependent change characteristic of a correlation of odiferous gas and healthy-state gas. Indexes in the first index are based on the first detection data concerning odiferous gas acquired by the gas detector 20, and indexes in the second index are based on the second detection data concerning hydrogen gas. In FIG. 16, detection data of defecation gas for last ten years is shown, in such a manner that a value obtained by averaging the detection data of defecation gas for last one year from a time point when colorectal cancer was diagnosed is displayed as a plotted point “1”, an average value of the previous one year from the last one year is displayed as a plotted point “2”, an average value of the previous one year but one is displayed as a plotted point “3”, and the like. In the example in FIG. 16, the plotted point moves from a lower right showing a healthy state to an upper left showing wrong physical condition year by year, and colorectal cancer is diagnosed finally.

Next, in step S5 in FIG. 15, a large number of “affected test subject defecation gas data” are totalized, a reference value thereof is generated and recorded. That is, each time information on the disease of a test subject is inputted, the “relating means” of the data analyzer 12b on a server side associates information of the disease with the “test subject defecation gas data” recorded in the measurement gas database 308, and generates a large number of “affected test subject defecation gas data” as shown in FIG. 16. The “relating means” classifies the “affected test subject defecation gas data” of the respective test subjects affected by the same disease into a plurality of groups on the basis of the information of the test subjects recorded in the test subject basic database 310, and totalizes the “affected test subject defecation gas data” for each of the groups. For example, the “relating means” divides the test subjects affected by colorectal cancer into groups for each age on the basis of the ages recorded in the test subject basic database 310, and calculates an average value of the “affected test subject defecation gas data” for each of the groups. The groups can be classified on the basis of age, sex, residential district, occupation, living environment or the combination of these items.

FIG. 17 is an example of “reference affected test subject defecation gas data” obtained by averaging the “affected test subject defecation gas data” for each of groups according to ages, FIG. 17A is a result of totalization for a group of fifties, and FIG. 17B is a result of totalization for a group of twenties. As is understandable from FIG. 17, even with the same disease, time-dependent change characteristic of the defecation gas data differs depending on the ages in which the test subjects are affected. In this way, according to the present invention, by accumulating a large number of “test subject defecation gas data” and “test subject disease data” in the server 12, it becomes possible to divide the test subjects affected by the same disease into small groups, and it becomes possible to generate more precise “reference affected test subject defecation gas data”. In this way, the “reference affected test subject defecation gas data” is created on the basis of the “test subject defecation gas data” of a plurality of test subjects affected by the same disease, and is made a reference concerning the risk of being affected by the disease. It is preferable to create the “reference affected test subject defecation gas data” for each of predetermined groups of test subjects for the same disease. The “relating means” evaluates “accuracy” of the “reference affected test subject defecation gas data” on the basis of the number of “affected test subject defecation gas data” used for creating the “reference affected test subject defecation gas data”, a variation in the data and the like, and records the “accuracy”. The “accuracy” is notified together when the affection risk of the disease is notified to a test subject or the like.

In the above described example, the “test subject defecation gas data” is associated with colorectal cancer which is the disease assumed to be diagnosed on the basis of the detection data of defecation gas, however, it is also possible to generate “affected test subject defecation gas data” for an arbitrary disease, and totalize the “affected test subject defecation gas data”. By providing the data collected in this way to the respective research institutions 304 to perform analysis, a relationship between defecation gas data and a new disease can be clarified, and it becomes possible to utilize the present invention in diagnosis of various diseases.

Next, in step S6 in FIG. 15, the information of treatment performed for a specific disease is recorded. That is, disease treatment information such as medical treatment and medication performed for the specific disease is transmitted to the server 12 from the medical facility 302. The “relating means” can also perform totalization about the treatment to the disease like this. In this way, by totalizing the “test subject defecation gas data” for each treatment adopted for the same disease, it becomes possible to grasp how the disease is improved by the treatment, and it becomes possible to recognize the effects of medical treatment and medication.

Meanwhile, if it is determined that the information inputted to the server 12 is not the data concerning the disease which affects the test subject in step S2 in FIG. 15, processing proceeds to step S7. In step S7 and the following steps, processing in a case of the inputted information being health improvement information of the test subject is executed. Information other than the disease of the test subject, inputted to the server 12, includes information that the test subject starts to take supplements and health food, information that a test subject joins a sports club to start exercising and the like.

In step S8, a measure for health improvement started by the test subject is associated with the “test subject defecation gas data” of the test subject by the “relating means”.

Next, in step S9, an effect of the health improvement measure is analyzed and recorded on the basis of the health improvement measure and the test subject defecation gas data which are associated in step S8. That is, if the time-dependent change characteristic of the test subject defecation gas data and the health improvement measure taken by the test subject are associated with each other, the effect of the measure can be grasped. For example, if healthy-state gas in defecation gas increases, and odiferous gas starts to decrease, after a certain test subject starts taking a supplement, it can be verified that harmful intestinal bacteria decreases, good intestinal bacteria increases and the like owing to intake of the supplement. If the interval of defecation changes to be regular after intake of the supplement is started, it can be verified that constipation is being relieved by the supplement. In this way, by associating the time-dependent change characteristic of the test subject defecation gas data with supplements, health food, medicines, dietary habits, exercise habits and the like, the effect of the measures for health can be verified.

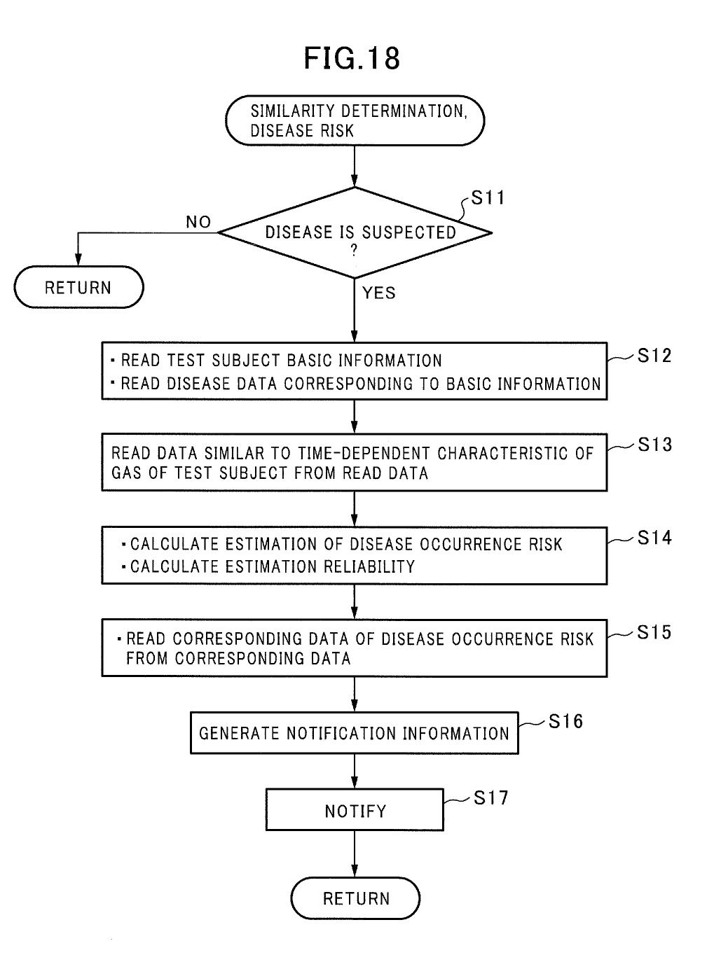

Next, with reference to FIG. 18 to FIG. 23, similarity determination and notification of a risk of a disease by the server side data analyzer will be described.

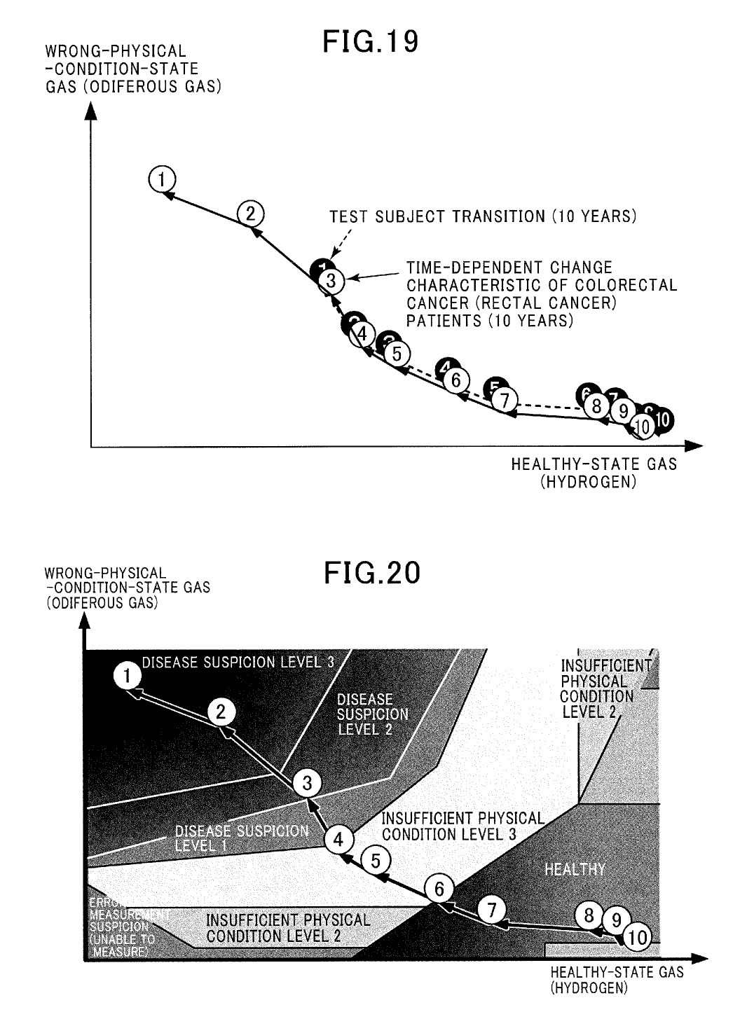

FIG. 18 is a flowchart showing a procedure of similarity determination and notification by similarity determination means (circuit) built in the server side data analyzer. FIG. 19 shows an example of “reference affected test subject defecation gas data” and “test subject defecation gas data” having similarity. In the present embodiment, “reference affected test subject defecation gas data” and “test subject defecation gas data” are respectively expressed as time-dependent change characteristics of a correlation of odiferous gas and healthy-state gas, and similarity of them is determined.

The flowchart shown in FIG. 18 is executed by “similarity determination means” realized as a circuit in the data analyzer 12b on a server side at intervals of about several months to a year for each of individual test subjects. First, in step S11 in FIG. 18, it is determined whether or not “test subject defecation gas data” of a test subject is in a state where a disease should be suspected, by the “similarity determination means”. When it is in a state where no disease is suspected, processing of the flowchart shown in FIG. 18 of one time is ended. That is, when the physical condition of the test subject measured is relatively healthy, even if the time-dependent change characteristic of the “test subject defecation gas data” is similar to a part of “reference affected test subject defecation gas data” of a certain disease, the probability of the following time-dependent change becoming similar to “reference affected test subject defecation gas data” is low, and the time-dependent change often heads to recovery. Accordingly, if similarity to specific “reference affected test subject defecation gas data” is notified in the state where the physical condition of a test subject is not so bad, an unnecessary mental burden is applied to the test subject. Consequently, in the state where the physical condition of a test subject is not so bad, the similarity of the time-dependent change characteristic of “test subject defecation gas data” and the time-dependent change characteristic of “reference affected test subject defecation gas data”, in the correlation of odiferous gas and healthy-state gas, is not determined.

Meanwhile, if the “test subject defecation gas data” of a test subject is in the state where a disease is suspected, processing proceeds to step S12. In step S12, the test subject basic information of the test subject is read from the test subject basic database 310, and “reference affected test subject defecation gas data” for the same group as the test subject is read. That is, the “reference affected test subject defecation gas data” which is obtained by totalizing defecation gas data of a plurality of test subjects belonging to the group of the same age, sex and the like as those of the test subject is read. In the diagnosis table (FIG. 10), a plurality of physical condition stages from a healthy state to a state with concern for disease are set, and in the present embodiment, when the latest “test subject defecation gas data” is deteriorated to a predetermined physical condition stage, it is determined as a state where a disease is suspected, and determination of similarity is executed.

Next, in step S13, data similar to a time-dependent change characteristic of the “test subject defecation gas data” of the test subject is selected from the “reference affected test subject defecation gas data” read in step S12. In step S14, the “similarity determination means” compares the time-dependent change characteristic of the selected “reference affected test subject defecation gas data” and the time-dependent change characteristic of the “test subject defecation gas data”, and calculates the risk of the test subject being affected by the disease thereafter and reliability of the estimation.

FIG. 19 shows an example of “test subject defecation gas data” similar to “reference affected test subject defecation gas data”. In the example shown in FIG. 19, the time-dependent change of “test subject defecation gas data” of a test subject is extremely similar to the time-dependent change characteristic of the defecation gas data in an average test subject affected by colorectal cancer, in the correlation of odiferous gas and healthy-state gas, and the latest data (a plotted point “1”) of the test subject substantially overlaps a plotted point “3” of “reference affected test subject defecation gas data”. Consequently, it can be determined that there is a high risk of the test subject being affected by colorectal cancer in two to three years ahead if the test subject follows a similar time-dependent change to the “reference affected test subject defecation gas data”.

Next, in step S15, data of a test subject having a time-dependent change characteristic similar to the “test subject defecation gas data”, and thereafter having physical condition recovered is retrieved from the measurement gas database 308, and at the same time, data of a health improvement measure taken by the test subject having the physical condition recovered is retrieved from the disease and disease treatment database 314 and the health improvement database 316.

In step S16, useful information to be notified to the test subject is generated by the data analyzer 12b on a server side, on the basis of the data retrieved in steps S14 and S15. Subsequently, in step S17, the notification information generated in step S16 is notified, and the processing of the flow chart in FIG. 18 of one time is ended.

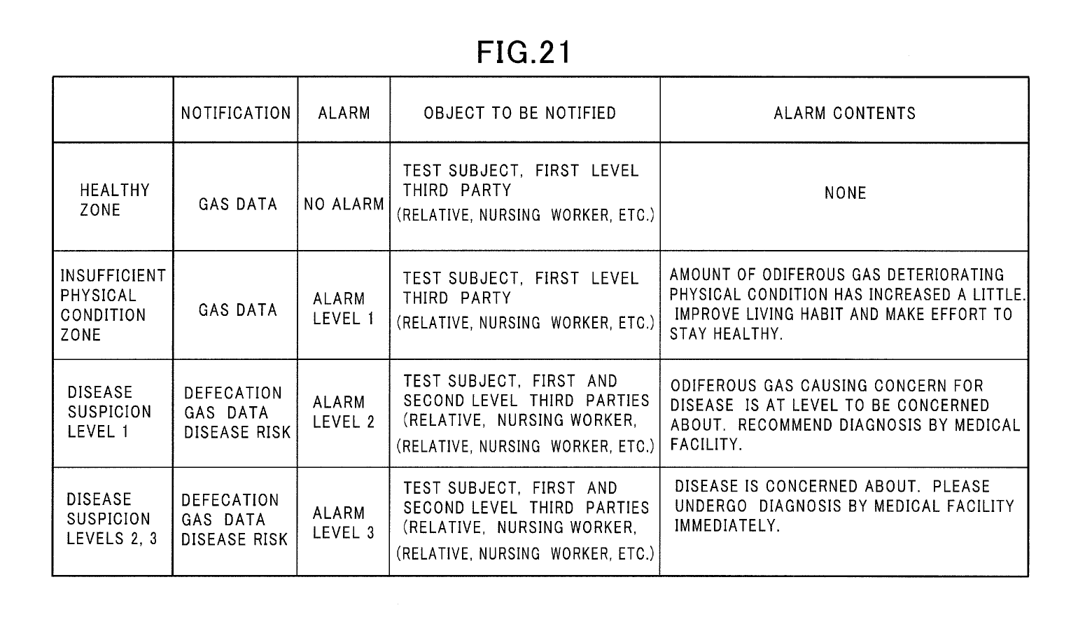

Next, with reference to FIG. 20 and FIG. 21, notification by notification means built in the data analyzer 12b on a server side will be described. The display device and the speaker connected to the server 12 used as the data analyzer 12b on a server side function as notification means which notifies the users of the server of various kinds of information.

FIG. 20 shows physical condition of a test subject in the diagnosis table. FIG. 21 shows a mode of notification which is performed in accordance with the physical condition of a test subject.

First, when the latest physical condition stage to which the test subject belongs is in a zone of “healthy” in FIG. 20 (for example, when the latest plotted point is located in positions of plotted points “7” to “10” in FIG. 20), similarity determination by the “similarity determination means” is not executed because the physical condition stage is in a state where no disease is suspected. In this state, as described by FIG. 7 and FIG. 8, the information displayed in the display device 68 of the device 10 on a test subject side only relates to data of defecation gas, and a risk and the like of a specific disease is not notified (displayed). Further, a person who is notified is only a test subject, and if the test subject stays in a nursing facility or the like, a nursing worker, or a relative of the test subject is given notification.

If the latest physical condition stage to which a test subject belongs is in a zone of “insufficient physical condition” in FIG. 20 (for example, if the latest plotted point is located in the positions of plotted points “5” to “6” in FIG. 20), it is not highly possible that the physical condition of the test subject deteriorates as it is and is affected by a specific disease. Consequently, no risk of being affected by a specific disease is notified, and only the plotted points concerning the data of defecation gas (for example, screens in FIG. 7 and FIG. 8) are displayed in the physical condition display table, and are notified. In this way, when the latest physical condition stage of the test subject is in a relatively healthy state, it is notified whether or not the physical condition is improved as “health care information” instead of an affection risk. Further, if the physical condition of the test subject is in a zone of “insufficient physical condition”, an alarm, “Amount of odiferous gas deteriorating physical condition has increased a little. Improve your living habit to stay healthy.” or the like is displayed in the display device 68 of the device 10 on a test subject side to encourage the test subject to improve the living habit. A person who is notified is only a test subject, and if the test subject stays in a nursing facility or the like, a nursing worker or a relative of the test subject is given notification.

If the latest physical condition stage to which a test subject belongs is in a zone of “disease suspicion level 1” in FIG. 20 (for example, when the latest plotted point is in a position of a plotted point “4” in FIG. 20), a risk of the test subject being affected by a specific disease has increased, and therefore the risk of being affected by a specific disease is notified together with the information of defecation gas data. For example, an alarm indicating that “Odiferous gas causing concern for a disease is at a level to be worried about. Please undergo diagnosis by a medical facility.” or the like is displayed in the display device 68 of the device 10 on a test subject side. In this case, as persons notified, besides the test subject, a nursing worker or a relative of the test subject in the case where the test subject stays in a nursing facility or the like, the medical facility which the test subject has registered, and an employer of the test subject are also notified. In this way, in the biological information measurement system 1 of the present embodiment, the notification mode and the persons notified (including organizations such as a medical facility and a company) differ in accordance with the physical condition stage to which the latest test subject defecation gas data belongs.

If the latest physical condition of a test subject is in zones of “disease suspicion levels 2, 3” in FIG. 20 (for example, when the latest plotted point is located in positions of plotted points “1” to “3” in FIG. 20), a risk of the test subject being affected by a specific disease has further increased, and therefore the risk of being affected by the specific disease as well as the information of defecation gas data is notified. For example, an alarm indicating that “Disease is suspected. Please undergo diagnosis by a medical facility urgently.” or the like is displayed in the display device 68 in the device 10 on a test subject side. In this case, as the persons to be notified, besides the test subject, a nursing worker or a relative of the test subject in the case of the test subject staying in a nursing facility or the like, the medical facility which the test subject has registered, and the employer of the test subject are also notified.

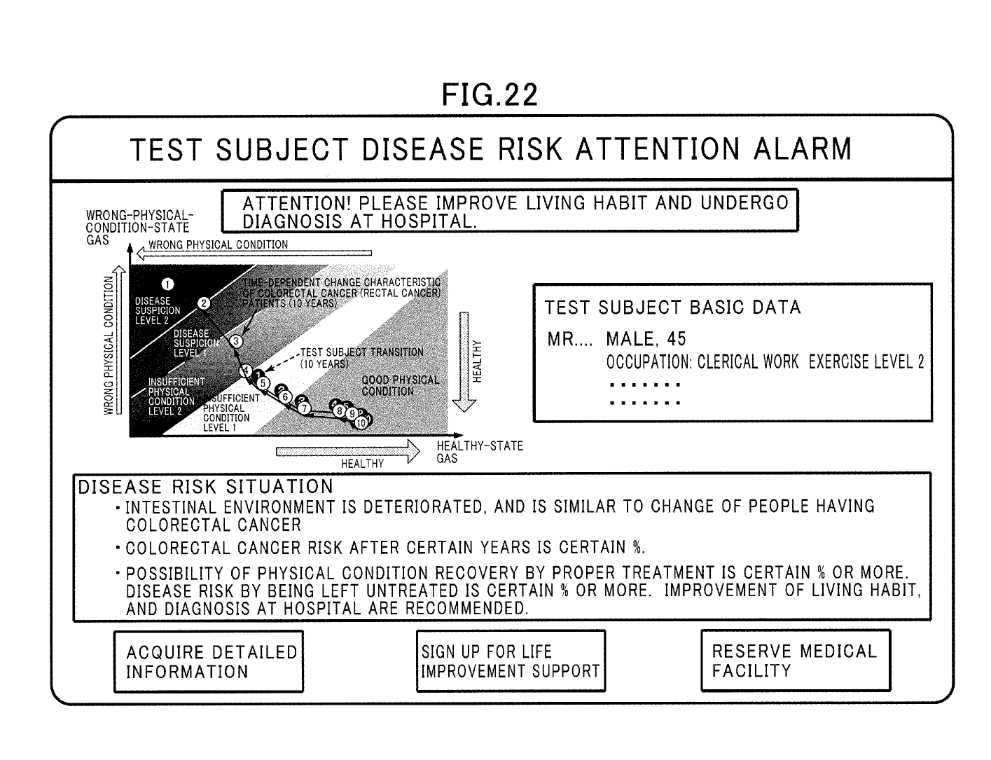

Next, with reference to FIG. 22, a risk display screen presented to a test subject and the like will be described.

FIG. 22 is an example of the risk display screen displayed in the display device 68 in the device 10 on a test subject side. The risk display screen is displayed by pressing a button of “disease risk display” displayed on the screen in FIG. 8, for example. In the risk display screen shown in FIG. 22, the time-dependent change characteristic of “test subject defecation gas data”, and the time-dependent change characteristic of “reference affected test subject defecation gas data” read in step S13 in FIG. 18 are displayed in the physical condition display table, and similarity of them is shown, besides the basic data of the test subject. This provides a strong motivation for improving physical condition, because the test subject recognizes that the time-dependent change of the physical condition of himself or herself is very similar to the time-dependent change of the other test subjects who were affected by colorectal cancer or the like several years later.

Under the physical condition display table, as a disease risk situation, alarms such as “Intestinal environment is deteriorated, and is similar to the change of people having colorectal cancer.”, “Risk of colorectal cancer after certain years is a certain percent.”, “Possibility of recovering physical condition by proper treatment is a certain percent or more. A risk of disease when no treatment is given is a certain percent or more.”, and “Recommend improvement of a living habit, and diagnosis in hospital.” are displayed (notified). Further, under these alarms, buttons such as “Acquire detailed information”, “Sign up for life improvement support”, and “Reserve medical facility” are displayed. In this way, as the physical condition stage of the test subject is closer to a state having concern for a disease, the analysis result of the physical condition is notified in more detail, and the disease which might affect the test subject, and the risk of the test subject being affected by the disease after a predetermined period (the period in which the risk of being affected increases) are also notified.

When a test subject presses the button of “Acquire detailed information”, information such as accuracy of “reference affected test subject defecation gas data” determined as similar to the “test subject defecation gas data” of himself or herself in the time-dependent change characteristic, and precision of the similarity is displayed. The accuracy of “reference affected test subject defecation gas data” is set at a higher value as the number of data of test subjects used for generating the “reference affected test subject defecation gas data” is larger, and a variation in data is smaller. Precision of the similarity is set at a higher value as the time-dependent change characteristic of the “test subject defecation gas data” of himself or herself, and the time-dependent change characteristic of the “reference affected test subject defecation gas data” are close to each other for a longer period. Even if “test subject defecation gas data” and “reference affected test subject defecation gas data” are similar to each other, if “accuracy” of the “reference affected test subject defecation gas data” is low, it is possible that an unnecessary mental burden is applied to the test subject by notification of an erroneous risk of disease. Consequently, it is preferable to change a content which is notified to a test subject, and a timing for notifying, in accordance with the “accuracy” of “reference affected test subject defecation gas data”. For example, the notification means can be also configured so that a specific disease risk is notified at a stage where a physical condition stage becomes worse in a case where accuracy is low than in a case where the accuracy is high.

If a test subject presses the button of “Sign up for life improvement support”, “risk reduction information” which the other test subjects in physical condition similar to the test subject have used to find recovery of the physical condition is displayed. More specifically, information on supplements, health food, medicines, sports facilities and the like which are useful as measures for reducing the risk of the test subject being affected is displayed. For the information displayed at this time, “risk reduction information”, which is confirmed to provide a high effect of improvement of physical condition (a high risk reduction effect) by analysis in the data analyzer 12b on a server side, is preferentially displayed. Accordingly, the test subject can obtain truly useful information to recover the physical condition of himself or herself.

If a test subject presses the button of “Reserve a medical facility”, a reservation screen of the medical facility which the test subject has registered, or a recommendable medical facility selected in the data analyzer 12b on a server side is displayed. Accordingly, it is possible for the test subject to undergo diagnosis in the medical facility quickly, and have more accurate diagnosis and effective treatment.

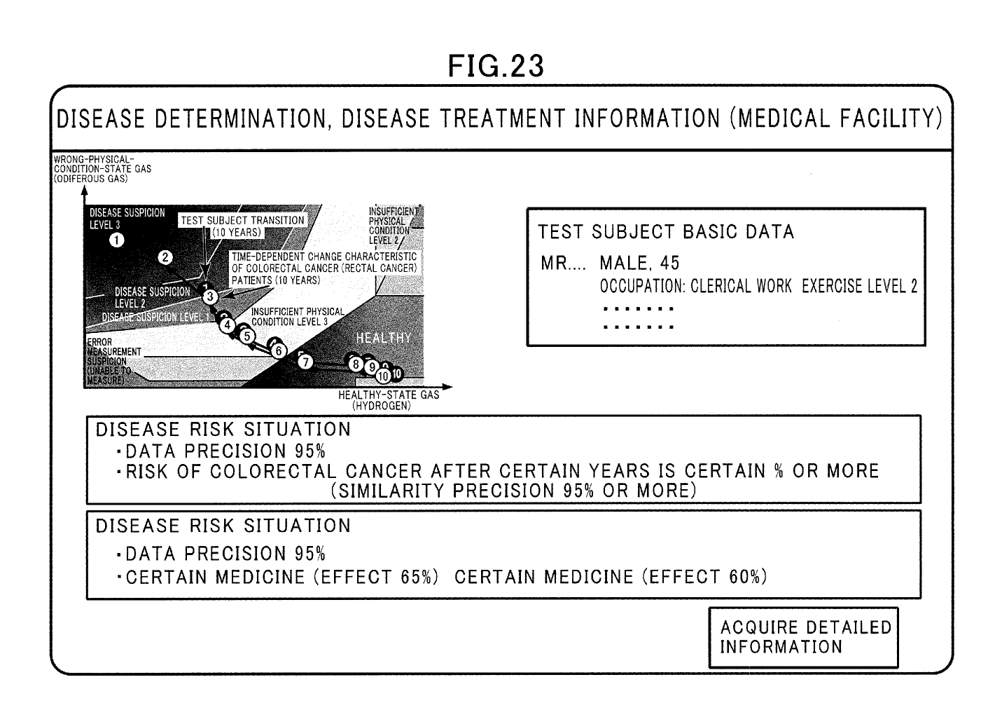

Next, with reference to FIG. 23, a disease determination screen displayed in a terminal of a medical facility for a doctor and the like of the medical facility to refer to when the test subject notified of the risk of a disease undergoes diagnosis in the medical facility will be described.

As shown in FIG. 23, in the disease determination screen, the time-dependent change characteristic of “test subject defecation gas data” and the time-dependent change characteristic of “reference affected test subject defecation gas data” are displayed in a diagnosis table, besides the basic data of the test subject, similarly to the risk display screen. Under the time-dependent change characteristics, information on the accuracy of the “reference affected test subject defecation gas data”, the similarity to “test subject defecation gas data” and the like is displayed as a “disease risk situation”. Under the “disease risk situation”, effects of a plurality of medicines given to patients (test subjects) which have been in similar physical condition and the like are displayed as “disease risk reduction information”. Accordingly, a doctor can grasp the physical condition of the test subject in a time-dependent manner, can obtain information of the medicines which have been effective for the patients having similar symptoms, and can make use of the information for diagnosis and determination of a medical treatment plan. By pressing a button of “Acquire detailed information” displayed at a lower end of the disease determination screen, a doctor or the like can obtain more detailed information of defecation gas of the test subject who have undergone diagnosis.

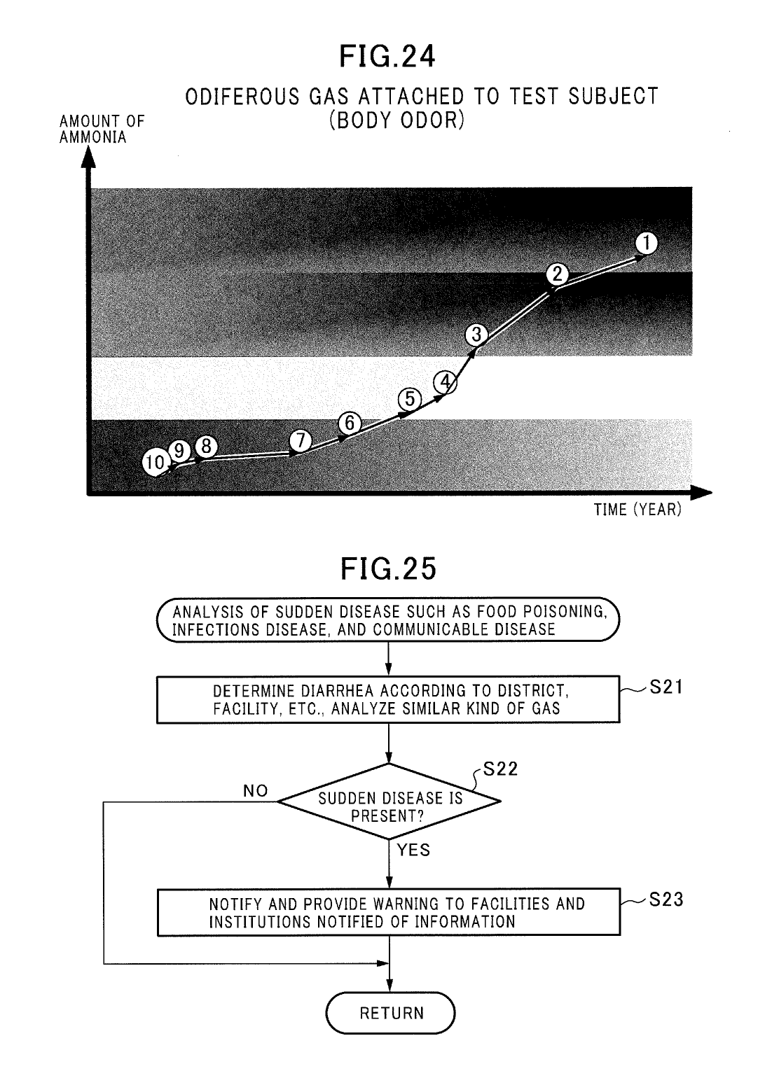

Next, with reference to FIG. 24, measurement of physical condition by detection of gas attached to a test subject will be described. FIG. 24 is a graph showing a time-dependent change of gas attached to a test subject such as ammonia.

As described with reference to FIG. 11, in the biological information measurement system 1 of the embodiment of the present invention, the gas detector 20 also detects odiferous gas (odiferous gas attached to a test subject) before the test subject sits on the seat 4, after the test subject entered the toilet installation room. The odiferous gas detected in this period contains information on a body odor of the test subject, although the odiferous gas is also influenced by odiferous gas remaining in the toilet installation room, a perfume attached to the test subject and the like. It is possible to remove the influence of the odiferous gas remaining in the toilet installation room by subtracting a noise level of the odiferous gas noise detected before the test subject enters the toilet installation room.

Here, if a test subject has a liver disease, an amount of ammonia emitted as a body odor greatly increases, and therefore it is possible to find the disease at an early stage by measuring the body odor of the test subject in a time-dependent manner for a long period. The amount of odiferous gas attached to a test subject detected before the test subject sat on the seat can be displayed as an option, together with the measurement result of defecation gas. If the body odor displayed in this way has continued to increase for a long period as shown in FIG. 24, and the test subject does not have a habit of using a perfume or the like, there is concern for a liver disease. The device 10 on a test subject side also transmits the detection data of the odiferous gas attached to the test subject to the server 12, and the data analyzer 12b on a server side also utilizes the detection data of the odiferous gas attached to the test subject in analysis of physical condition.

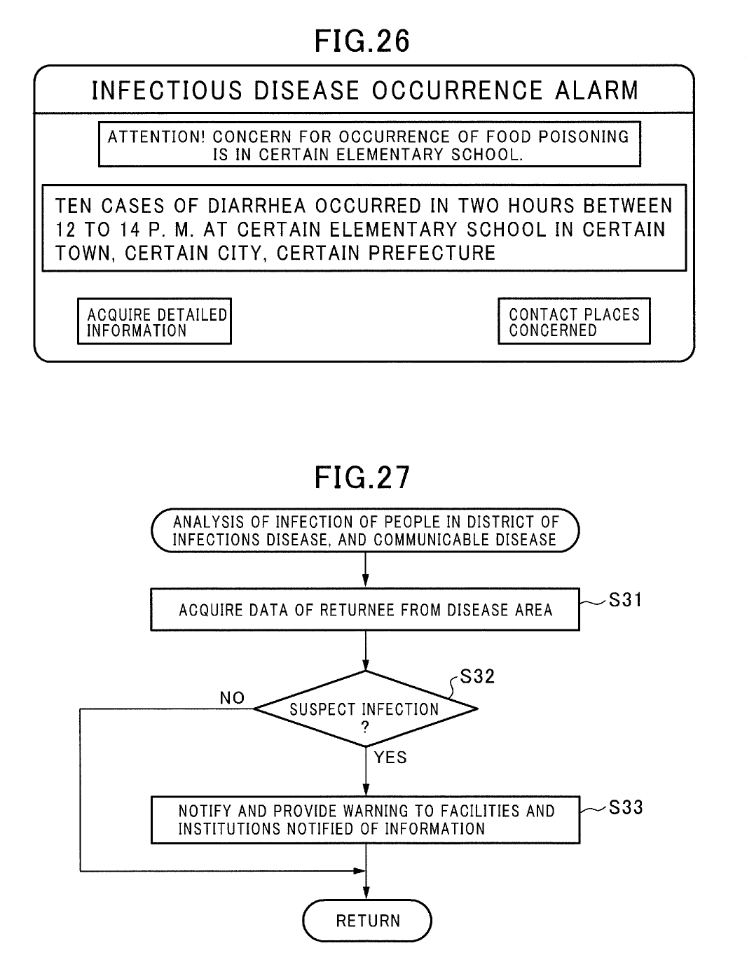

Next, with reference to FIG. 25 and FIG. 26, detection of a disease which suddenly prevails, by the biological information measurement system 1 of the embodiment of the present invention will be described. FIG. 25 is a flowchart for detection of a disease that suddenly prevails. FIG. 26 shows an example of a notification screen in a case of prevalence of a disease being detected.

As described above, the device 10 on a test subject side is provided with a microwave sensor as the defecation/urination detection sensor 38 (FIG. 2), and thereby can detect diarrhea and the like of a test subject. Information on diarrhea and the like of a test subject detected by the defecation/urination detection sensor 38 which functions as diarrhea detection means (circuit) in this way is transmitted from the device 10 on a test subject side to the server 12, with the information on defecation gas.

For example, if mass food poisoning occurs due to school meals or the like, the number of patients (test subjects) having diarrhea suddenly increases, in a certain district where the students of the school are living. The data analyzer 12b on a server side detects a sudden increase in the number of test subjects having diarrhea in a specific district like this, and notifies organizations concerned of the sudden increase in diarrhea.

First, in step S21 in FIG. 25, the information on diarrhea of test subjects transmitted from the respective devices 10 on a test subject side is totalized in a predetermined district, predetermined facilities or the like at each predetermined time. Next, in step S22, it is determined whether or not the totalized number of occurrences of diarrhea is a predetermined threshold value or more. If the number of occurrences of diarrhea is less than the predetermined threshold value, processing by the flowchart in FIG. 25 of one time is ended. If the number of occurrences of diarrhea is the predetermined threshold value or more, processing proceeds to step S23, and in step S23, the data analyzer 12b on a server side notifies a public health care center, a ward office or facilities concerned in the district, where occurrence of food poisoning or the like is suspected. In this way, the data analyzer 12b on a server side analyzes a prevailing situation of a disease on the basis of the information on diarrhea transmitted from the respective devices 10 on a test subject side, and allows the notification means to notify that the disease is prevalent, when the data analyzer 12b on a server side determines that the disease is prevalent.

FIG. 26 is an example of the notification screen which notifies occurrence of mass food poisoning or the like as above. The notification screens as in FIG. 26 are displayed in terminals of the public institutions 307a (FIG. 5) such as municipalities, the police, fire departments or a public health care center of the district where there is concern for occurrence of mass food poisoning or the like. The public institutions 307a receiving notification like this can utilize the biological information measurement system 1 of the present embodiment in determination of presence or absence of occurrence of mass food poisoning, and analysis of the cause. Further, by obtaining detailed information from the terminals of the public institutions 307a, it becomes possible to analyze the situation of occurrence of food poisoning or the like, and instruct preparation for treatment to the medical facilities in the district. By notifying public health care centers and the like around the district, it is possible to take preventive measures against spread of infection.

Next, with reference to FIG. 27, prevention of an epidemic of an infectious disease, by the biological information measurement system 1 of the embodiment of the present invention will be described. FIG. 27 is a flowchart for prevention of an epidemic of an infectious disease.

For example, when a traveler or the like returns home from an area where an infectious disease which causes symptoms of diarrhea or the like to appear is prevalent, the test subject identification information of the test subject returning home is recorded in an airport, the harbor 307b (FIG. 5) or the like. Next, in step S31 in FIG. 27, defecation gas data and data on diarrhea transmitted to the server 12 is acquired, together with the test subject identification information recorded as the test subject who has returned home. In step S32, it is determined whether or not a symptom such as diarrhea appears, and if the symptom does not appear, the processing of the flowchart in FIG. 27 of one time is ended. Monitoring of the defecation gas data and the like for the test subject who has returned home is repeatedly executed at predetermined intervals for a sufficiently longer period than a period of incubation of the infectious disease with which the test subject might be infected. If a symptom such as diarrhea appears, processing proceeds to step S33 to notify the public health care center in the district of residence of the test subject who has returned home of the appearance of the symptom, and necessary measures such as isolation is urged. Accordingly, it becomes possible to monitor the state of health of the test subject who is suspected to be infected with the infectious disease in detail, and to take preventive measures of an epidemic of the infections disease at an early stage.

By acquiring and monitoring the defecation gas data and the data on diarrhea transmitted from the test subject living in a specific district, the situation of prevalence of an infectious disease such as viral gastroenteritis in the district can be grasped, and it becomes possible for the public health care center 307a or the like which is notified to take proper measures and steps. In this way, the data analyzer 12b on a server side notifies a specific person of the municipalities, the public health care center or the like set in advance, or an agency, of the information on the specific test subject set in advance or a test subject living in a specific district, which is acquired and transmitted by the device 10 on a test subject side.

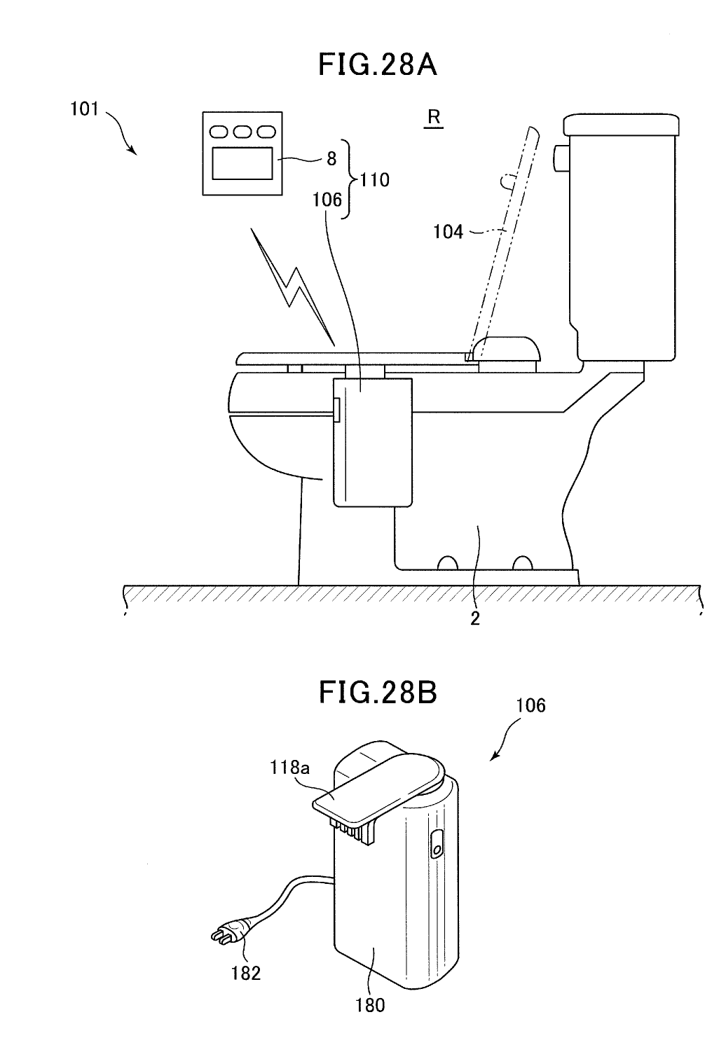

Next, with reference to FIG. 28, a biological information measurement system according to a second embodiment of the present invention will be described.

Although in the biological information measurement system of the first embodiment described with reference to FIG. 1, it is described that the measuring device 6 is assembled inside the seat 4 mounted on the flush toilet 2 installed in the toilet installation room R, the measuring device is not required to be always assembled inside the seat in the biological information measurement system of the present invention.

FIG. 28A shows a state in which a test subject side device of a biological information measurement system in accordance with a second embodiment is attached to a flush toilet installed in a toilet installation room, and FIG. 28B is a perspective view showing a measuring device of the test subject side device shown in FIG. 28A. The second embodiment is only different in a configuration of the test subject side device as compared with the first embodiment. As shown in FIG. 28A, a biological information measurement system 101 of the present embodiment has the same configuration as that of the first embodiment, except that only a measuring device 106 of a device 110 on a test subject side is different. The measuring device 106 of the present embodiment is provided separately from a seat 104.

As shown in FIG. 28B, the measuring device 106 includes a device body 180, a duct 118a that is attached on a top face of the device body 180 so as to extend in a traverse direction, and that is provided with an edge portion bent downward, and a power source code 182 that is connected to the device body 180. As shown in FIG. 28A, the measuring device 106 is fixed while an end of the duct 118a is positioned in the bowl by hanging the edge portion of the duct 118a on a sidewall of a bowl of the flush toilet 2.

The device body 180, as with the first embodiment, includes a hydrogen gas sensor, an odiferous gas sensor, a carbon dioxide sensor, a humidity sensor, a temperature sensor, an entrance detection sensor, a seating detection sensor, a defecation/urination detection sensor, a suction device, a sensor heater, and a transmitter-receiver. Gas sucked through the duct 118a is deodorized and is discharged through a deodorized air outlet provided in a bottom face of the device body 180. In the duct 118a, there are provided the hydrogen gas sensor, the odiferous gas sensor, the carbon dioxide sensor, the humidity sensor, the temperature sensor, the sensor heater, and a fan. Arrangement of the sensors in the duct 118a is the same as that of the first embodiment, so that description thereof is omitted. According to this kind of configuration, the measuring device 106 of the present embodiment is also capable of acquiring detection data corresponding to the amount of odiferous gas, hydrogen gas, and carbon dioxide, contained in defecation gas, by using the odiferous gas sensor, the hydrogen gas sensor, and the carbon dioxide sensor.

It is desirable that the seat 104 to be used along with the measuring device 106 of the present embodiment is a seat with a cleaning function that includes a toilet lid opening/closing device, a nozzle driving device, a nozzle cleaning device, a toilet cleaning device, and a toilet disinfection device, the seat being capable of communicating with the measuring device 106. Using the measuring device 106 along with this kind of seat enables various cleaning operations and disinfecting operation to be performed when stink gas is detected.

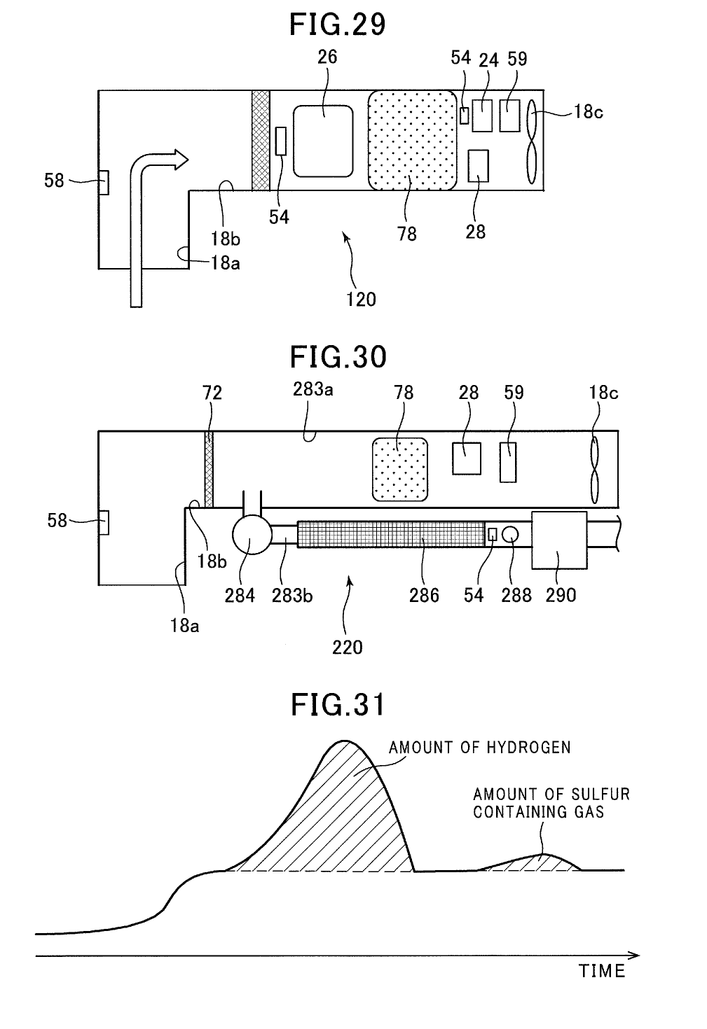

Next, with reference to FIG. 29, a biological information measurement system according to a third embodiment of the present invention will be described.

Although in the first embodiment, the gas detector 20 is configured so that the hydrogen gas sensor 24 is provided downstream of the deodorant filter 78, as shown in FIG. 3, this kind of configuration is not always required. FIG. 29 shows a configuration of a gas detector provided in a biological information measurement system of a third embodiment. The third embodiment is only different in a configuration of the gas detector as compared with the first embodiment. As shown in FIG. 29, arrangement of the hydrogen gas sensor 24 in the gas detector 120 in the present embodiment is different from that in the embodiment shown in FIG. 3. In the present embodiment, the hydrogen gas sensor 24 is provided downstream of the deodorant filter 78 in the air intake passage 18b. According to this kind of configuration, even if a sensor sensitive to odiferous gas as well as to hydrogen gas is used as the hydrogen gas sensor 24, it is possible to remove influence of odiferous gas from data to be outputted from the hydrogen gas sensor 24.

Next, with reference to FIG. 30 and FIG. 31, a biological information measurement system according to a fourth embodiment of the present invention will be described.

In the first embodiment, although a detection value of odiferous gas is calculated by subtracting a detection value acquired by the hydrogen gas sensor 24 from a detection value acquired by the odiferous gas sensor 26 to separate influence of hydrogen gas, the present invention is not limited to the way above. For example, as described below, influence of hydrogen gas can be also separated by varying a reaching time of each of hydrogen gas and odiferous gas to the odiferous gas sensor 26.

FIG. 30 shows a configuration of a gas detector of a fourth embodiment, the gas detector being configured to vary a reaching time of each of hydrogen gas and odiferous gas to the odiferous gas sensor to separate influence of the hydrogen gas. The fourth embodiment is only different in the configuration of a gas detector as compared with the first embodiment. As shown in FIG. 30, in the present embodiment, there is provided a branch passage 283b that branches from a main passage 283a of the air intake passage 18b in the duct 18a. While a hydrogen gas sensor and an odiferous gas sensor are separately provided in the first embodiment, the present embodiment is configured to detect both hydrogen gas and odiferous gas by using one semiconductor gas sensor.

As with the first embodiment, the air intake passage 18b includes the filter 72, the deodorant filter 78 provided downstream of the filter 72, and the suction fan 18c, and the branch passage 283b branches on the downstream side of the filter 72. The filter 72 does not have a deodorizing function, and allows odiferous gas and hydrogen to pass therethrough, but prevents foreign material, such as urine, and a cleaner from passing therethrough. As with the first embodiment, the deodorant filter 78 is also a catalyst that adsorbs gas components of odiferous gas or the like.

Defecation gas in the bowl 2a of the toilet is sucked into the air intake passage 18b at a fixed flow rate by the suction fan 18c. The defecation gas sucked into the air intake passage 18b passes through the filter 72 so that foreign material, such as urine, and a cleaner, is removed, and then is returned into the bowl 2a of the toilet after gas components of odiferous gas or the like are removed by the deodorant filter 78.

The branch passage 283b includes a flow channel changeover valve 284, a column 286, a semiconductor gas sensor 288, and a pump 290, in order from an upstream side toward a downstream side.

The flow channel changeover valve 284 is opened in a partial time (a very short time) during an excretory act to allow a part of defecation gas flowing through the air intake passage 18b (for the partial time during the excretory act of a test subject) to be drawn into the branch passage 283b. The flow channel changeover valve 284 is provided at the most upstream portion of the branch passage 283b.

The column 286 is provided downstream of the flow channel changeover valve 284, and is formed by filling elongated piping with thin fibers and the like, for example. The column 286 has a mechanism in which passing time of gas varies in accordance with molecule size (molecular weight), according to a principle of gas chromatography.

The sensor heater 54 is provided upstream of the semiconductor gas sensor 288 to heat a detecting portion of the semiconductor gas sensor 288 to a predetermined temperature as well as remove stink gas components attached to the semiconductor gas sensor 288.

The flow channel changeover valve 284 allows defecation gas in trace amounts flowing through the air intake passage 18b after passing through the filter 72 to flow into the branch passage 283b. Then, when the pump 290 is driven, each of hydrogen and odiferous gas, contained in the defecation gas, passes through the column 286 for a different time in accordance with molecular weight, according to the principle of gas chromatography, to reach the semiconductor gas sensor 288. That is, hydrogen with a small molecular weight tends to easily pass through the column 286 to reach the semiconductor gas sensor 288 in a short time, and odiferous gas with a large molecular weight tends to be difficult to pass through the column 286 to reach the semiconductor gas sensor 288 in a longer time as compared with the hydrogen. The pump 290 is configured to suck defecation gas at a fixed flow velocity.

FIG. 31 shows a detection waveform acquired by a semiconductor gas sensor of a gas detector, shown in FIG. 30. As shown in FIG. 31, according to a configuration of a gas detector 220 of the present embodiment, the semiconductor gas sensor 288 reacts to hydrogen gas and odiferous gas, which are temporally separated. In particular, an excretory act is performed in a short time, and defecation gas containing hydrogen and odiferous gas is also discharged only in a short time. In this way defecation gas is discharged in a short time, and thus providing the column 286 upstream of the semiconductor gas sensor 288 enables a time by which each of hydrogen gas and odiferous gas reaches the semiconductor gas sensor to be varied, whereby it is possible to detect the amount of hydrogen gas, and the amount of odiferous gas, by using one semiconductor gas sensor 288. This is also based on technical findings made by the present inventors that if a method of determining physical condition using a correlation between healthy-state gas and odiferous gas without measuring all of the amount of methyl mercaptan gas in correlation with cancer is adopted, gas only in a specific period can be measured in this kind of method. If a reduction sensor is used, the sensor is inexpensive but it is difficult to separate a large amount of hydrogen contained in defecation gas. In contrast, since the present embodiment allows a small amount of gas to be measured only in a specific period, separation of hydrogen becomes easy so that practicality can be achieved with a very inexpensive sensor.

While the present embodiment allows the column 286 to vary a reaching time of each of hydrogen and odiferous gas to the semiconductor gas sensor 288, it is a matter of course that it is possible to vary a reaching time of methane contained in defecation gas. Accordingly, it is also possible to separate influence of not only hydrogen but also methane from detection data acquired by a semiconductor gas sensor.

According to the biological information measurement system of the embodiment of the present invention, it is possible to notify a risk of a disease such as colorectal cancer at a stage of ahead-disease, by determination of similarity (FIG. 19) of the time-dependent change characteristic of the test subject defecation gas data transmitted from a test subject, and the time-dependent change characteristic of the affected test subject defecation gas data based on the information of a large number of test subjects accumulated in the database 12a in the server 12. In the present embodiment, the risk is not evaluated by defecation gas data of one time, but similarity is estimated on the basis of the time-dependent change characteristics, so that the precision of prediction of the risk can be extremely increased. It becomes possible to notify affection risks of a large number of serious illnesses to save test subjects at a stage of ahead-disease, by associating relationships between various gastrointestinal diseases and the time-dependent change characteristics of defecation gas data in the data analyzer 12b on a server side (FIG. 15).

According to the biological information system of the present embodiment, similarity is determined on the basis of the time-dependent change characteristics of correlations of odiferous gas and healthy-state gas (FIG. 19), so that an influence of noise or the like included at the time of measurement is hardly exerted, and an unnecessary mental burden can be prevented from being applied to a test subject by notifying the test subject of an erroneous analysis result.

According to the biological information measurement system of the present embodiment, similarity is determined at a time point when the latest test subject defecation gas data of a test subject is deteriorated to a predetermined physical condition stage (FIGS. 18 and 21), so that when the risk is increased to such an extent that an affection risk should be notified, proper notification can be given to the test subject.

According to the biological information measurement system of the present embodiment, the notification mode and the person to be notified are changed (FIG. 21) in accordance with the physical condition stage to which the latest test subject defecation gas data of a test subject belongs, so that a proper person can be notified of proper information at a proper timing.

According to the biological information measurement system of the present embodiment, the analysis result of physical condition is notified in more detail (FIG. 22) as the physical condition stage of a test subject is closer to a state with concern for a disease than in a case where the physical condition of the test subject is relatively good (FIG. 7, FIG. 8), so that the test subject can recognize the state of health of himself or herself in detail when the test subject has physical condition that requires immediate medical treatment, and the test subject can be given a strong motivation for undergoing diagnosis in a medical facility or the like.

According to the biological information measurement system of the present embodiment, the notification means notifies a disease which might affect a test subject, and a risk of the test subject being affected after a predetermined period (FIG. 22), when the physical condition stage to which the latest test subject defecation gas data of the test subject is on a side with more concern for the disease than a predetermined physical condition stage, and notifies whether or not physical condition is improved when latest physical condition stage is on a side of a healthy state (FIG. 7, FIG. 8). Consequently, proper information is notified in accordance with the physical condition stage of the test subject, so that the test subject performs proper health care, and can undergo diagnosis and medical treatment in accordance with necessity.

According to the biological information measurement system of the present embodiment, the reference affected test subject defecation gas data (FIG. 16) to be a reference concerning a disease is generated on the basis of the test subject defecation gas data of a plurality of test subjects affected by the same disease, so that an accurate reference can be set for a predetermined disease, and an affection risk can be estimated properly on the basis of similarity to the test subject defecation gas data.

According to the biological information measurement system of the present embodiment, the accuracy of the affected test subject defecation gas data is notified in addition (FIG. 23), so that the test subject, a doctor and the like can recognize accuracy of the notified affection risk more objectively, and an unnecessary mental burden can be prevented from being applied to the test subject by a reference with low accuracy.

According to the biological information measurement system of the present embodiment, as the health care information, the state of health of the test subject is displayed in the physical condition table (FIG. 8) equipped with the first index and the second index, so that the test subject can understand the state of health of himself or herself from many aspects.

According to the biological information measurement system of the present embodiment, notification is performed so that the test subject can determine the timing at which the risk of being affected becomes high (upper section in FIG. 22), and the risk reduction information is notified (lower section in FIG. 22). Consequently, the test subject can recognize the affection risk of himself or herself more specifically, so that a clear motivation for undergoing diagnosis in a medical facility and undergoing medical treatment can be given to the test subject, and the test subject can immediately recognize the measure for recovering physical condition of himself or herself, so that the test subject can make an effort to recover physical condition at an early stage.

According to the biological information measurement system of the present embodiment, the odiferous gas attached to a test subject detected before a defecation act is started is also detected, and the data (FIG. 24) on the odiferous gas is also used in analysis of the physical condition of the test subject, so that it becomes possible to use the biological information measurement system in evaluation of affection risks of a larger number of diseases.

According to the biological information measurement system of the present embodiment, the data analyzer 12b on a server side analyzes the situation of prevalence of the disease on the basis of the information on diarrhea collected from the respective devices 10 on a test subject side (FIG. 25), and if it is determined that the disease is prevalent, the server side data analyzer allows the notification means to notify that the diseases is prevalent (FIG. 26), so that it becomes possible to notify facilities concerned of the occurrence of mass food poisoning at an early stage to cope with the mass food poisoning quickly.

According to the biological information measurement system of the present embodiment, a specific person or a facility set in advance is notified of the information on a specific test subject set in advance, or a test subject living in a specific district (FIG. 27), so that it becomes possible to monitor the state of health of a test subject who is suspected to be infected with an infectious disease or the like, and it becomes possible to take measures to stop spread of the disease at an early stage.

What is claimed is:

1. A biological information measurement system that measures physical condition of a test subject on the basis of defecation gas discharged into a bowl of a flush toilet, the biological information measurement system comprising a test subject side device provided in a room where the flush toilet is installed, and a server communicable with the test subject side device,
wherein the test subject side device comprises
a suction device that sucks gas in the bowl into which the defecation gas is discharged during a defecation act of the test subject,
a gas detector that is sensitive to methyl mercaptan gas that is odiferous gas containing a sulfur component and odiferous gas other than methyl mercaptan gas, which are contained in the gas sucked by the suction device, and outputs first detection data,
a test subject identification device that accepts input of test subject identification information,
a control device that controls the suction device and the gas detector, and
a communication device that transmits the first detection data of the odiferous gas detected by the gas detector to the server,
the server comprises
a database in which test subject defecation gas data including the first detection data of the odiferous gas transmitted from a plurality of the test subject side devices, the test subject identification information and dates and times of the defecation acts, and test subject disease data concerning diseases which affect a plurality of test subjects using the test subject side devices, which is acquired from a medical facility, are accumulated and recorded, and
a server side data analyzer that analyzes the physical condition of the test subject on the basis of the test subject defecation gas data and the test subject disease data which are accumulated and recorded in the database, and
the server side data analyzer comprises
relating means that generates affected test subject defecation gas data by relating the test subject disease data of the test subject affected by a predetermined disease, and the test subject defecation gas data of the test subject with each other, and accumulates the affected test subject defecation gas data in the database,
similarity determination means that compares a time-dependent change characteristic of the affected test subject defecation gas data accumulated in the database, and a time-dependent change characteristic of the test subject defecation gas data of a specific test subject transmitted from the test subject side device, and determines whether or not the time-dependent change characteristic of the affected test subject defecation gas data and the time-dependent change characteristic of the test subject defecation gas data are similar to each other, and
notification means that performs a predetermined notification to the test subject or a previously registered person who relates to said test subject, associated with the test subject defecation gas data which is determined as having similarity to the affected test subject defecation gas data by the similarity determination means.

2. The biological information measurement system according to claim 1, wherein the gas detector is configured to detect healthy-state gas composed of at least one of hydrogen gas, carbon dioxide gas, methane gas and acetic acid gas, contained in the defecation gas sucked by the suction device to output second detection data, the communication device is configured to transmit the second detection data of the healthy-state gas to the server with the first detection data, and the similarity determination means determines whether or not the affected test subject defecation gas data and the test subject defecation gas data are similar to each other on the basis of a time-dependent change characteristic of a correlation of the odiferous gas and the healthy-state gas.

3. The biological information measurement system according to claim 2,
wherein for an analysis result of physical condition of the test subject by the server side data analyzer, a plurality of physical condition stages, from a healthy state to a state with concern for disease, are set, and the similarity determination means determines similarity at a time point at which a latest test subject defecation gas data of a specific test subject is deteriorated to a predetermined physical condition stage.

4. The biological information measurement system according to claim 3,
wherein a notification mode of the notification by the notification means is varied in accordance with the physical condition stage based on the latest test subject defecation gas data of the test subject.

5. The biological information measurement system according to claim 4,
wherein the notification means changes a person to be notified, in accordance with the physical condition stage based on the latest test subject defecation gas data of the test subject.

6. The biological information measurement system according to claim 5,
wherein the notification means notifies the analysis result of physical condition in more detail, as the physical condition stage based on the latest test subject defecation gas data of the test subject is closer to the state with concern for a disease.

7. The biological information measurement system according to claim 6,
wherein the notification means notifies a disease which might affect the test subject, and a risk of the test subject being affected after a predetermined period, when the physical condition stage based on the latest test subject defecation gas data of the test subject is on a side with more concern for the disease than a predetermined physical condition stage, and notifies whether or not physical condition is improved when the latest physical condition stage is on a side of the healthy state.

8. The biological information measurement system according to claim 7,
wherein the relating means generates reference affected test subject defecation gas data to be a reference concerning a disease on the basis of the test subject defecation gas data of a plurality of test subjects affected by the same disease, and the similarity determination means compares time-dependent change characteristics of the reference affected test subject defecation gas data and the test subject defecation gas data, and determines whether or not the time-dependent change characteristics are similar to each other.

9. The biological information measurement system according to claim 8,
wherein the relating means is configured to classify test subjects using the test subject side devices into a plurality of groups, and generate the reference affected test subject defecation gas data for each of the groups, and the similarity determination means compares the test subject defecation gas data with the reference affected test subject defecation gas data generated in the group to which the test subject belongs, and determines whether or not the test subject defecation gas data is similar to the reference affected test subject defecation gas data.

10. The biological information measurement system according to claim 9,
wherein the groups are classified on the basis of at least one of age, sex, district, occupation and a living environment.

11. The biological information measurement system according to claim 4,
wherein the relating means is configured to evaluate accuracy of the affected test subject defecation gas data generated, and the notification means notifies the accuracy in addition.

12. The biological information measurement system according to claim 11,
wherein the notification means changes a timing for performing notification, or a content of the notification, in accordance with accuracy of the affected test subject defecation gas data to which the similarity is determined by the similarity determination means.

13. The biological information measurement system according to claim 4,
wherein the server side data analyzer is configured to allow the notification means to notify at least two kinds of information of health care information visualizing a change in the test subject defecation gas data in a time-dependent manner, and information based on determination of similarity to the affected test subject defecation gas data, so that a change in a state of health of the test subject can be recognized.

14. The biological information measurement system according to claim 13,
wherein the health care information is displayed so that the change in the state of health of the test subject can be recognized in a time-dependent manner, as a point in a physical condition display table provided with a first index based on the first detection data, and a second index based on the second detection data, and the server is configured to update the physical condition display table on the basis of inputted information on the test subject.

15. The biological information measurement system according to claim 4,
wherein the notification means is configured to perform notification so that the test subject can determine a timing at which a risk of the test subject being affected becomes high, with similarity of the test subject defecation gas data of the test subject and the affected test subject defecation gas data, which is determined by the similarity determination means.

16. The biological information measurement system according to claim 15,
wherein the server side data analyzer further allows risk reduction information that is an attention for reducing a risk of the test subject being affected to be presented by the notification means.

17. The biological information measurement system according to claim 16,
wherein in the database, the test subject defecation gas data of respective test subjects, and the risk reduction information executed by the test subjects are recorded by being related with each other and accumulated, and the server side data analyzer allows the risk reduction information having a large risk reduction effect to be provided preferentially by the notification means.

18. The biological information measurement system according to claim 4,
wherein the gas detector is configured to also detect odiferous gas attached to the test subject which is detected before the test subject starts the defecation act, the communication device transmits detection data concerning the odiferous gas attached to the test subject to the server with the first detection data, and the server side data analyzer also uses the detection data concerning the odiferous gas attached to the test subject in analysis of physical condition of the test subject.

19. The biological information measurement system according to claim 4,
wherein the test subject side device further comprises diarrhea detection means capable of detecting diarrhea of the test subject, the communication device is configured to transmit information on diarrhea of the test subject to the server, the server side data analyzer analyzes a situation of prevalence of a disease on the basis of information on diarrhea collected from the respective test subject side devices, and when it is determined that the disease is prevalent, the server side data analyzer allows the notification means to notify that the disease is prevalent.

20. The biological information measurement system according to claim 4,
wherein the server side data analyzer notifies a specific person or a facility as previously registered, of information on a specific test subject as previously registered, or test subjects living in a specific district, which is acquired by the test subject side device, and is transmitted from the communication devices.

21. A server for biological information measurement that measures physical condition of a test subject on the basis of defecation gas discharged into a bowl of a flush toilet, comprising:
a receiver that receives first detection data concerning methyl mercaptan gas which is odiferous gas containing a sulfur component in the defecation gas and odiferous gas other than methyl mercaptan gas, measured in a test subject side device;
a database in which test subject defecation gas data including the first detection data of the odiferous gas, transmitted from a plurality of test subject side devices, test subject identification information for identifying test subjects, and dates and times when the first detection data are acquired, and test subject disease data concerning diseases which affect a plurality of test subjects who use the test subject side devices, acquired from a medical facility are accumulated and recorded; and
a server side data analyzer that analyzes physical condition of the test subject on the basis of the test subject defecation gas data and the test subject disease data accumulated and recorded in the database,
wherein the server side data analyzer comprises
relating means that generates affected test subject defecation gas data by relating the test subject disease data of the test subject affected by a predetermined disease, and the test subject defecation gas data of the test subject with each other, and accumulates the affected test subject defecation gas data in the database,
similarity determination means that compares a time-dependent change characteristic of the affected test subject defecation gas data accumulated in the database, and a time-dependent change characteristic of the test subject defecation gas data of the specific test subject transmitted from the test subject side device, and determines whether or not the time-dependent change characteristics of the affected test subject defecation gas data and the test subject defecation gas data of the specific test subject are similar to each other, and
notification means that performs a predetermined notification to the test subject or a previously registered person who relates to said test subject, associated with the test subject defecation gas data which is determined as having similarity to the affected test subject defecation gas data by the similarity determination means.
